(19) United States
(12) Patent Application Publication
(10) Pub. No.: US 2022/0223798 A1
(43) Pub. Date: Jul. 14, 2022

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Takushi SHIOMI, Sodegaura-shi (JP); Hisato MATSUMOTO, Sodegaura-shi (JP); Yukitoshi JINDE, Sodegaura-shi (JP); Kengo KISHINO, Sodegaura-shi (JP); Kazuki TERADA, Sodegaura-shi (JP); Keiichi YASUKAWA, Sodegaura-shi (JP); Toshinari OGIWARA, Sodegaura-shi (JP)

(73) Assignee: IDEMITSU KOSAN CO.,LTD., Tokyo (JP)

(21) Appl. No.: 17/644,636

(22) Filed: Dec. 16, 2021

(30) Foreign Application Priority Data
Dec. 17, 2020 (JP) 2020-209668
Jan. 18, 2021 (JP) 2021-005799

(51) Int. Cl. H01L 51/00; C07D 495/04; C09K 11/06; C07F 5/02

(52) U.S. Cl. CPC H01L 51/0071; C07D 495/04; H01L 51/5012; C07F 5/027; H01L 51/008; C09K 11/06

(57) ABSTRACT

An organic electroluminescence device includes an anode, a cathode, and an emitting layer provided between the anode and the cathode, in which the emitting layer contains a delayed fluorescent compound M2 represented by a formula (2) and a compound M3 represented by a formula (3), and a singlet energy Si(M2) of the compound M2 and a singlet energy Si(M3) of the compound M3 satisfy a relationship of a numerical formula (Numerical Formula 1). In the formula (3), Y₃₁ to Y₃₆ are each independently CR₃ or a nitrogen atom, and two or more of Y₃₁ to Y₃₆ are each a nitrogen atom,

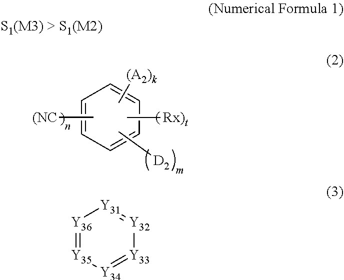

The entire disclosure of Japanese Patent Applications No. 2020-209668 filed Dec. 17, 2020, and No. 2021-005799 filed Jan. 18, 2021 are expressly incorporated by reference herein.

### TECHNICAL FIELD

The present invention relates to an organic electroluminescence device and an electronic device.

### BACKGROUND ART

When a voltage is applied to an organic electroluminescence device (hereinafter, occasionally referred to as “organic EL device”), holes are injected from an anode and electrons are injected from a cathode into an emitting layer. The injected electrons and holes are recombined in the emitting layer to form excitons.

Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Accordingly, studies has been made to improve a performance of the organic EL device. The performance of the organic EL device is evaluable in terms of, for instance, luminance, emission wavelength, chromaticity, luminous efficiency, drive voltage, and lifetime.

For instance, it is expected to further efficiently emit the organic EL device using triplet excitons in addition to singlet excitons. In view of the above, a highly efficient fluorescent organic EL device using thermally activated delayed fluorescence (hereinafter, sometimes simply referred to as “delayed fluorescence”) has been proposed and studied.

A TADF (Thermally Activated Delayed Fluorescence) mechanism uses such a phenomenon that inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI, Chihaya, published by Kodansha, issued on Apr. 1, 2012, on pages 261-268).

As a compound exhibiting thermally activated delayed fluorescence (TADF properties) (hereinafter sometimes referred to as a delayed fluorescent compound or TADF compound), for example, a compound in which a donor moiety and an acceptor moiety are bonded in a molecule is known.

For instance, Literature 1 (International Publication No. WO2019/195104), Literature 2 (International Publication No. WO 2018/155642), and Literature 3 (International Publication No. WO 2019/107934) disclose an organic electroluminescence device using a delayed fluorescent compound.

In order to improve performance of an electronic device such as a display, an organic electroluminescence device has been required to be further improved in performance.

### SUMMARY OF THE INVENTION

An object of the invention is to provide an organic electroluminescence device having a high performance, especially high luminous efficiency, and an electronic device including the organic electroluminescence device.

According to an aspect of the invention, there is provided an organic electroluminescence device including an anode, a cathode, and an emitting layer provided between the anode and the cathode, in which the emitting layer includes a delayed fluorescent compound M2 represented by a formula (2) below and a compound M3 represented by a formula (3) below, and a singlet energy S₁(M2) of the compound M2 and a singlet energy S₁(M3) of the compound M3 satisfy a relationship of a numerical formula (Numerical Formula 1) below.

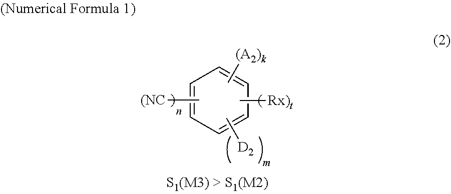

In the formula (2):

k is 1, 2, 3, or 4;

m is 1, 2, 3, or 4;

n is 1 or 2;

t is 0, 1, 2, or 3;

k+m+n+t is 6;

when t is 2 or 3, a plurality of Rx are mutually the same or different;

A₂ is a group represented by a formula (21) below;

when k is 2, 3, or 4, a plurality of A₂ are mutually the same or different;

D₂ is a group represented by a formula (22) below;

when m is 2, 3, or 4, a plurality of D₂ are mutually the same or different; and

CN is a cyano group.

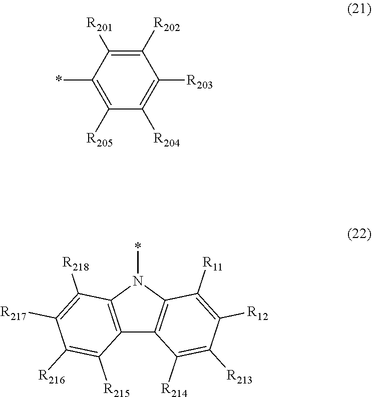

In the formula (21): at least one combination of adjacent two or more of R₂₀₁ to R₂₀₅ are mutually bonded to form a substituted or unsubstituted monocyclic ring, mutually bonded to form a substituted or unsubstituted fused ring, or not mutually bonded.

In the formula (22): at least one combination of adjacent two or more of R₂₁₁ to R₂₁₈ are mutually bonded to form a substituted or unsubstituted monocyclic ring, mutually bonded to form a substituted or unsubstituted fused ring, or not mutually bonded.

* in each of the formulae (21) and (22) represents a bonding position to a benzene ring in the formula (2).

Rx in the formula (2), R₂₀₁ to R₂₀₅ not forming the substituted or unsubstituted monocyclic ring and not forming the substituted or unsubstituted fused ring in the formula (21), and R₂₁₁ to R₂₁₈ not forming the substituted or unsubstituted monocyclic ring and not forming the substituted or unsubstituted fused ring in the formula (22) are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms.

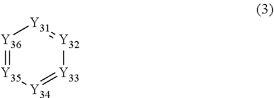

In the formula (3):

Y₃₁ to Y₃₆ are each independently CR₃ or a nitrogen atom;

two or more of Y₃₁ to Y₃₆ are each a nitrogen atom;

when a plurality of R₃ are present, at least one combination of adjacent two or more of the plurality of R₃ are mutually bonded to form a substituted or unsubstituted monocyclic ring, mutually bonded to form a substituted or unsubstituted fused ring, or not mutually bonded; and

R₃ not forming the substituted or unsubstituted monocyclic ring and not forming the substituted or unsubstituted fused ring is each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, a substituted or unsubstituted heterocyclic group having 5 to 50 ring atoms, or a group represented by a formula (3A) below.

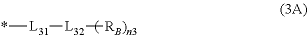

In the formula (3A):

R_{B} is a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms;

when a plurality of R_{B} are present, the plurality of R_{B} are mutually the same or different;

L₃₁ is: a single bond;
a substituted or unsubstituted arylene group having 6 to 50 ring carbon atoms, or a trivalent, tetravalent, pentavalent, or hexavalent group derived from the arylene group;
a substituted or unsubstituted divalent heterocyclic group having 5 to 50 ring atoms, or a trivalent, tetravalent, pentavalent, or hexavalent group derived from the heterocyclic group; or
a divalent group formed by bonding two groups selected from the group consisting of a substituted or unsubstituted arylene group having 6 to 50 ring carbon atoms and a substituted or unsubstituted divalent heterocyclic group having 5 to 50 ring atoms, or a trivalent, tetravalent, pentavalent, or hexavalent group derived from the divalent group;

L₃₂ is a single bond, a substituted or unsubstituted arylene group having 6 to 50 ring carbon atoms, or a substituted or unsubstituted divalent heterocyclic group having 5 to 50 ring atoms;

n₃ is 1, 2, 3, 4, or 5;

when L₃₁ is a single bond, n₃ is 1, and L₃₂ is bonded to a carbon atom in a six-membered ring in the formula (3);

when a plurality of L₃₂ are present, the plurality of L₃₂ are mutually the same or different; and
represents a bonding position to a carbon atom in a six-membered ring in the formula (3).

When the compound M3 is a compound Mx3 represented by a formula (301) below and a singlet energy S₁(Mx3) of the compound Mx3 is larger than a singlet energy S₁(M2) of the compound M2, the emitting layer does not contain a compound having a larger singlet energy than the singlet energy S₁(M2) of the compound M2, other than the compound Mx3.

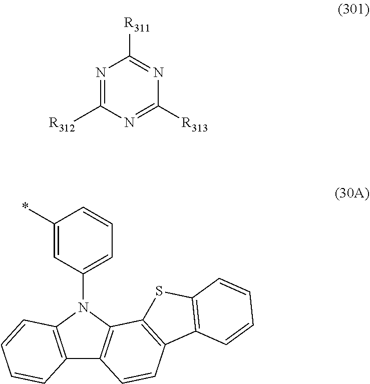

In the formula (301): R₃₁₁ is a phenyl structure; R₃₁₂ is a biphenyl structure; and R₃₁₃ is a group represented by the formula (30A) below.

In the compound M2 and the compound M3, R₉₀₁, R₉₀₂, R₉₀₃, R₉₀₄, R₉₀₅, R₉₀₆, R₉₀₇, R₉₀₈, R₉₀₉, R₉₃₁, R₉₃₂, R₉₃₃, R₉₃₄, R₉₃₅, R₉₃₆, and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₉₀₈ are present, the plurality of R₉₀₈ are mutually the same or different;

when a plurality of R₉₀₉ are present, the plurality of R₉₀₉ are mutually the same or different;

when a plurality of R₉₃₁ are present, the plurality of R₉₃₁ are mutually the same or different;

when a plurality of R₉₃₂ are present, the plurality of R₉₃₂ are mutually the same or different;

when a plurality of R₉₃₃ are present, the plurality of R₉₃₃ are mutually the same or different;

when a plurality of R₉₃₄ are present, the plurality of R₉₃₄ are mutually the same or different;

when a plurality of R₉₃₅ are present, the plurality of R₉₃₅ are mutually the same or different;

when a plurality of R₉₃₆ are present, the plurality of R₉₃₆ are mutually the same or different; and

when a plurality of R₉₃₇ are present, the plurality of R₉₃₇ are mutually the same or different.

According to another aspect of the invention, an electronic device including the organic electroluminescence device according to the above aspect of the invention is provided.

According to the above aspects of the invention, an organic electroluminescence device having a high performance, especially high luminous efficiency, and an electronic device including the organic electroluminescence device can be provided.

### BRIEF DESCRIPTION OF DRAWING(S)

FIG. 1 schematically shows an exemplary arrangement of an organic electroluminescence device according to a first exemplary embodiment of the invention.

FIG. 2 schematically shows a device that measures transient PL.

FIG. 3 shows an example of a decay curve of the transient PL.

FIG. 4 shows a relationship of an energy level between a compound M3 and a compound M2 in an emitting layer in an exemplary arrangement of the organic electroluminescence device according to the first exemplary embodiment of the invention.

FIG. 5 shows a relationship of an energy level and energy transfer between a compound M3, a compound M2, and a compound M1 in an emitting layer in an exemplary arrangement of an organic electroluminescence device according to a second exemplary embodiment of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a protium.

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded with each other to form the ring. When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine group has 5 ring carbon atoms, and a furan group has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent are not counted as the pyridine ring atoms. Accordingly, a pyridine ring bonded with a hydrogen atom(s) or a substituent(s) has 6 ring atoms. For instance, the hydrogen atom(s) bonded to a quinazoline ring or the atoms forming a substituent are not counted as the quinazoline ring atoms. Accordingly, a quinazoline ring bonded with hydrogen atom(s) or a substituent(s) has ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). (Herein, an unsubstituted aryl group refers to an “unsubstituted aryl group” in a “substituted or unsubstituted aryl group,” and a substituted aryl group refers to a “substituted aryl group” in a “substituted or unsubstituted aryl group.” A simply termed “aryl group” herein includes both of an “unsubstituted aryl group” and a “substituted aryl group.”

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by substituting a hydrogen atom bonded to a carbon atom of a skeleton of a “substituted aryl group” in the specific example group G1B below, and a group derived by substituting a hydrogen atom of a substituent of the “substituted aryl group” in the specific example group G1B below.

### Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, a perylenyl group, and a monovalent aryl group derived by removing one hydrogen atom from cyclic structures represented by formulae (TEMP-1) to (TEMP-15) below.

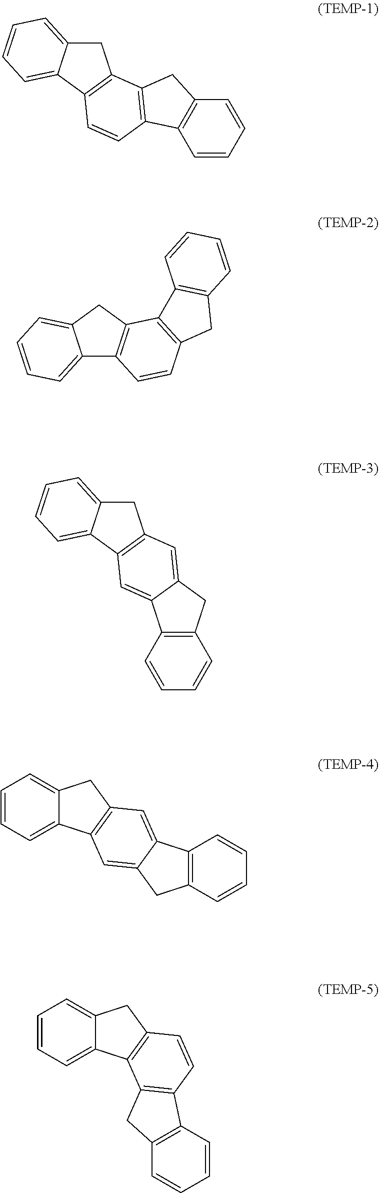

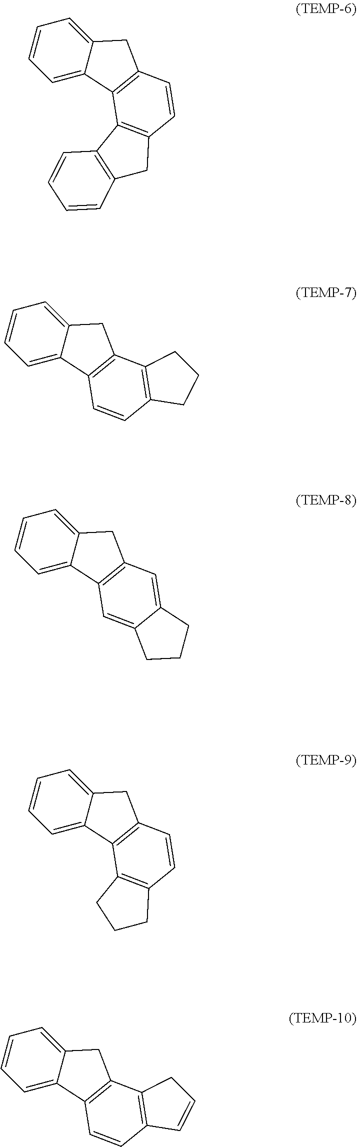

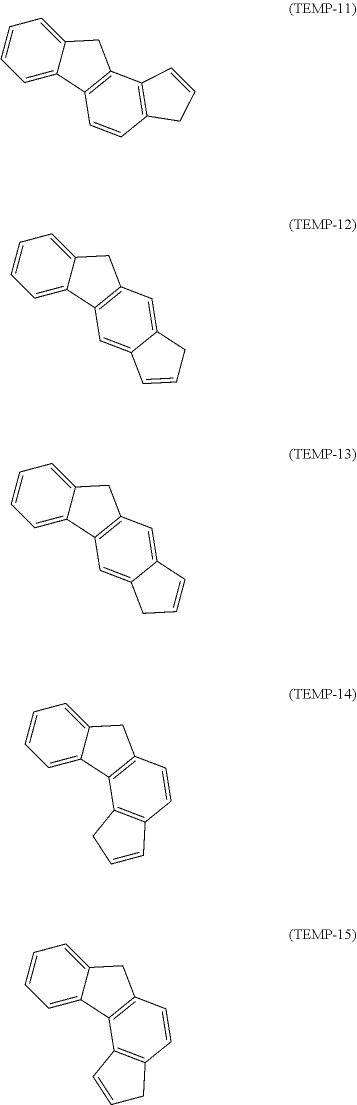

### Substituted Aryl Group (Specific Example Group G1B):

o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and a group derived by substituting at least one hydrogen atom of a monovalent group derived from the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of “unsubstituted heterocyclic group” and “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by substituting a hydrogen atom bonded to a ring atom of a skeleton of a “substituted heterocyclic group” in the specific example group G2B below, and a group derived by substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, a pyridyl group, pyridazynyl group, <<nret>. a pyrimidinyl group, pyrazinyl group, a triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

furyl group, oxazolyl group, isoxazolyl group, oxadiazolyl group, xanthenyl group, benzofuranyl group, isobenzofuranyl group, a dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

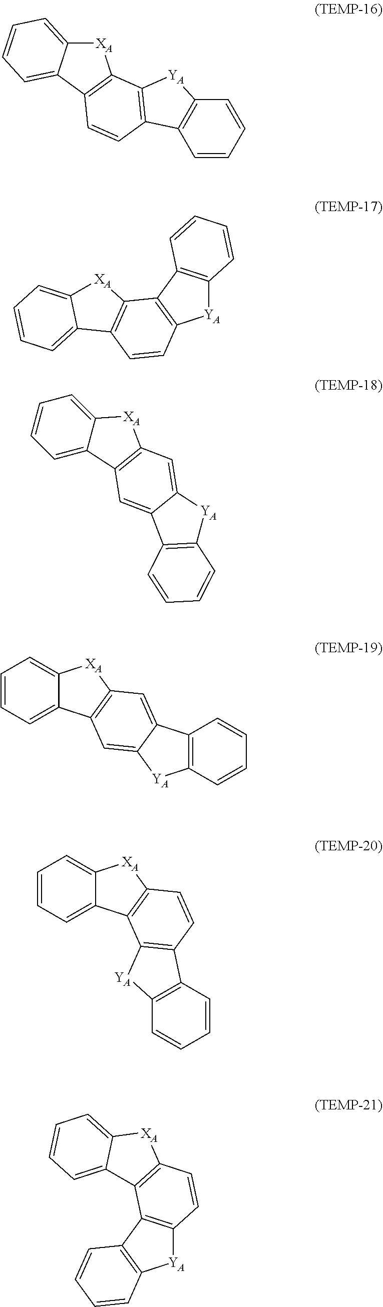

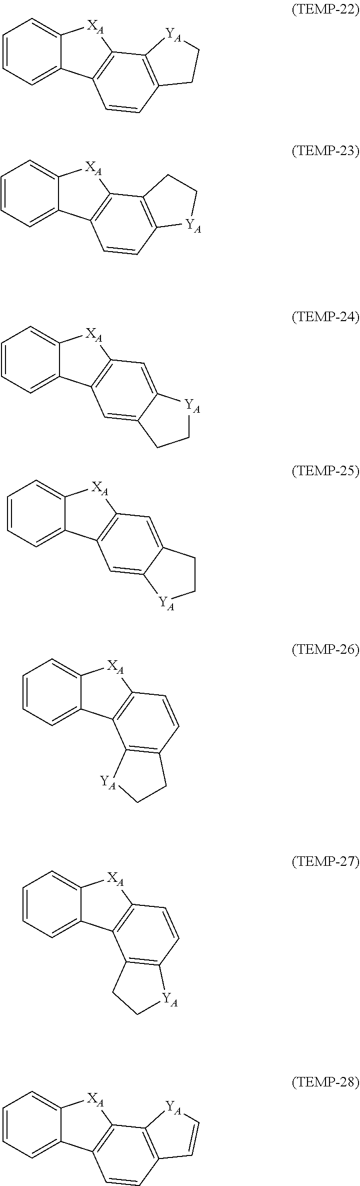

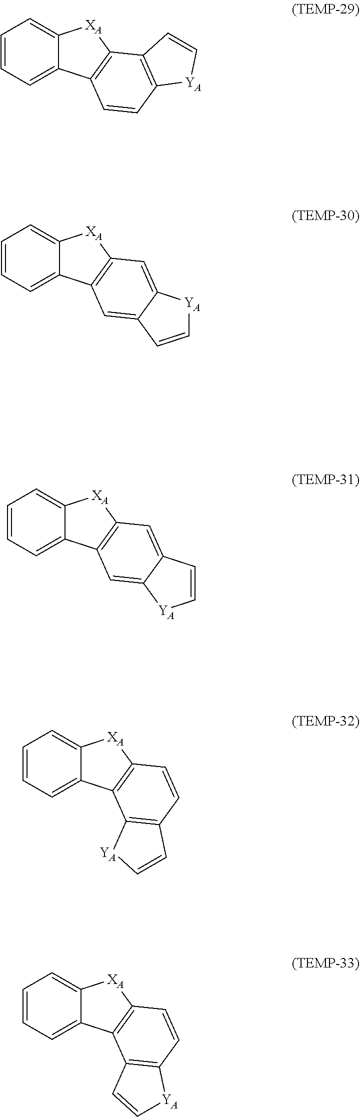

In the formulae (TEMP-16) to (TEMP-33), X_{A} and Y_{A} are each independently an oxygen atom, a sulfur atom, NH, or CH₂. However, at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH, or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

phenyldibenzothiophenyl group, methyldibenzothiophenyl group, t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B below). (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of “unsubstituted alkyl group” and “substituted alkyl group.”

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by substituting a hydrogen atom bonded to a carbon atom of a skeleton of the “substituted alkyl group” in the specific example group G3B, and a group derived by substituting a hydrogen atom of a substituent of the “substituted alkyl group” in the specific example group G3B.

### Unsubstituted Alkyl Group (Specific Example Group G3A):

methyl group, ethyl group, n-propyl group, isopropyl group, n-butyl group, isobutyl group, s-butyl group, and t-butyl group.

### Substituted Alkyl Group (Specific Example Group G3B):

heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of “unsubstituted alkenyl group” and “substituted alkenyl group.”

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by substituting a hydrogen atom of a skeleton of the “substituted alkenyl group” in the specific example group G4B with a substituent, and a group derived by substituting a hydrogen atom of a substituent of the “substituted alkenyl group” in the specific example group G4B with a substituent.

### Unsubstituted Alkenyl Group (Specific Example Group G4A):

vinyl group, allyl group, 1-butenyl group, 2-butenyl group, and 3-butenyl group.

### Substituted Alkenyl Group (Specific Example Group G4B):

1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group,” and a substituted alkynyl group refers to a “substituted alkynyl group” in a “substituted or unsubstituted alkynyl group.”) A simply termed “alkynyl group” herein includes both of “unsubstituted alkynyl group” and “substituted alkynyl group.”

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A):

an ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group.”

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

4-methylcyclohexyl group.

Group Represented by —Si(R₉₀₁)(R₉₀₂)(R₉₀₃)

Specific examples (specific example group G7) of a group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6).

Herein: G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

A plurality of G1 in —Si(G1)(G1)(G1) are mutually the same or different.

A plurality of G2 in —Si(G1)(G2)(G2) are mutually the same or different.

A plurality of G1 in —Si(G1)(G1)(G2) are mutually the same or different.

A plurality of G2 in —Si(G2)(G2)(G2) are mutually the same or different.

The plurality of G3 in —Si(G3)(G3)(G3) are mutually the same or different.

A plurality of G6 in —Si(G6)(G6)(G6) are mutually the same or different.

### Group Represented by —O—(R₉₀₄)

Specific examples (specific example group G8) of a group represented by —O—(R₉₀₄) herein include —O(G1); —O(G2); —O(G3); and —O(G6).

Herein: G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include; —S(G1); —S(G2); —S(G3); and —S(G6).

Herein: G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6).

Herein: G1 represents a “substituted or unsubstituted aryl group” in the specific example group G1;

G2 represents a “substituted or unsubstituted heterocyclic group” in the specific example group G2;

G3 represents a “substituted or unsubstituted alkyl group” in the specific example group G3; and

G6 represents a “substituted or unsubstituted cycloalkyl group” in the specific example group G6.

A plurality of G1 in —N(G1)(G1) are mutually the same or different.

A plurality of G2 in —N(G2)(G2) are mutually the same or different.

A plurality of G3 in —N(G3)(G3) are mutually the same or different.

A plurality of G6 in —N(G6)(G6)) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein includes a group derived by substituting at least one hydrogen atom bonded to a carbon atom of an alkyl chain of a “substituted fluoroalkyl group” with a substituent, and a group derived by substituting at least one hydrogen atom of a substituent of the “substituted fluoroalkyl group” with a substituent. Specific examples of the “substituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein includes a group derived by substituting at least one hydrogen atom bonded to a carbon atom of an alkyl chain of a “substituted haloalkyl group” with a substituent, and a group derived by substituting at least one hydrogen atom of a substituent of the “substituted haloalkyl group” with a substituent. Specific examples of the “substituted haloalkyl group” include a group derived by substituting at least one hydrogen atom of the “alkyl group” (specific example group G3) with a halogen atom. The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. The plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by (G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

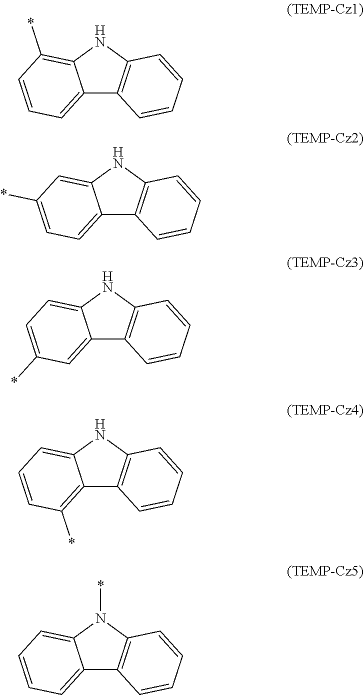 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

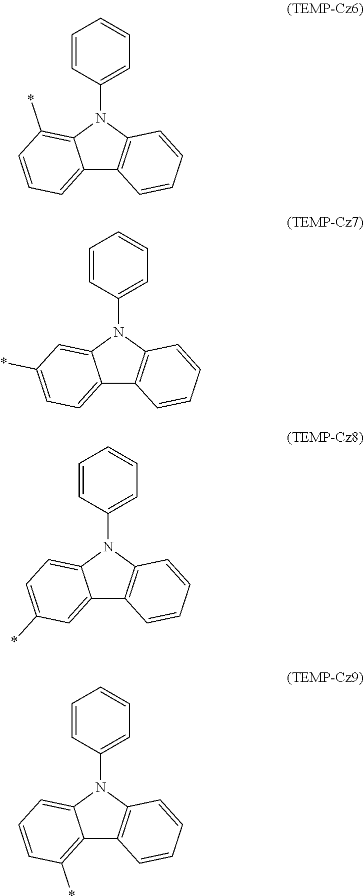

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

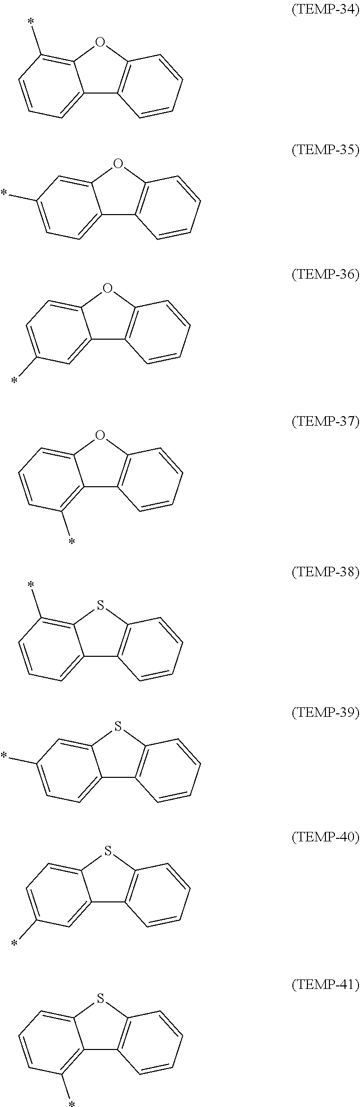

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocyclic ring of the “substituted or unsubstituted heterocyclic group.” Specific examples of the “substituted or unsubstituted heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl ring of the “substituted or unsubstituted alkyl group.” Specific examples of the “substituted or unsubstituted alkylene group” (specific example group G14) include a divalent group derived by removing one hydrogen atom on an alkyl ring of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

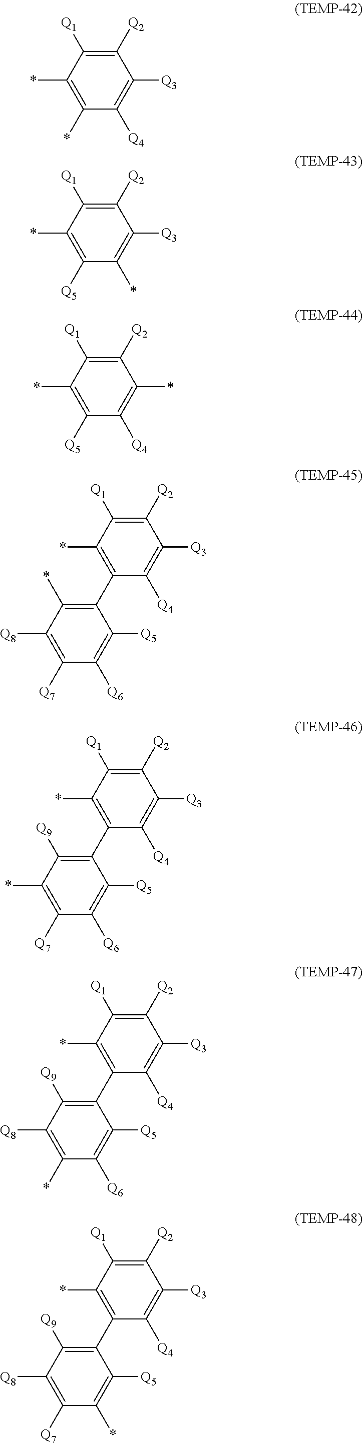

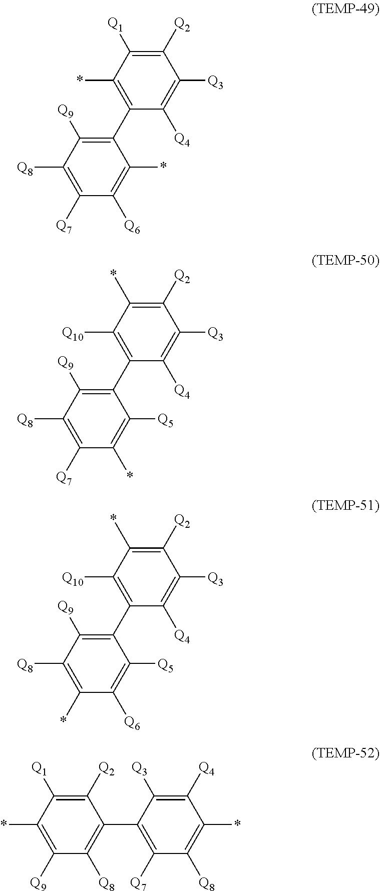

In the formulae (TEMP-42) to (TEMP-52), Q₁ to Q₁₀ each independently are a hydrogen atom or a substituent.

In the formulae (TEMP-42) to (TEMP-52), * represents a bonding position.

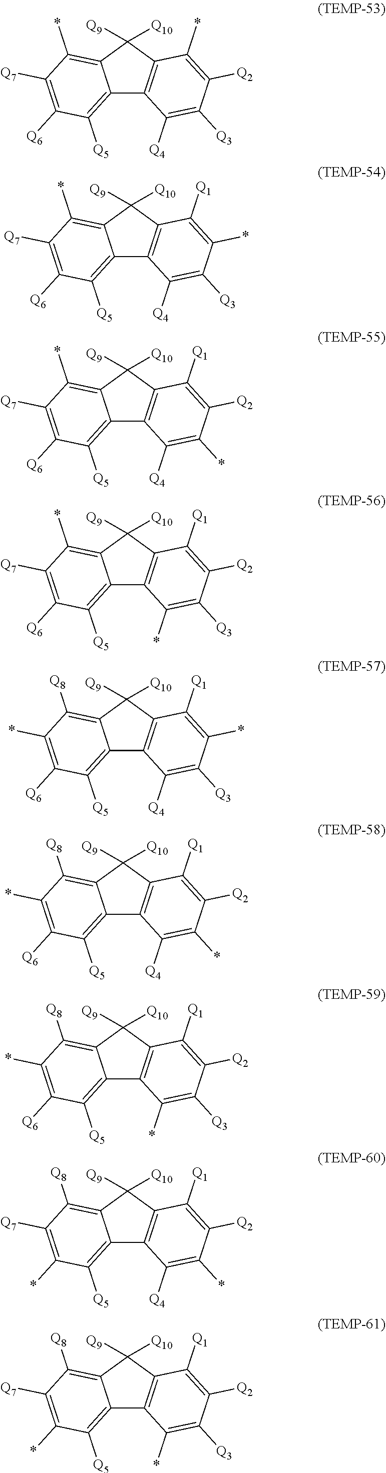

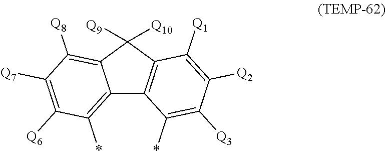

In the formulae (TEMP-53) to (TEMP-62), Q₁ to Q₁₀ each independently are a hydrogen atom or a substituent.

In the formulae, Q₉ and Q₁₀ may be mutually bonded through a single bond to form a ring.

In the formulae (TEMP-53) to (TEMP-62), * represents a bonding position.

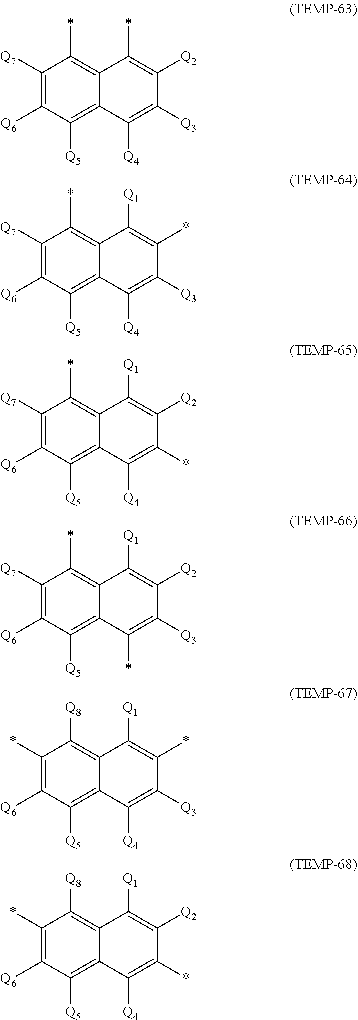

In the formulae (TEMP-63) to (TEMP-68), Q₁ to Q₈ each independently are a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

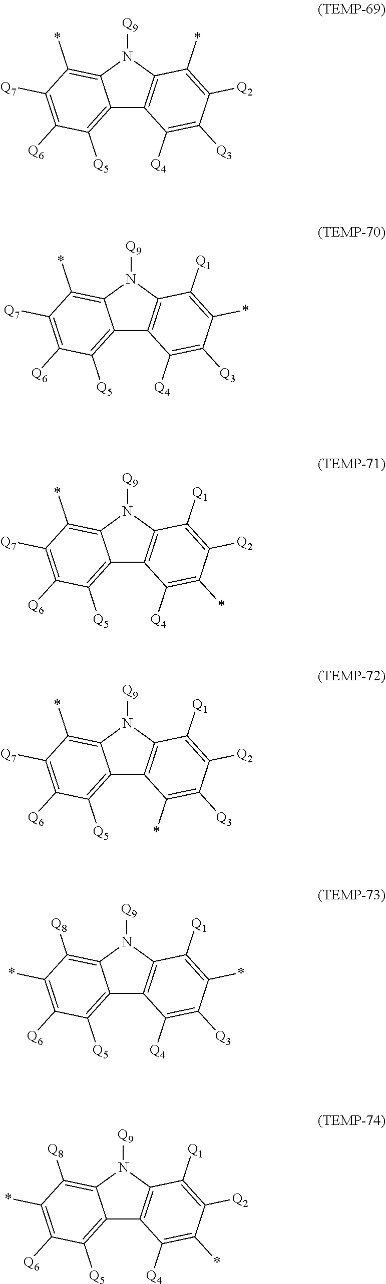

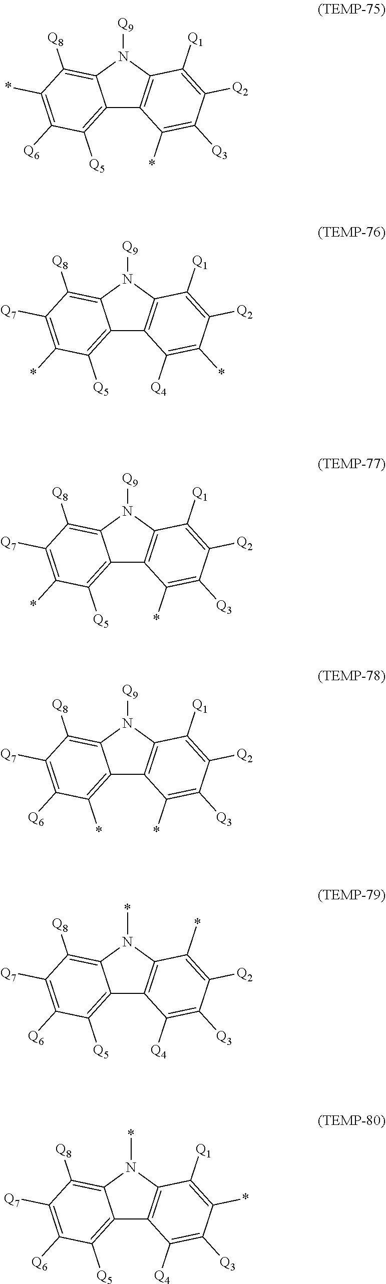

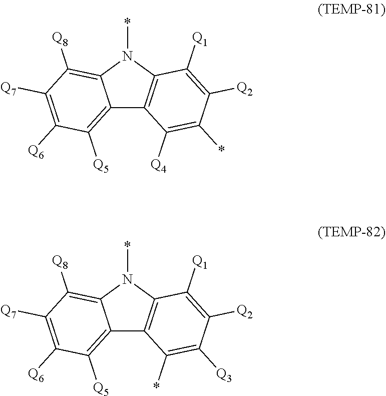

In the formulae (TEMP-69) to (TEMP-82), Q₁ to Q₉ are each independently a hydrogen atom or a substituent.

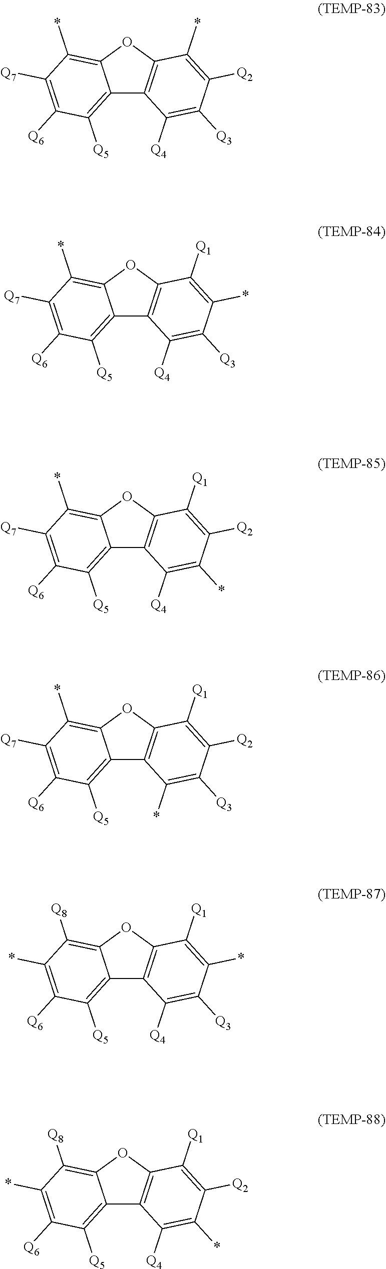

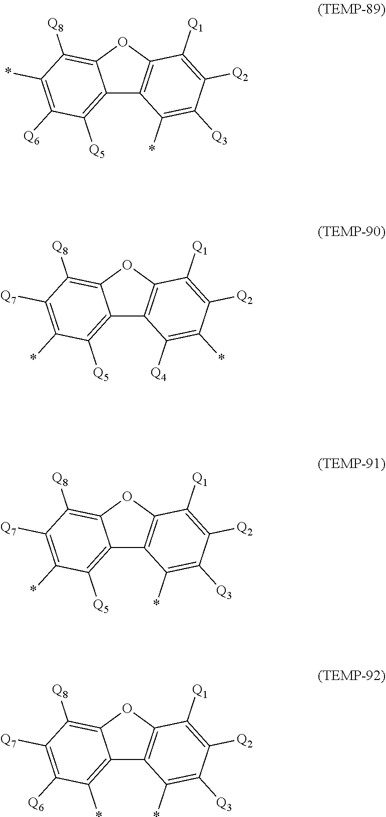

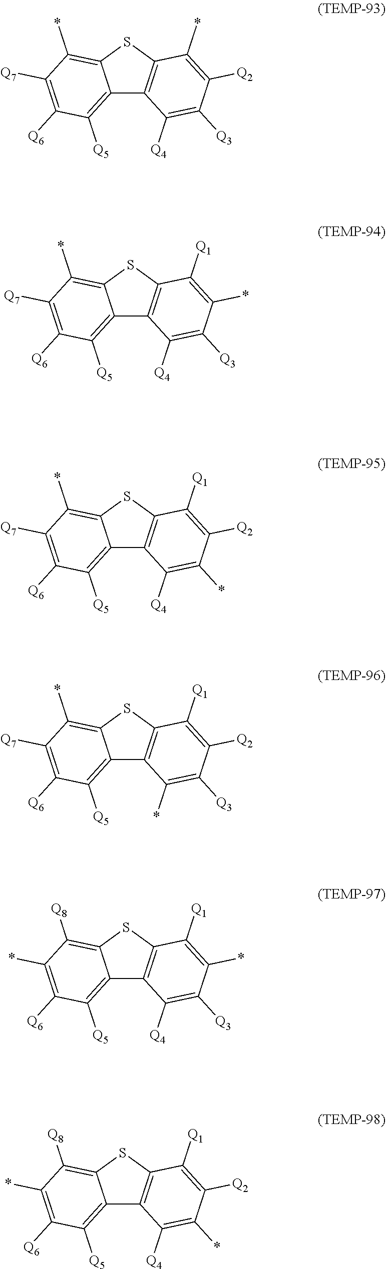

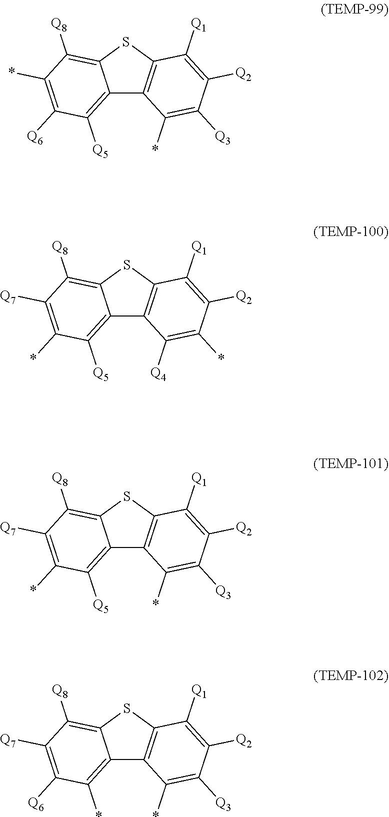

In the formulae (TEMP-83) to (TEMP-102), Q₁ to Q₈ each independently are a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring,” at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

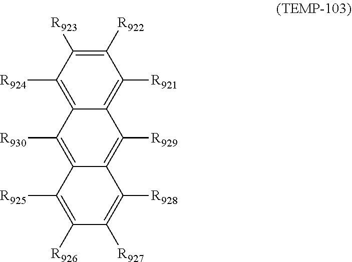

For instance, when “at least one combination of” adjacent two or more of R₉₂₁ to R₉₃₀ “are mutually bonded to form a ring,” the pair of adjacent ones of R₉₂₁ to R₉₃₀ (i.e. the combination at issue) is a pair of R₉₂₁ and a pair of R₉₂₂, R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, or a pair of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring QA and R₉₂₅ and R₉₂₆ are simultaneously mutually bonded to form a ring Q_{B}, the anthracene compound represented by the formula (TEMP-103) is represented by a formula (TEMP-104) below.

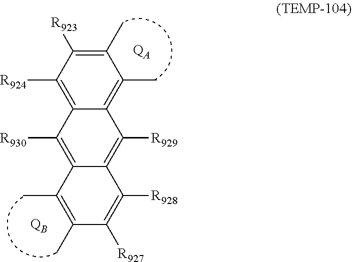

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂, R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

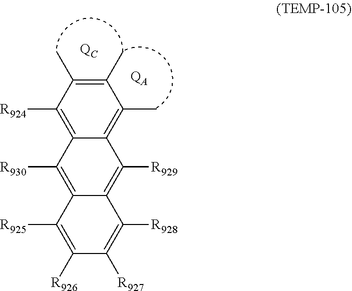

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formulae (TEMP-104) and (TEMP-105) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocyclic ring include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded with R₉₂₁, a carbon atom of the anthracene skeleton bonded with R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded with R₉₂₁, a carbon atom of the anthracene skeleton bonded with R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituents Mentioned Herein.”

When the “saturated ring” or the “unsaturated ring” has a substituent, the substituent is, for instance, the substituent described in later-described “optional substituent.” When the “monocyclic ring” or the “fused ring” has a substituent, specific examples of the substituent are the substituents described in the above under the subtitle “Substituents Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance “bonded to form a ring.”

### Substituent Meant by “Substituted or Unsubstituted”

In an exemplary embodiment herein, the substituent meant by the phrase “substituted or unsubstituted” (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms.

Herein, R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

When two or more R₉₀₁ are present, the two or more R₉₀₁ are mutually the same or different.

When two or more R₉₀₂ are present, the two or more R₉₀₂ are mutually the same or different.

When two or more R₉₀₃ are present, the two or more R₉₀₃ are mutually the same or different.

When two or more R₉₀₄ are present, the two or more R₉₀₄ are mutually the same or different.

When two or more R₉₀₅ are present, the two or more R₉₀₅ are mutually the same or different.

When two or more R₉₀₆ are present, the two or more R₉₀₆ are mutually the same or different.

When two or more R₉₀₇ are present, the two or more R₉₀₇ are mutually the same or different:

In an exemplary embodiment, the substituent meant by “substituted or unsubstituted” is selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent meant by “substituted or unsubstituted” is selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted saturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represents a range whose lower limit is the value (AA) recited before “to” and whose upper limit is the value (BB) recited after “to.”

### First Exemplary Embodiment

An arrangement of an organic EL device according to the first exemplary embodiment of the invention will be described.

The organic EL device includes an organic layer between both electrodes of an anode and a cathode. The organic layer includes at least one layer formed of an organic compound. Alternatively, the organic layer includes a plurality of layers formed of an organic compound(s). The organic layer may further include an inorganic compound. In the organic EL device according to the exemplary embodiment, at least one layer of the organic layer is an emitting layer. Therefore, for instance, the organic layer may consist of a single emitting layer or, alternatively, may further include at least one layer usable for an organic EL device. Examples of the layer usable in the organic EL device, which are not particularly limited, include at least one layer selected from the group consisting of a hole injecting layer, hole transporting layer, electron injecting layer, electron transporting layer, and blocking layer.

An organic EL device in the exemplary embodiment includes an anode, a cathode, and an emitting layer provided between the anode and the cathode, in which the emitting layer includes a delayed fluorescent compound M2 represented by a formula (2) below and a compound M3 represented by a formula (3) below, and a singlet energy S₁(M2) of the compound M2 and a singlet energy S₁(M3) of the compound M3 satisfy a relationship of a numerical formula (Numerical Formula 1) below.

S₁(M3)>S₁(M2) Numerical Formula 1

In the delayed fluorescent compound M2 represented by the formula (2), a conjugation length is extended by substitution with a group represented by a formula (21) (e.g., a phenyl group), thereby stabilizing an excited state. The excited state of the delayed fluorescent compound M2 in the emitting layer is thus stabilized, thereby increasing luminous efficiency of the organic EL device.

The inventors have found that a charge balance is appropriately adjusted and thus the luminous efficiency is further improved by containing the compound M2 and the compound M3 represented by the formula (3) in combination in the emitting layer.

In the exemplary embodiment, the compound M2 is preferably a dopant material (sometimes referred to as a guest material, emitter, or luminescent material) and the compound M3 is preferably a host material (sometimes referred to as a matrix material).

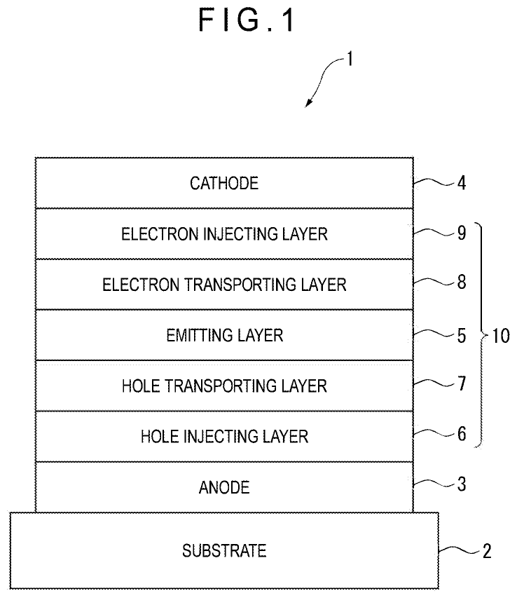

FIG. 1 schematically shows an exemplary arrangement of an organic EL device in the exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, hole transporting layer 7, emitting layer 5, electron transporting layer 8, and electron injecting layer 9, which are sequentially laminated on the anode 3.

In an exemplary arrangement of the exemplary embodiment, the emitting layer may contain a metal complex.

In another exemplary arrangement of the exemplary embodiment, the emitting layer preferably does not contain a phosphorescent material (phosphorescent dopant material).

In still another exemplary arrangement of the exemplary embodiment, the emitting layer preferably does not contain a heavy metal complex and a phosphorescent rare-earth metal complex. Examples of the heavy metal complex herein include an iridium complex, osmium complex, and platinum complex.

In a further exemplary arrangement of the exemplary embodiment, the emitting layer also preferably does not contain a phosphorescent metal complex and also preferably does not contain a metal complex.

### Compound M2

The emitting layer of the organic EL device in the exemplary embodiment contains the compound M2 represented by the formula (2) below. The compound M2 in the exemplary embodiment is a thermally activated delayed fluorescent compound.

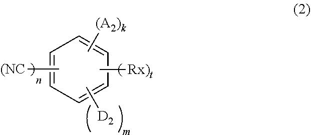

In the formula (2):

k is 1, 2, 3, or 4;

m is 1, 2, 3, or 4;

n is 1 or 2;

t is 0, 1, 2, or 3;

k+m+n+t is 6;

when t is 2 or 3, a plurality of Rx are mutually the same or different;

A₂ is a group represented by a formula (21) below;

when k is 2, 3, or 4, a plurality of A₂ are mutually the same or different;

D₂ is a group represented by a formula (22) below;

when m is 2, 3, or 4, a plurality of D₂ are mutually the same or different; and CN is a cyano group.

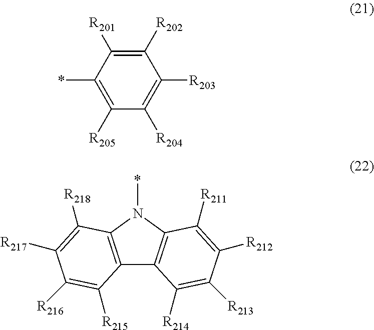

In the formula (21): at least one combination of adjacent two or more of R₂₀₁ to R₂₀₅ are mutually bonded to form a substituted or unsubstituted monocyclic ring, mutually bonded to form a substituted or unsubstituted fused ring, or not mutually bonded.

In the formula (22): at least one combination of adjacent two or more of R₂₁₁ to R₂₁₈ are mutually bonded to form a substituted or unsubstituted monocyclic ring, mutually bonded to form a substituted or unsubstituted fused ring, or not mutually bonded.

* in each of the formulae (21) and (22) represents a bonding position to a benzene ring in the formula (2).

Rx in the formula (2), R₂₀₁ to R₂₀₅ not forming the substituted or unsubstituted monocyclic ring and not forming the substituted or unsubstituted fused ring in the formula (21), and R₂₁₁ to R₂₁₈ not forming the substituted or unsubstituted monocyclic ring and not forming the substituted or unsubstituted fused ring in the formula (22) are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms.

In the compound M2 and the compound M3:

R₉₀₁, R₉₀₂, R₉₀₃, R₉₀₄, R₉₀₅, R₉₀₆, R₉₀₇, R₉₀₈, R₉₀₉, R₉₃₁, R₉₃₂, R₉₃₃, R₉₃₄, R₉₃₅, R₉₃₆, and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₉₀₁ are present, the plurality of R₉₀₁ are mutually the same or different;

when a plurality of R₉₀₂ are present, the plurality of R₉₀₂ are mutually the same or different;

when a plurality of R₉₀₃ are present, the plurality of R₉₀₃ are mutually the same or different;

when a plurality of R₉₀₄ are present, the plurality of R₉₀₄ are mutually the same or different;

when a plurality of R₉₀₅ are present, the plurality of R₉₀₅ are mutually the same or different;

when a plurality of R₉₀₆ are present, the plurality of R₉₀₆ are mutually the same or different;

when a plurality of R₉₀₇ are present, the plurality of R₉₀₇ are mutually the same or different;

when a plurality of R₉₀₈ are present, the plurality of R₉₀₈ are mutually the same or different;

when a plurality of R₉₀₉ are present, the plurality of R₉₀₉ are mutually the same or different;

when a plurality of R₉₃₁ are present, the plurality of R₉₃₁ are mutually the same or different;

when a plurality of R₉₃₂ are present, the plurality of R₉₃₂ are mutually the same or different;

when a plurality of R₉₃₃ are present, the plurality of R₉₃₃ are mutually the same or different;

when a plurality of R₉₃₄ are present, the plurality of R₉₃₄ are mutually the same or different;

when a plurality of R₉₃₅ are present, the plurality of R₉₃₅ are mutually the same or different;

when a plurality of R₉₃₆ are present, the plurality of R₉₃₆ are mutually the same or different; and

when a plurality of R₉₃₇ are present, the plurality of R₉₃₇ are mutually the same or different.

n in the formula (2) is preferably 2. The compound M2 is also preferably a dicyanobenzene compound in which two cyano groups are bonded to a benzene ring.

The compound M2 is also preferably a compound represented by a formula (201) below.

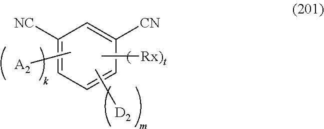

In the formula (201): A₂, D₂ and Rx respectively represent the same as A₂, D₂ and Rx in the formula (2);

k is 1, 2, or 3;

m is 1, 2, or 3;

t is 0, 1, or 2; and

k+m+t is 4.

The compound M2 is also preferably a compound represented by a formula (210) or a formula (230) below.

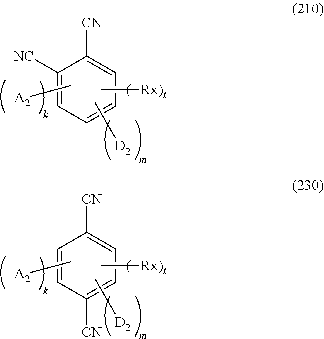

In the formulae (210) and (230): A₂, D₂ and Rx respectively represent the same as A₂, D₂ and Rx in the formula (2);

k is 1, 2, or 3;

m is 1, 2, or 3;

t is 0, 1, or 2; and

k+m+t is 4.

m in the compound M2 is preferably 2.

The compound M2 is also preferably a compound represented by a formula (211) below.

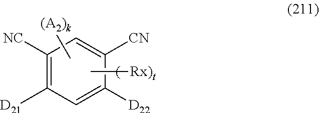

In the formula (211):

D₂₁ and D₂₂ each independently represent the same as D₂;

A₂ and Rx respectively represent the same as A₂ and Rx in the formula (2);

k is 1 or 2;

t is 0 or 1; and

k+t is 2.

In the compound M2, D₂₁ and D₂₂ are mutually the same or different.

In the compound M2, k is preferably 1 or 2, more preferably 2.

The compound M2 is also preferably a compound represented by a formula (202) or a formula (203) below.

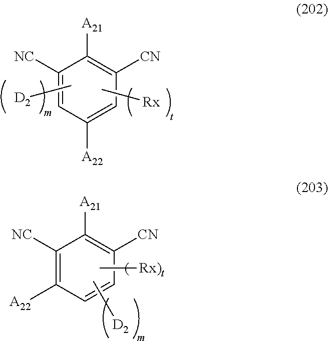

In the formula (202) or (203):

A₂₁ and A₂₂ each independently represent the same as A₂;

D₂ and Rx respectively represent the same as D₂ and Rx in the formula (2);

m is 1 or 2;

t is 0 or 1; and

m+t is 2.

In the compound M2, A₂₁ and A₂₂ are mutually the same or different.

The compound M2 is also preferably a compound represented by a formula (221) below.

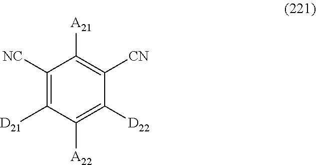

In the formula (221):

A₂₁ and A₂₂ each independently represent the same as A₂; and

D₂₁ and D₂₂ each independently represent the same as D₂.

The compound M2 is also preferably a compound represented by a formula (222) below.

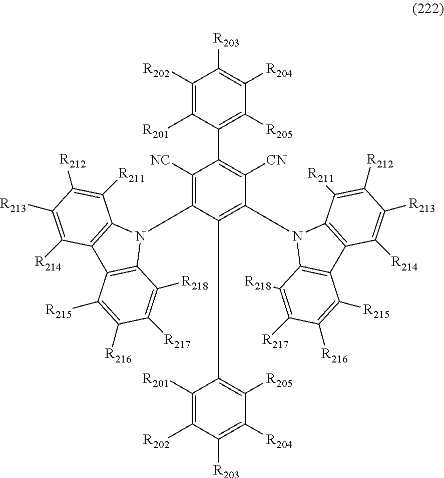

In the formula (222), R₂₀₁ to R₂₀₅ each independently represent the same as R₂₀₁ to R₂₀₅ in the formula (21), and R₂₁₁ to R₂₁₈ each independently represent the same as R₂₁₁ to R₂₁₈ in the formula (22).

In the compound M2, a plurality of R₂₀₁ are mutually the same or different, a plurality of R₂₀₂ are mutually the same or different, a plurality of R₂₀₃ are mutually the same or different, a plurality of R₂₀₄ are mutually the same or different, a plurality of R₂₀₅ are mutually the same or different, a plurality of R₂₁₁ are mutually the same or different, a plurality of R₂₁₂ are mutually the same or different, a plurality of R₂₁₃ are mutually the same or different, a plurality of R₂₁₄ are mutually the same or different, a plurality of R₂₁₅ are mutually the same or different, a plurality of R₂₁₆ are mutually the same or different, a plurality of R₂₁₇ are mutually the same or different, and a plurality of R₂₁₈ are mutually the same or different.

In the compound M2: Rx; R₂₀₁ to R₂₀₅ not forming the substituted or unsubstituted monocyclic ring and not forming the substituted or unsubstituted fused ring; and R₂₁₁ to R₂₁₈ not forming the substituted or unsubstituted monocyclic ring and not forming the substituted or unsubstituted fused ring are each independently preferably a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M2: Rx; R₂₀₁ to R₂₀₅ not forming the substituted or unsubstituted monocyclic ring and not forming the substituted or unsubstituted fused ring; and R₂₁₁ to R₂₁₈ not forming the substituted or unsubstituted monocyclic ring and not forming the substituted or unsubstituted fused ring are each independently preferably a hydrogen atom, or a substituted or unsubstituted aryl group having 6 to 50 ring carbon atoms.

In the compound M2: Rx; R₂₀₁ to R₂₀₅ not forming the substituted or unsubstituted monocyclic ring and not forming the substituted or unsubstituted fused ring; and R₂₁₁ to R₂₁₈ not forming the substituted or unsubstituted monocyclic ring and not forming the substituted or unsubstituted fused ring are each preferably a hydrogen atom.

In the compound M2, A₂ is preferably any one group selected from the group consisting of groups represented by formulae (A21) to (A25) below.

In the compound M2, A₂₁ and A₂₂ are each independently preferably any one group selected from the group consisting of groups represented by the formulae (A21) to (A25) below.

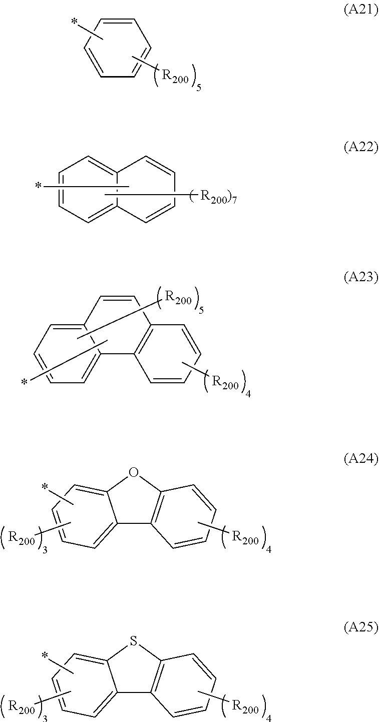

In the formulae (A21) to (A25):

at least one combination of adjacent two or more of a plurality of R₂₀₀ are mutually bonded to form a substituted or unsubstituted monocyclic ring, mutually bonded to form a substituted or unsubstituted fused ring, or not mutually bonded;

R₂₀₀ not forming the substituted or unsubstituted monocyclic ring and not forming the substituted or unsubstituted fused ring is each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms; and

* in each of the formulae (A21) to (A25) represents a bonding position to a benzene ring in the formula (2).

In the compound M2, A₂ is preferably any one group selected from the group consisting of groups represented by the formulae (A21), (A24) and (A25).

In the compound M2, A₂₁ and A₂₂ are each independently preferably any one group selected from the group consisting of groups represented by the formulae (A21), (A24) and (A25).

A₂ in the compound M2 is preferably a group represented by the formula (A21).

A₂₁ and A₂₂ in the compound M2 are preferably each a group represented by the formula (A21).

In each of combinations of adjacent two or more of a plurality of R₂₀₀ in the compound M2, R₂₀₀ are also preferably not bonded to each other.

A₂ in the compound M2 is preferably a group represented by the formula (A21) in which R₂₀₀ is a hydrogen atom.

A₂₁ and A₂₂ in the compound M2 are preferably a group represented by the formula (A21) in which R₂₀₀ is a hydrogen atom.

In the formulae (A21) to (A25): R₂₀₀ is each independently preferably a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the formulae (A21) to (A25): R₂₀₀ is each independently preferably a hydrogen atom, or a substituted or unsubstituted aryl group having 6 to 50 ring carbon atoms.

In the formulae (A21) to (A25): R₂₀₀ is preferably a hydrogen atom.

In the compound M2, D₂ is preferably any one group selected from the group consisting of groups represented by formulae (B21) to (B23) below.

In the compound M2, D₂₁ and D₂₂ are each independently preferably any one group selected from the group consisting of groups represented by the formulae (B21) to (B23) below.

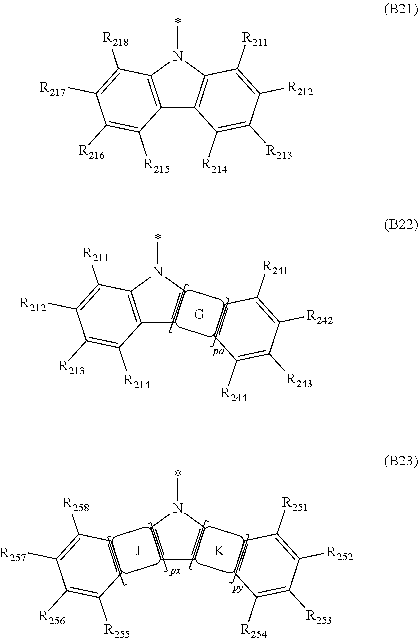

In the formula (B22), at least one combination of adjacent two or more of R₂₁₁ to R₂₁₄ and R₂₄₁ to R₂₄₄ are mutually bonded to form a substituted or unsubstituted monocyclic ring, mutually bonded to form a substituted or unsubstituted fused ring, or not mutually bonded.

In the formula (B23), at least one combination of adjacent two or more of R₂₅₁ to R₂₅₈ are mutually bonded to form a substituted or unsubstituted monocyclic ring, mutually bonded to form a substituted or unsubstituted fused ring, or not mutually bonded.

R₂₁₁ to R₂₁₈ in the formula (B21), R₂₁₁ to R₂₁₄ and R₂₄₁ to R₂₄₄ not forming the substituted or unsubstituted monocyclic ring and not forming the substituted or unsubstituted fused ring in the formula (B22), and R₂₅₁ to R₂₅₈ not forming the substituted or unsubstituted monocyclic ring and not forming the substituted or unsubstituted fused ring in the formula (B23) are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms.

In the formulae (B22) and (B23):

a ring G, a ring J, and a ring K are each independently any one cyclic structure selected from the group consisting of cyclic structures represented by formulae (B24) and (B25) below;

the ring G, the ring J, and the ring K are each fused with adjacent rings at any positions;

pa, px, and py are each independently 1, 2, 3, or 4;

when pa is 2, 3, or 4, a plurality of rings G are mutually the same or different;

when px is 2, 3, or 4, a plurality of rings J are mutually the same or different;

when py is 2, 3, or 4, a plurality of rings K are mutually the same or different; and

* in each of the formulae (B21) to (B23) represents a bonding position to a benzene ring in the formula (2).

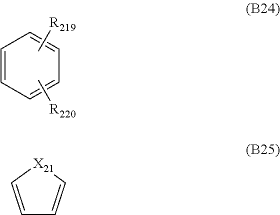

In the formula (B24):

a combination of R₂₁₉ and R₂₂₀ are mutually bonded to form a substituted or unsubstituted monocyclic ring, mutually bonded to form a substituted or unsubstituted fused ring, or not mutually bonded.

In the formula (B25):

X₂₁ is a sulfur atom, an oxygen atom, NR₂₆₁, or CR₂₆₂R₂₆₃; and a combination of R₂₆₂ and R₂₆₃ are mutually bonded to form a substituted or unsubstituted monocyclic ring, mutually bonded to form a substituted or unsubstituted fused ring, or not mutually bonded.

R₂₆₁; R₂₁₉ and R₂₂₀ not forming the substituted or unsubstituted monocyclic ring and not forming the substituted or unsubstituted fused ring; and R₂₆₂ and R₂₆₃ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms.

In the formula (B21), each combination of combinations of adjacent two or more of R₂₁₁ to R₂₁₈ are not mutually bonded.

In the compound M2: it is preferable that R₂₁₁ to R₂₁₈ in the formula (B21), R₂₁₁ to R₂₁₄ and R₂₄₁ to R₂₄₄ in the formula (B22), R₂₅₁ to R₂₅₈ in the formula (B23), and R₂₁₉ and R₂₂₀ in the formula (B24) are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M2: it is preferable that R₂₁₁ to R₂₁₈ in the formula (B21), R₂₁₁ to R₂₁₄ and R₂₄₁ to R₂₄₄ in the formula (B22), R₂₅₁ to R₂₅₈ in the formula (B23), and R₂₁₉ and R₂₂₀ in the formula (B24) are each independently a hydrogen atom, or a substituted or unsubstituted aryl group having 6 to 50 ring carbon atoms.

In the compound M2: it is preferable that R₂₁₁ to R₂₁₈ in the formula (B21), R₂₁₁ to R₂₁₄ and R₂₄₁ to R₂₄₄ in the formula (B22), R₂₅₁ to R₂₅₈ in the formula (B23), and R₂₁₉ and R₂₂₀ in the formula (B24) are each a hydrogen atom.

In the compound M2, R₂₆₁ is preferably a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M2, it is preferable that a combination of R₂₆₂ and R₂₆₃ are mutually bonded to form a substituted or unsubstituted monocyclic ring, mutually bonded to form a substituted or unsubstituted fused ring, or not mutually bonded;

R₂₆₂ and R₂₆₃ not forming the substituted or unsubstituted monocyclic ring and not forming the substituted or unsubstituted fused ring are each independently a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M2: it is preferable that the formula (B22) represents any one cyclic structure selected from the group consisting of cyclic structures represented by formulae (a1) to (a6) below;

px and py in the formula (B23) are 2; and at least one of the rings J is a cyclic structure represented by the formula (B25) and at least one of the rings K is a cyclic structure represented by the formula (B25).

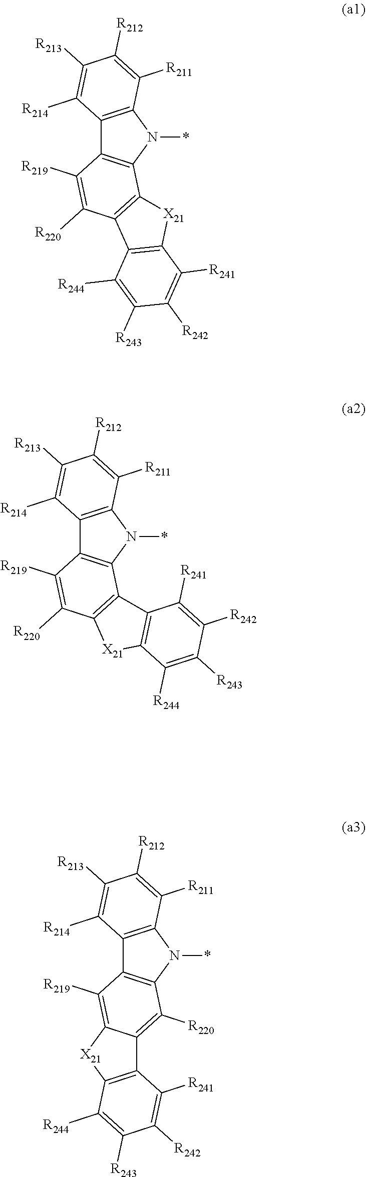

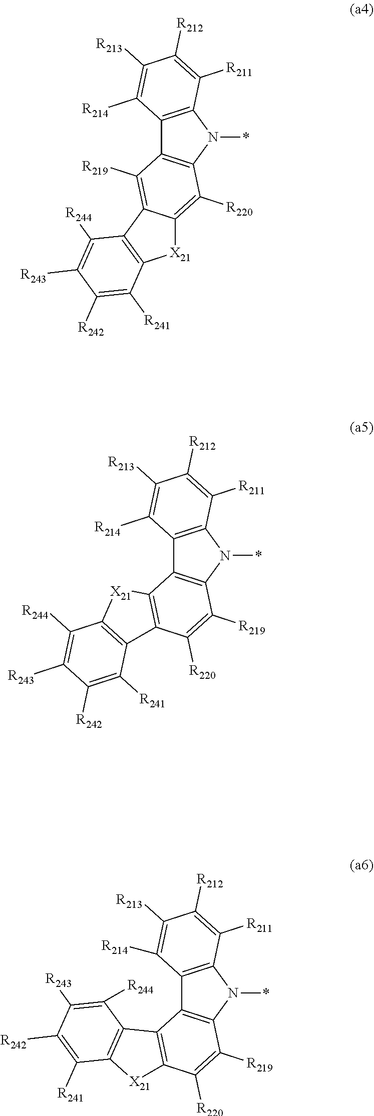

In the formulae (a1) to (a6):

R₂₁₁ to R₂₁₄ and R₂₄₁ to R₂₄₄ respectively represent the same as R₂₁₁ to R₂₁₄ and R₂₄₁ to R₂₄₄ in the formula (B22);

X₂₁, R₂₁₉ and R₂₂₀ respectively represent the same as X₂₁, R₂₁₉ and R₂₂₀ in the formula (B25); and

* in each of the formulae (a1) to (a6) represents a bonding position to a benzene ring in the formula (2).

D₂ in the compound M2 is preferably represented by the formula (B22) or (B23).

D₂₁ and D₂₂ in the compound M2 are each independently preferably a group represented by the formula (B22) or (B23).

X₂₁ in the compound M2 is preferably a sulfur atom, an oxygen atom, or C R₂₆₂R₂₆₃.

X₂₁ in the compound M2 is preferably a sulfur atom or an oxygen atom.

In the compound M2, it is preferable that a substituent for the “substituted or unsubstituted” group is an unsubstituted alkyl group having 1 to 25 carbon atoms, an unsubstituted alkenyl group having 2 to 25 carbon atoms, an unsubstituted alkynyl group having 2 to 25 carbon atoms, an unsubstituted cycloalkyl group having 3 to 25 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), an unsubstituted aralkyl group having 7 to 50 carbon atoms, a group represented by —C(═O)R₉₀₈, a group represented by —COOR₉₀₉, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a group represented by —S(═O)₂R₉₃₈, a halogen atom, a cyano group, a nitro group, an unsubstituted aryl group having 6 to 25 ring carbon atoms, or an unsubstituted heterocyclic group having 5 to 25 ring atoms; and

R₉₀₁ to R₉₀₉ and R₉₃₁ to R₉₃₈ are each independently a hydrogen atom, an unsubstituted alkyl group having 1 to 25 carbon atoms, an unsubstituted aryl group having 6 to 25 ring carbon atoms, or an unsubstituted heterocyclic group having 5 to ring atoms.

In the compound M2, it is preferable that a substituent for the “substituted or unsubstituted” group is a halogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compound M2, it is preferable that a substituent for the “substituted or unsubstituted” group is an unsubstituted alkyl group having 1 to 10 carbon atoms, an unsubstituted aryl group having 6 to 12 ring carbon atoms, or an unsubstituted heterocyclic group having 5 to 12 ring atoms.

In the compound M2, it is also preferable that all of the “substituted or unsubstituted” groups are “unsubstituted” groups.

Herein, a group represented by —O—(R₉₀₄) in which R₉₀₄ is a hydrogen atom is a hydroxy group.

Herein, a group represented by —S—(R₉₀₅) in which R₉₀₅ is a hydrogen atom is a thiol group.

Herein, a group represented by —P(═O)(R₉₃₁)(R₉₃₂) in which R₉₃₁ and R₉₃₂ are each a substituent is a substituted phosphine oxide group and a group represented by —P(═O)(R₉₃₁)(R₉₃₂) in which R₉₃₁ and R₉₃₂ are each an aryl group is an arylphosphoryl group.

Herein, a group represented by —Ge(R₉₃₃)(R₉₃₄)(R₉₃₅) in which R₉₃₃, R₉₃₄ and R₉₃₅ are each a substituent is a substituted germanium group.

Herein, a group represented by —B(R₉₃₆)(R₉₃₇) in which R₉₃₆ and R₉₃₇ are each a substituent is a substituted boryl group.

### Delayed Fluorescence

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a mechanism of generating delayed fluorescence is explained in FIG. 10.38 in the document. The compound M2 in the exemplary embodiment is preferably a compound exhibiting thermally activated delayed fluorescence generated by such a mechanism.

In general, emission of delayed fluorescence can be confirmed by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

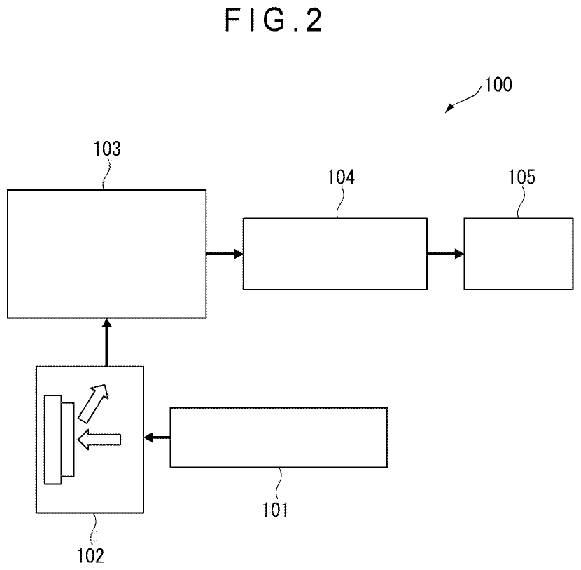

FIG. 2 shows a schematic diagram of an exemplary device for measuring the transient PL. An example of a method of measuring a transient PL using FIG. 2 and an example of behavior analysis of delayed fluorescence will be described.

A transient PL measuring device 100 in FIG. 2 includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. A device for measuring the transient PL is not limited to the device described in the exemplary embodiment.

The sample to be housed in the sample chamber 102 is obtained by doping a matrix material with a doping material at a concentration of 12 mass % and forming a thin film on a quartz substrate.

The thin film sample housed in the sample chamber 102 is irradiated with the pulse laser from the pulse laser 101 to excite the doping material. Emission is extracted in a direction of 90 degrees with respect to a radiation direction of the excited light. The extracted emission is divided by the spectrometer 103 to form a two-dimensional image in the streak camera 104. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was manufactured as described above from a reference compound H1 as the matrix material and a reference compound D₁ as the doping material and was measured in terms of the transient PL.

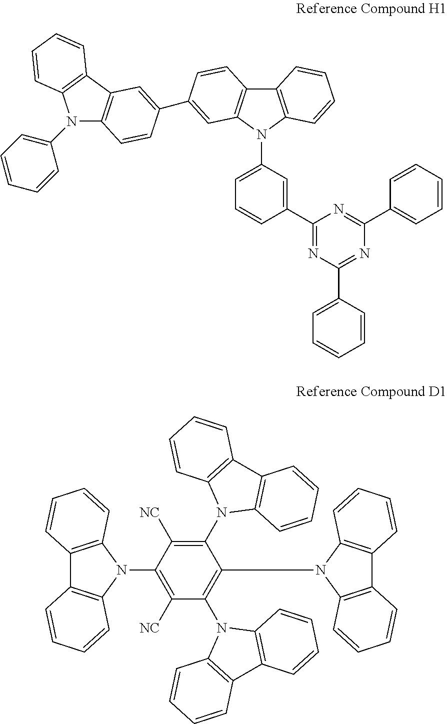 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

The decay curve was analyzed with respect to the above thin film sample A and a thin film sample B. The thin film sample B was manufactured in the same manner as described above from a reference compound H2 as the matrix material and the reference compound D₁ as the doping material.

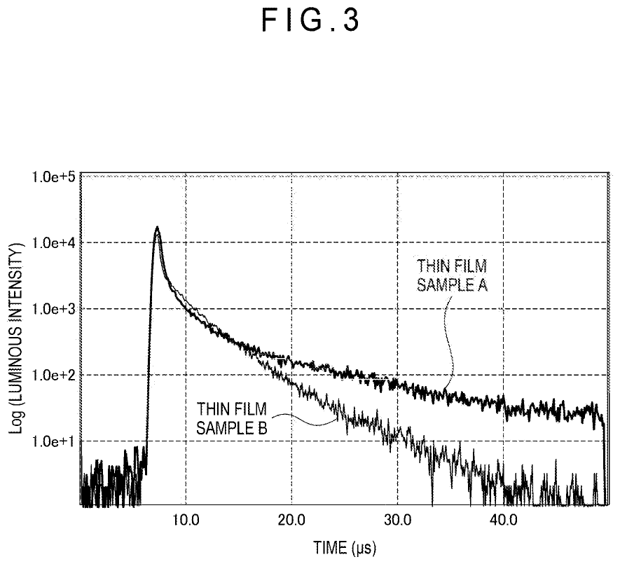

FIG. 3 shows decay curves obtained from transient PL obtained by measuring the thin film samples A and B.

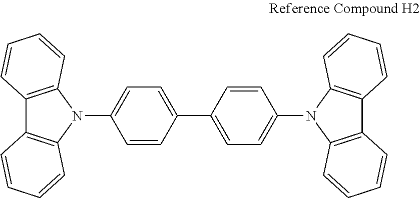 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by inverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

Herein, a sample manufactured by the following method is used for measuring delayed fluorescence of the compound M2. For instance, the compound M2 is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (manufactured by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

In the exemplary embodiment, provided that an amount of Prompt emission of a measurement target compound (compound M2) is denoted by X_{P} and the amount of Delay emission is denoted by X_{D}, a value of X_{D}/X_{P} is preferably 0.05 or more.

The amounts of Prompt emission and Delay emission and a ratio of the amounts thereof in compounds other than the compound M2 herein are measured in the same manner as those of the compound M2.

### ΔST

In the exemplary embodiment, a difference (S₁-T_{77K}) between the lowest singlet energy Si and an energy gap T_{77K} at 77K is defined as ΔST.

A difference ΔST(M2) between the lowest singlet energy S₁(M2) of the compound M2 and an energy gap T_{77K}(M2) at 77K of the compound M2 is preferably less than 0.3 eV, more preferably less than 0.2 eV, further preferably less than 0.1 eV, more further preferably less than 0.01 eV. In other words, ΔST(M2) preferably satisfies a relationship of a numerical formula ((Numerical Formula 10), (Numerical Formula 11), (Numerical Formula 12) or (Numerical Formula 13)) below.

ΔST(M2)=S₁(M2)−T_{77K}(M2)<0.3 eV (Numerical Formula 10)

ΔST(M2)=S₁(M2)−T_{77K}(M2)<0.2 eV (Numerical Formula 11)

ΔST(M2)=S₁(M2)−T_{77K}(M2)<0.1 eV (Numerical Formula 12)

ΔST(M2)=S₁(M2)−T_{77K}(M2)<0.01 eV (Numerical Formula 13)

### Relationship Between Triplet Energy and Energy Gap at 77K

Here, a relationship between a triplet energy and an energy gap at 77 [K] will be described. In the exemplary embodiment, the energy gap at 77K is different from a typical triplet energy in some aspects.

The triplet energy is measured as follows. Firstly, a solution in which a compound (measurement target) is dissolved in an appropriate solvent is encapsulated in a quartz glass tube to prepare a sample. A phosphorescent spectrum (ordinate axis: phosphorescent luminous intensity, abscissa axis: wavelength) of the sample is measured at a low temperature (77K). A tangent is drawn to the rise of the phosphorescent spectrum close to the short-wavelength region. The triplet energy is calculated by a predetermined conversion equation based on a wavelength value at an intersection of the tangent and the abscissa axis.

Herein, the delayed fluorescent compound used in the present exemplary embodiment is preferably a compound having a small ΔST. When ΔST is small, intersystem crossing and inverse intersystem crossing are likely to occur even at a low temperature (77K), so that the singlet state and the triplet state coexist. As a result, the spectrum to be measured in the same manner as the above includes emission from both the singlet state and the triplet state. Although it is difficult to distinguish the emission from the singlet state from the emission from the triplet state, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is encapsulated in a quartz cell to provide a measurement sample. A phosphorescent spectrum (ordinate axis: phosphorescent luminous intensity, abscissa axis: wavelength) of the sample is measured at a low temperature (77K). A tangent is drawn to the rise of the phosphorescent spectrum close to the short-wavelength region. An energy amount is calculated by a conversion equation below based on a wavelength value λ_{edge} [nm] at an intersection of the tangent and the abscissa axis and is defined as an energy gap T_{77K} at 77K.

T_{77K} [eV]=1239.85/λedge Conversion Equation (F1):

The tangent to the rise of the phosphorescence spectrum close to the short-wavelength region is drawn as follows. While moving on a curve of the phosphorescence spectrum from the short-wavelength region to the maximum spectral value closest to the short-wavelength region among the maximum spectral values, a tangent is checked at each point on the curve toward the long-wavelength of the phosphorescence spectrum. An inclination of the tangent is increased along the rise of the curve (i.e., a value of the ordinate axis is increased). A tangent drawn at a point of the maximum inclination (i.e., a tangent at an inflection point) is defined as the tangent to the rise of the phosphorescence spectrum close to the short-wavelength region.

The maximum with peak intensity being 15% or less of the maximum peak intensity of the spectrum is not included in the above-mentioned maximum closest to the short-wavelength region. The tangent drawn at a point of the maximum spectral value being closest to the short-wavelength region and having the maximum inclination is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Lowest Singlet Energy S1

A method of measuring the lowest singlet energy Si with use of a solution (occasionally referred to as a solution method) is exemplified by a method below.

A toluene solution in which a measurement target compound is dissolved at a concentration of 10 μmol/L is prepared and is encapsulated in a quartz cell to provide a measurement sample. Absorption spectrum (ordinate axis: absorption intensity, abscissa axis: wavelength) of the sample is measured at the normal temperature (300K). A tangent is drawn to the fall of the absorption spectrum on the long-wavelength side, and a wavelength value Kedge (nm) at an intersection of the tangent and the abscissa axis is assigned to a conversion equation (F2) below to calculate the lowest singlet energy.

S₁ [eV]=1239.85/λedge Conversion Equation (F2):

Any device for measuring absorption spectrum is usable. For instance, a spectrophotometer (U3310 manufactured by Hitachi, Ltd.) is usable.

The tangent to the fall of the absorption spectrum on the long-wavelength side is drawn as follows. While moving on a curve of the absorption spectrum from the maximum spectral value closest to the long-wavelength side in a long-wavelength direction, a tangent at each point on the curve is checked. An inclination of the tangent is decreased and increased in a repeated manner as the curve falls (i.e., a value of the ordinate axis is decreased). A tangent drawn at a point of the minimum inclination closest to the long-wavelength side (except when absorbance is 0.1 or less) is defined as the tangent to the fall of the absorption spectrum on the long-wavelength side.

The maximum absorbance of 0.2 or less is not included in the above-mentioned maximum absorbance on the long-wavelength side.

### Manufacturing Method of Compound M2

The compound M2 according to the exemplary embodiment can be manufactured by application of known substitution reactions and materials depending on a target compound, in accordance with or based on synthesis methods described later in Examples.

### Specific Examples of Compound M2

Specific examples of the compound M2 in the exemplary embodiment include compounds as follows. It should however be noted that the invention is not limited to the specific examples of the compound. Herein, a deuterium atom is denoted by D and a protium atom is denoted by H or a description for a protium is omitted.

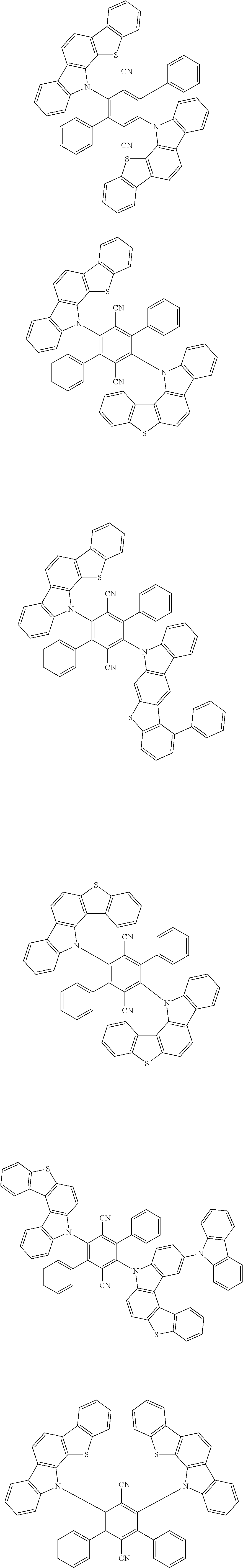

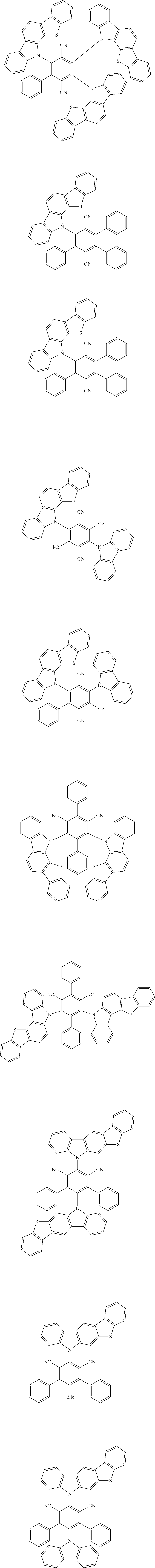

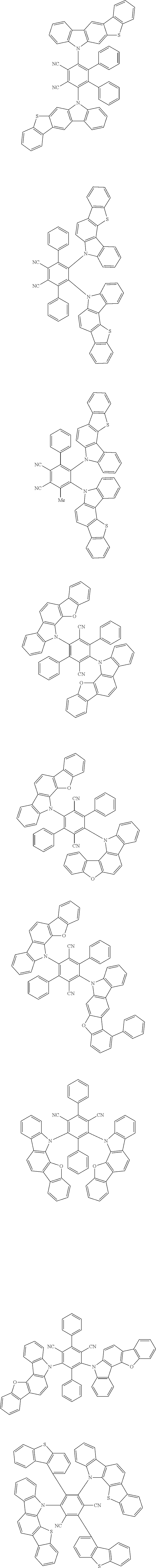

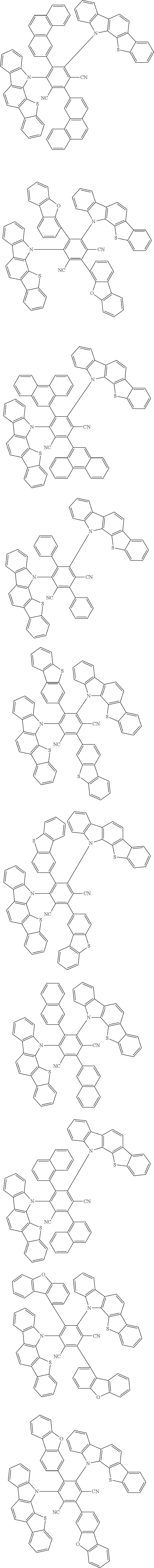

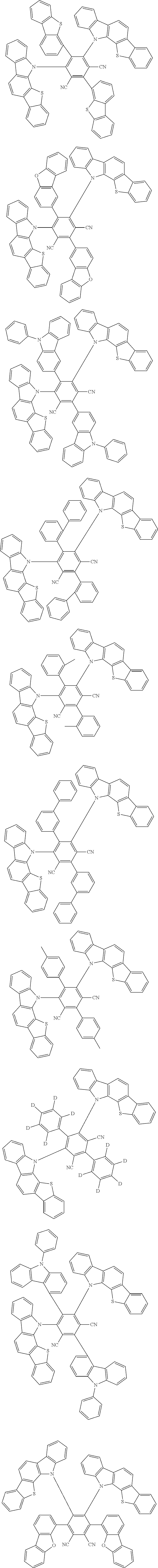

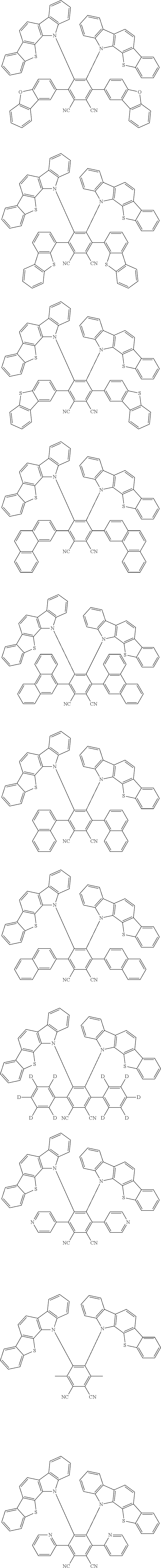

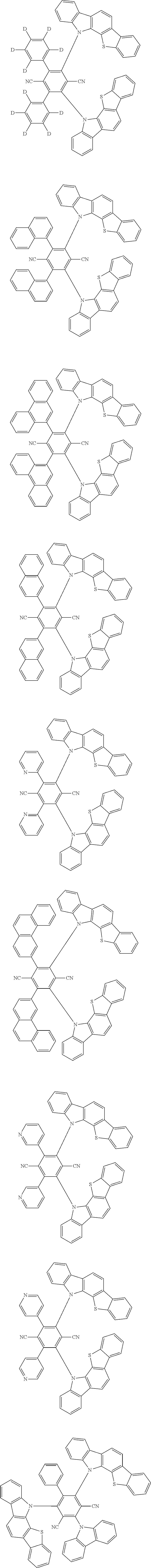

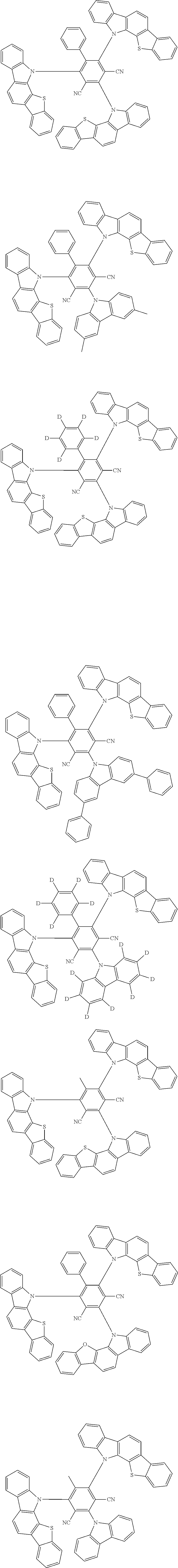

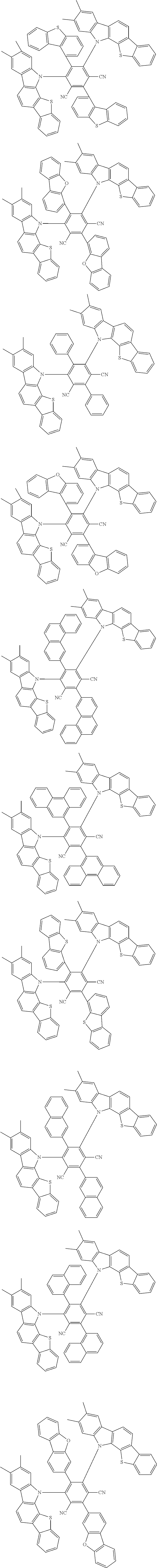

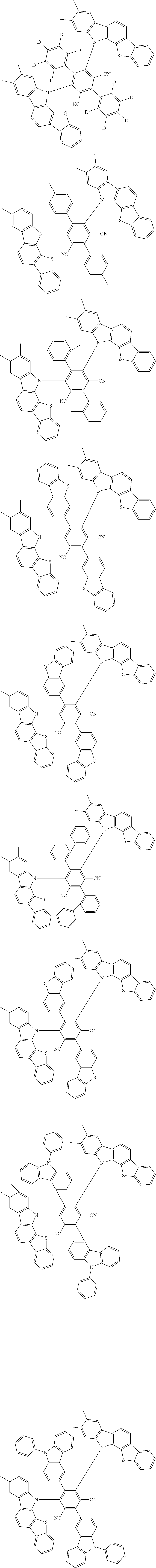

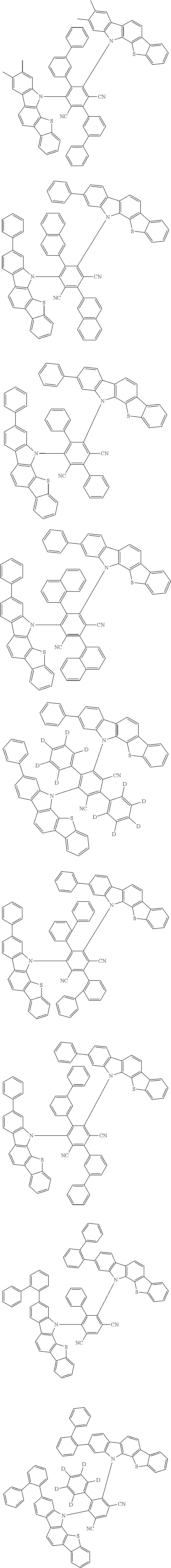

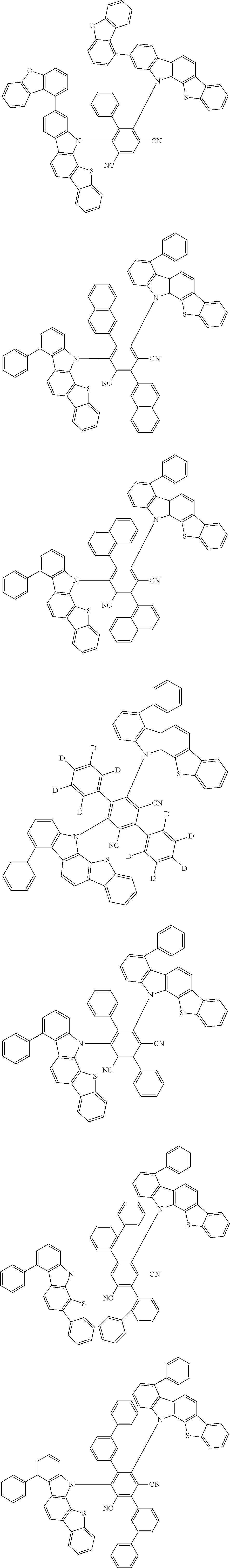

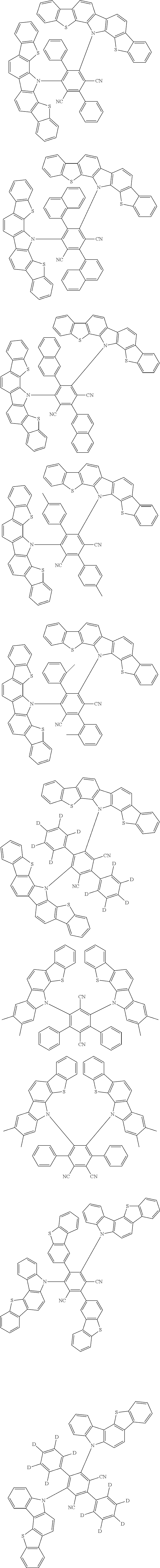

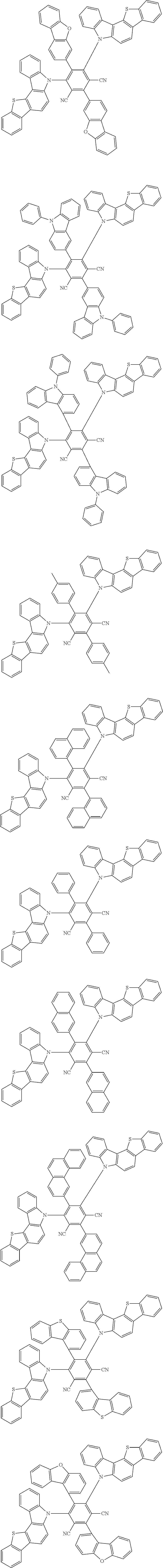

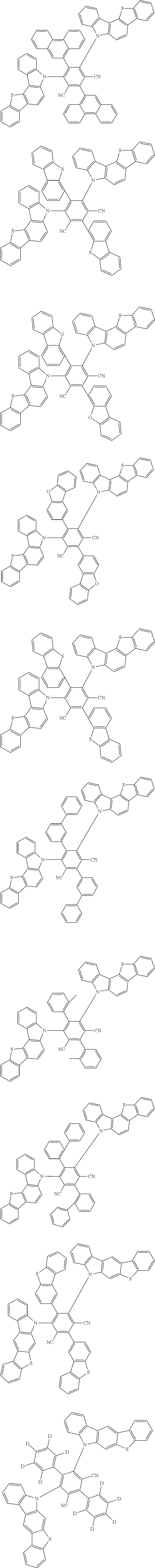

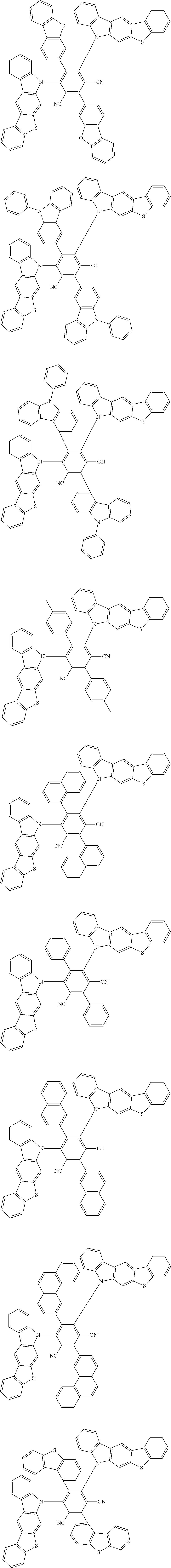

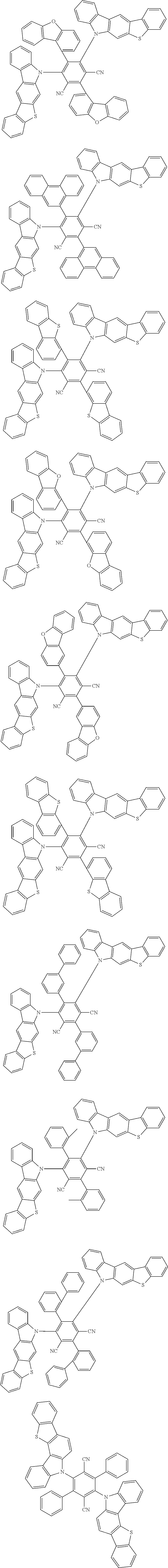

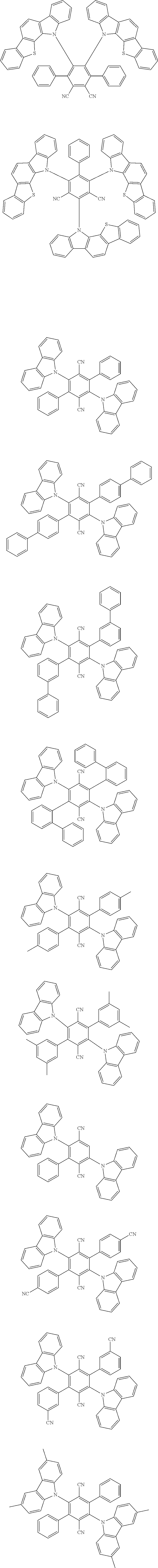

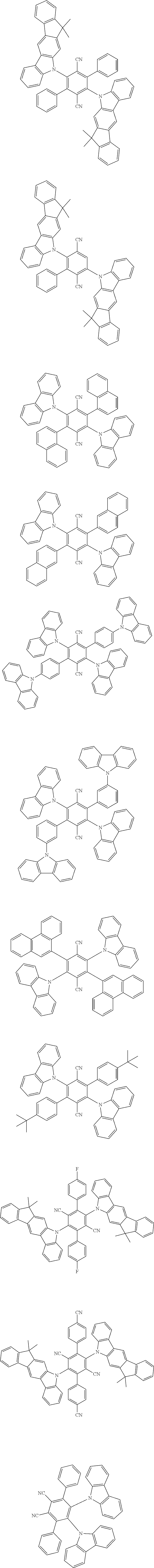

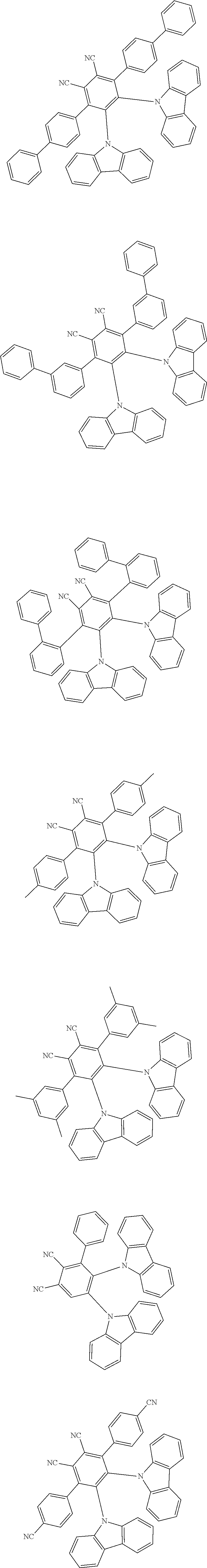

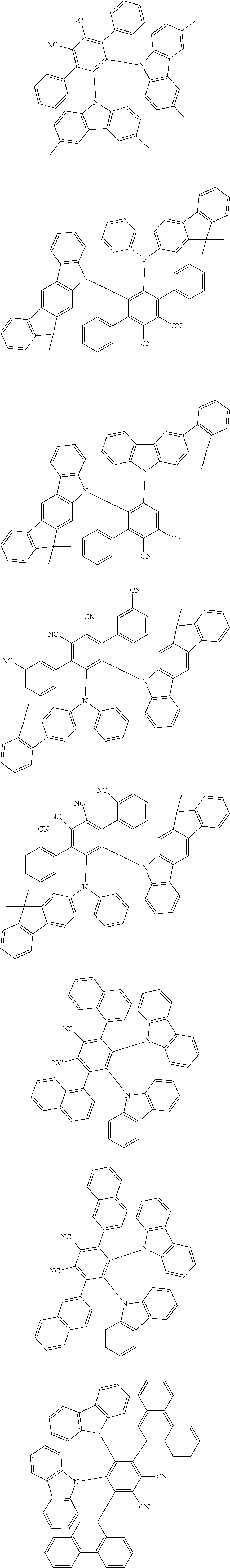

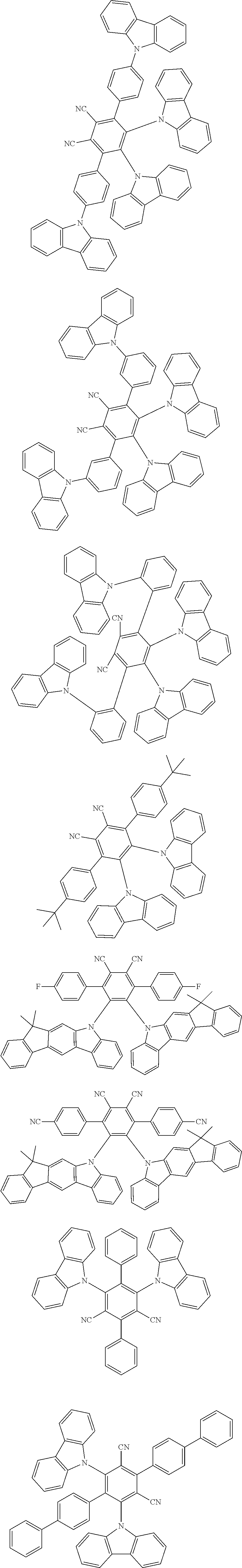

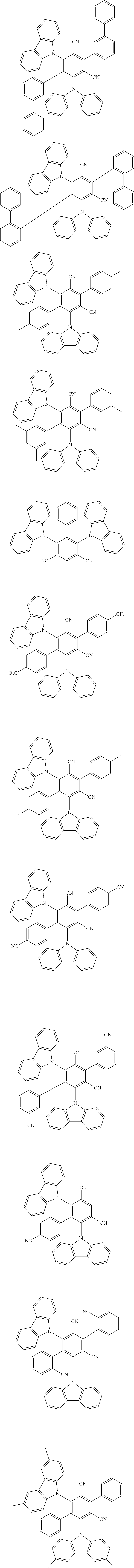

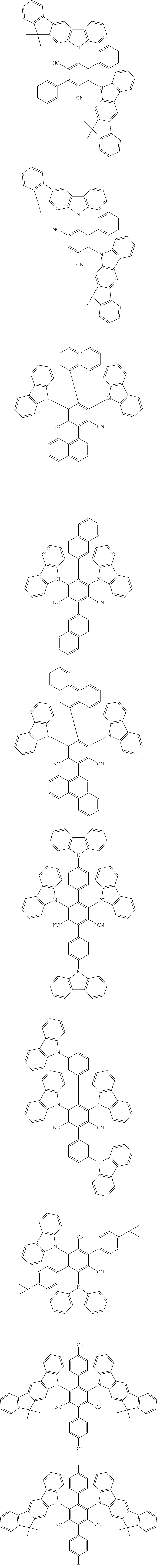

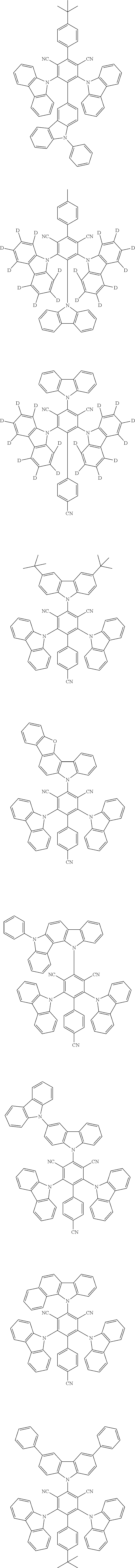

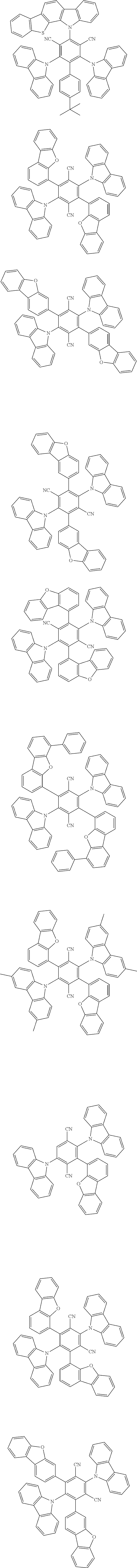

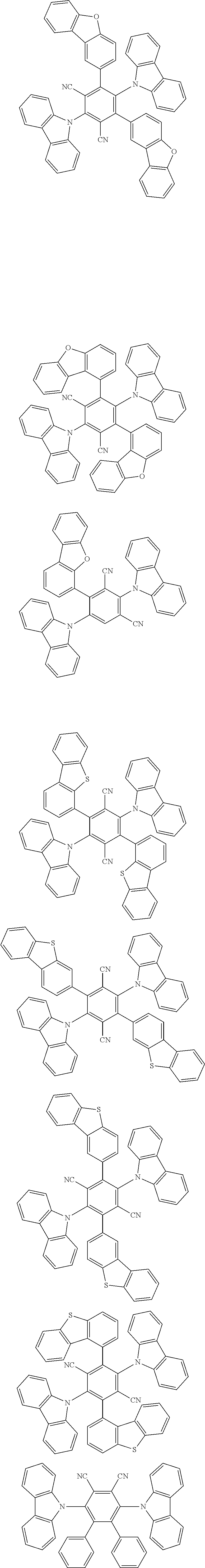

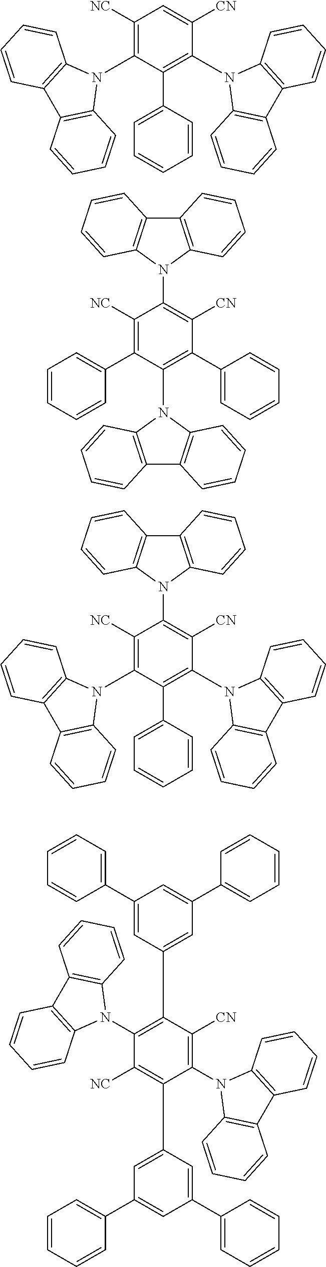

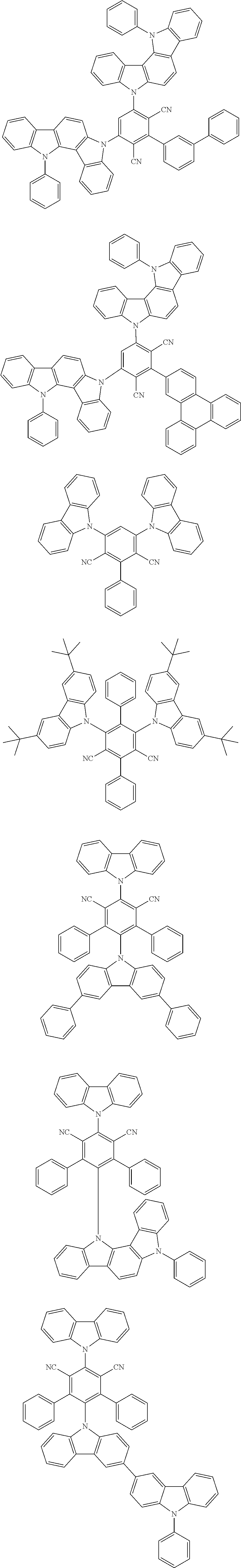

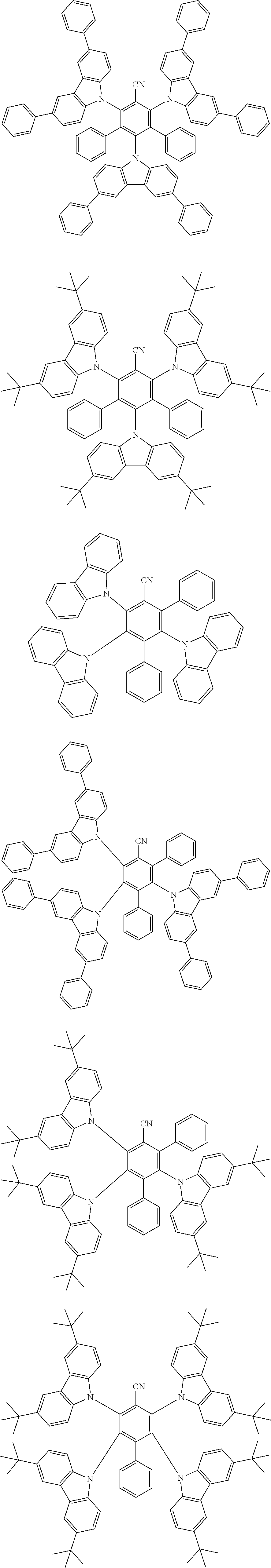

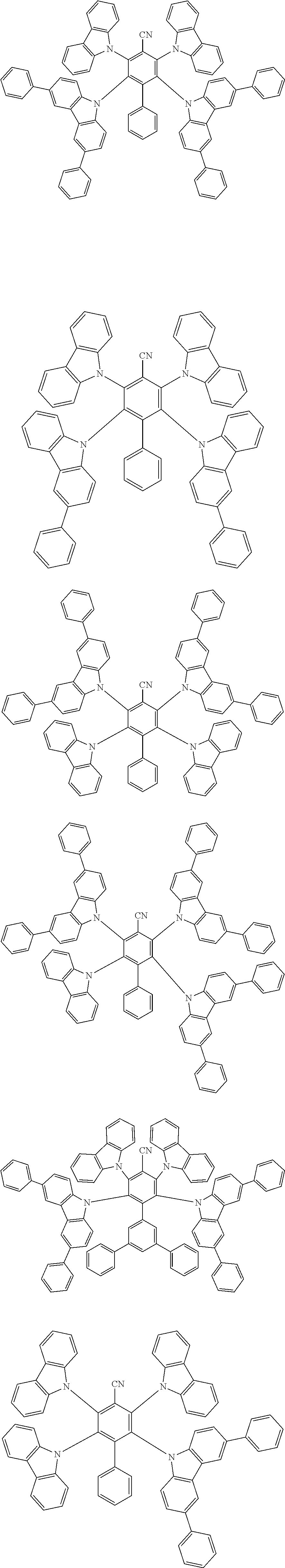

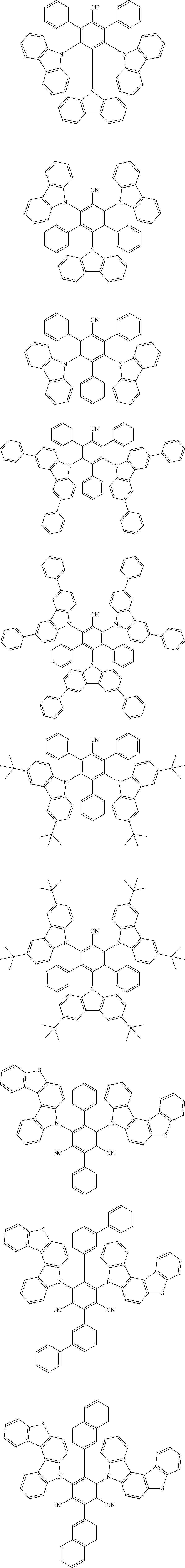

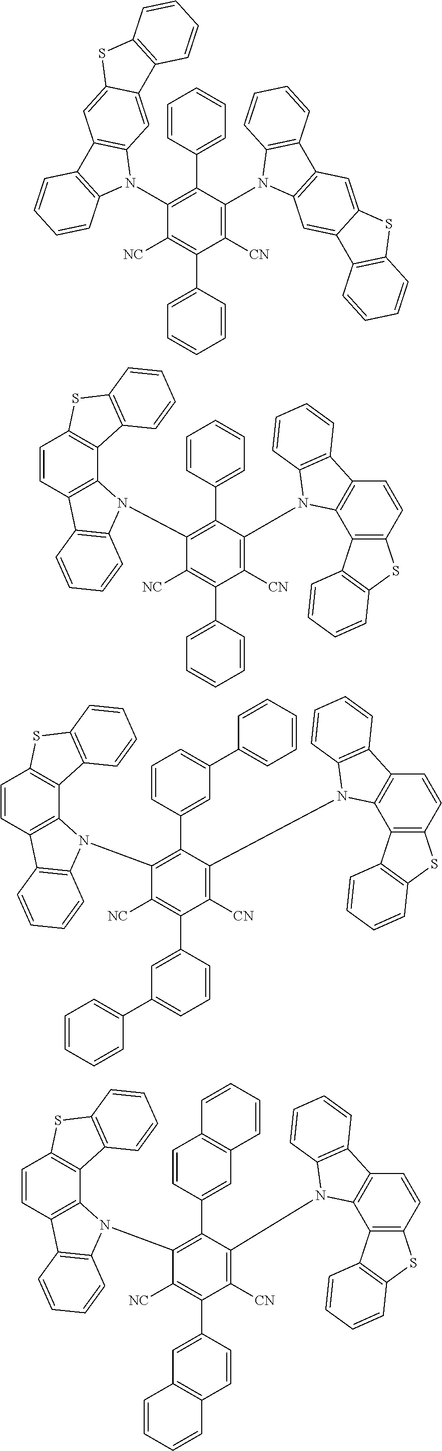

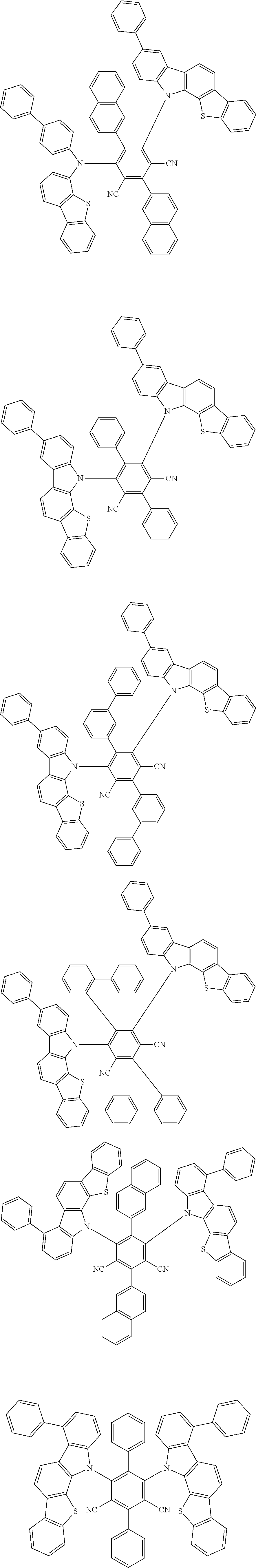

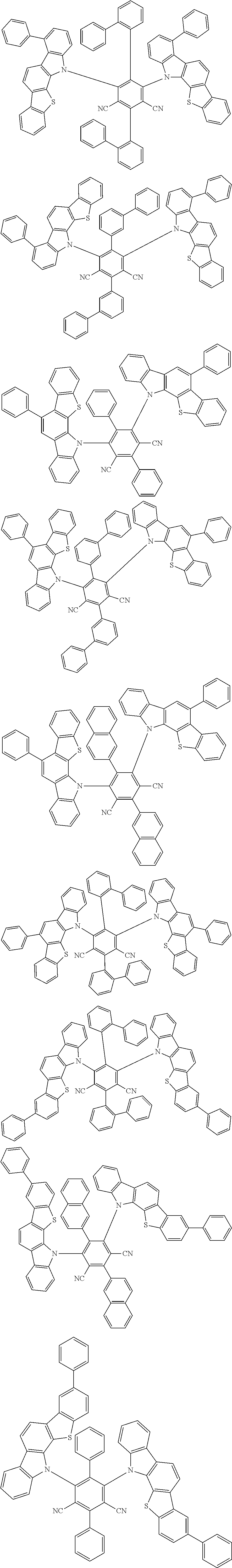

### Compound M3

The emitting layer of the organic EL device in the exemplary embodiment contains a compound M3 represented by a formula (3) below.

The compound M3 in the exemplary embodiment may be a compound exhibiting thermally activated delayed fluorescence or a compound not exhibiting thermally activated delayed fluorescence, however, is preferably a compound not exhibiting thermally activated delayed fluorescence.

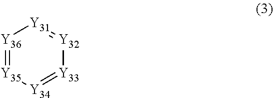

In the formula (3):

Y₃₁ to Y₃₆ are each independently CR₃ or a nitrogen atom;

two or more of Y₃₁ to Y₃₆ are each a nitrogen atom;

when a plurality of R₃ are present, at least one combination of adjacent two or more of the plurality of R₃ are mutually bonded to form a substituted or unsubstituted monocyclic ring, mutually bonded to form a substituted or unsubstituted fused ring, or not mutually bonded; and

R₃ not forming the substituted or unsubstituted monocyclic ring and not forming the substituted or unsubstituted fused ring is each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, a substituted or unsubstituted heterocyclic group having 5 to 50 ring atoms, or a group represented by a formula (3A) below.

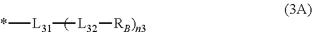

In the formula (3A):

R_{B} is a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms;

when a plurality of R_{B} are present, the plurality of R_{B} are mutually the same or different;

L₃₁ is: a single bond; a substituted or unsubstituted arylene group having 6 to 50 ring carbon atoms, or a trivalent, tetravalent, pentavalent, or hexavalent group derived from the arylene group;

a substituted or unsubstituted divalent heterocyclic group having 5 to 50 ring atoms, or a trivalent, tetravalent, pentavalent, or hexavalent group derived from the heterocyclic group; or

a divalent group formed by bonding two groups selected from the group consisting of a substituted or unsubstituted arylene group having 6 to 50 ring carbon atoms and a substituted or unsubstituted divalent heterocyclic group having 5 to 50 ring atoms, or a trivalent, tetravalent, pentavalent, or hexavalent group derived from the divalent group;

L₃₂ is a single bond, a substituted or unsubstituted arylene group having 6 to 50 ring carbon atoms, or a substituted or unsubstituted divalent heterocyclic group having 5 to 50 ring atoms;

n₃ is 1, 2, 3, 4, or 5;

when L₃₁ is a single bond, n₃ is 1, and L₃₂ is bonded to a carbon atom in a six-membered ring in the formula (3);

when a plurality of L₃₂ are present, the plurality of L₃₂ are mutually the same or different; and

* represents a bonding position to a carbon atom in a six-membered ring in the formula (3).

The compound M3 preferably does not include a pyridine ring in a molecule.

The compound M3 is also preferably a compound represented by a formula (31) or a formula (32) below.

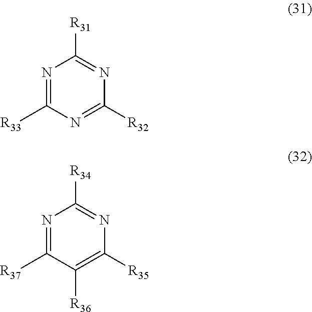

In the formula (32): at least one combination of adjacent two or more of R₃₅ to R₃₇ are mutually bonded to form a substituted or unsubstituted monocyclic ring, mutually bonded to form a substituted or unsubstituted fused ring, or not mutually bonded.

R₃₁ to R₃₃ in the formula (31), R₃₄ in the formula (32), and R₃₅ to R₃₇ not forming the substituted or unsubstituted monocyclic ring and not forming the substituted or unsubstituted fused ring each independently represent the same as R₃ in the formula (3).

The compound M3 is also preferably a compound represented by a formula (31) below.

The organic EL device of the exemplary embodiment may satisfy any one condition of the following conditions (PRV-1) to (PRV-6).

### Condition (PRV-1)

When the compound M3 is a compound Mx3 represented by a formula (301) below and a singlet energy Si(Mx3) of the compound Mx3 is larger than a singlet energy S₁(M2) of the compound M2, the emitting layer does not contain a compound having a larger singlet energy than the singlet energy S1(M2) of the compound M2, other than the compound Mx3.

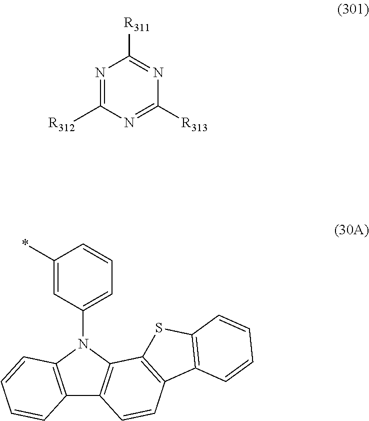

In the formula (301):

R₃₁₁ is a phenyl structure;

R₃₁₂ is a biphenyl structure; and

R₃₁₃ is a structure represented by the formula (30A).

### Condition (PRV-2)

When the compound M3 is a compound Mx32 represented by a formula (302) below and a singlet energy Si(Mx32) of the compound Mx32 is larger than the singlet energy S₁(M2) of the compound M2, the emitting layer does not contain a compound having a larger singlet energy than the singlet energy S₁(M2) of the compound M2, other than the compound Mx32.

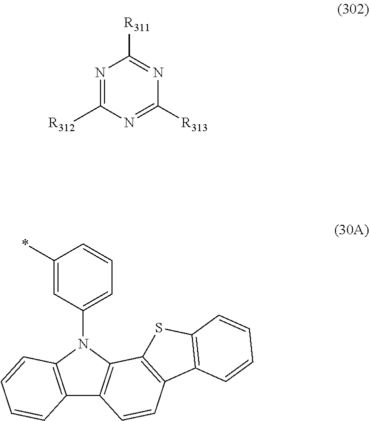

In the formula (302):

R₃₁₁ includes a phenyl structure;

R₃₁₂ includes a biphenyl structure; and

R₃₁₃ includes a structure represented by the formula (30A).

### Condition (PRV-3)

When the compound M3 is a compound Mx33 represented by a formula (303) below and a singlet energy Si(Mx33) of the compound Mx33 is larger than the singlet energy S₁(M2) of the compound M2, the emitting layer does not contain a compound having a larger singlet energy than the singlet energy S₁(M2) of the compound M2, other than the compound Mx33.

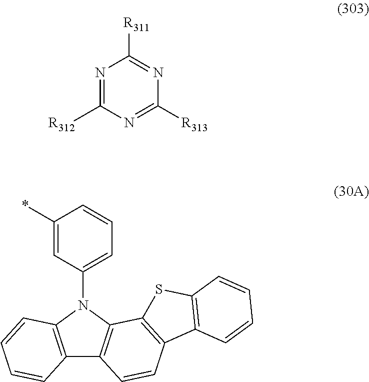

In the formula (303):

one of R₃₁₁, R₃₁₂, and R₃₁₃ is a structure represented by the formula (30A); and

the remaining two of R₃₁₁, R₃₁₂, and R₃₁₃ each independently represent the same as R₃ in the formula (3).

### Condition (PRV-4)

When the compound M3 is a compound Mx34 represented by a formula (31) below and a singlet energy S₁(Mx34) of the compound Mx34 is larger than a singlet energy S₁(M2) of the compound M2, the emitting layer does not contain a compound having a larger singlet energy than the singlet energy S₁(M2) of the compound M2, other than the compound Mx34.

### Condition (PRV-5)

When the compound M3 is a compound Mx35 represented by a formula (30A) below and a singlet energy Si(Mx35) of the compound Mx35 is larger than a singlet energy S₁(M2) of the compound M2, the emitting layer does not contain a compound having a larger singlet energy than the singlet energy S₁(M2) of the compound M2, other than the compound Mx35.

### Condition (PRV-6)

The emitting layer does not contain a compound having a larger singlet energy than the singlet energy S₁(M2) of the compound M2, other than the compound M3.

In the compound M3, it is preferable that L₃₁ is: a single bond; a substituted or unsubstituted arylene group having 6 to 50 ring carbon atoms, or a trivalent, tetravalent, pentavalent, or hexavalent group derived from the arylene group; or a divalent group formed by bonding two groups selected from the group consisting of a substituted or unsubstituted arylene group having 6 to 50 ring carbon atoms, or a trivalent, tetravalent, pentavalent, or hexavalent group derived from the divalent group; and L₃₂ is a single bond, or a substituted or unsubstituted arylene group having 6 to 50 ring carbon atoms.

In the compound M3, it is preferable that L₃₁ is a single bond, or a substituted or unsubstituted arylene group having 6 to 50 ring carbon atoms; n₃ is 1; and L₃₂ is a single bond, or a substituted or unsubstituted arylene group having 6 to 50 ring carbon atoms.

In the compound M3, it is preferable that L₃₁ is a single bond, a substituted or unsubstituted phenylene group, a substituted or unsubstituted biphenylene group, or a divalent group formed by bonding two groups selected from the group consisting of a substituted or unsubstituted phenylene group and a substituted or unsubstituted biphenylene group, or a trivalent, tetravalent, pentavalent, or hexavalent group derived from the divalent group; n₃ is 1; and L₃₂ is a single bond, a substituted or unsubstituted phenylene group, or a substituted or unsubstituted biphenylene group.

R₃ in the formula (3) is each independently preferably a hydrogen atom, a substituted or unsubstituted alkyl group having 1 to 50 carbon atoms, a substituted or unsubstituted aryl group having 6 to 50 ring carbon atoms, a substituted or unsubstituted heterocyclic group having 5 to 50 ring atoms, or a compound represented by the formula (3A).

R₃ in the formula (3) is each independently preferably a hydrogen atom, a substituted or unsubstituted alkyl group having 1 to 50 carbon atoms, a substituted or unsubstituted aryl group having 6 to 50 ring carbon atoms, or a compound represented by the formula (3A).

The compound M3 preferably contains, in a molecule, at least one group selected from the group consisting of groups represented by formulae (A31) to (A44) below.

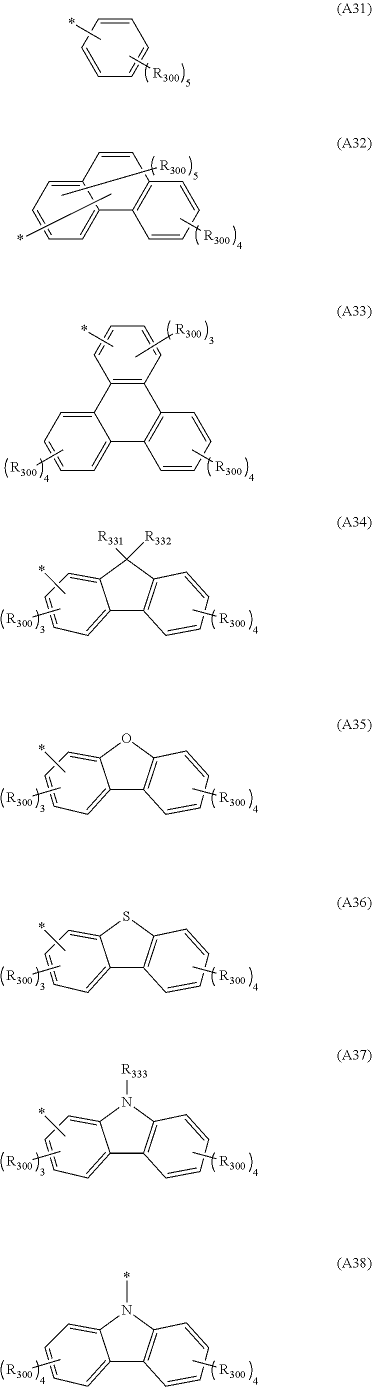

In the formulae (A31) to (A38):

at least one combination of adjacent two or more of a plurality of R₃₀₀ are mutually bonded to form a substituted or unsubstituted monocyclic ring, mutually bonded to form a substituted or unsubstituted fused ring, or not mutually bonded;

a combination of R₃₃₁ and R₃₃₂ are mutually bonded to form a substituted or unsubstituted monocyclic ring, mutually bonded to form a substituted or unsubstituted fused ring, or not mutually bonded;

R₃₀₀, R₃₃₁ and R₃₃₂ not forming the substituted or unsubstituted monocyclic ring and not forming the substituted or unsubstituted fused ring, and R₃₃₃ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms; and

* in each of the formulae (A31) to (A38) represents a bonding position to any other atom in a molecule of the compound M3.

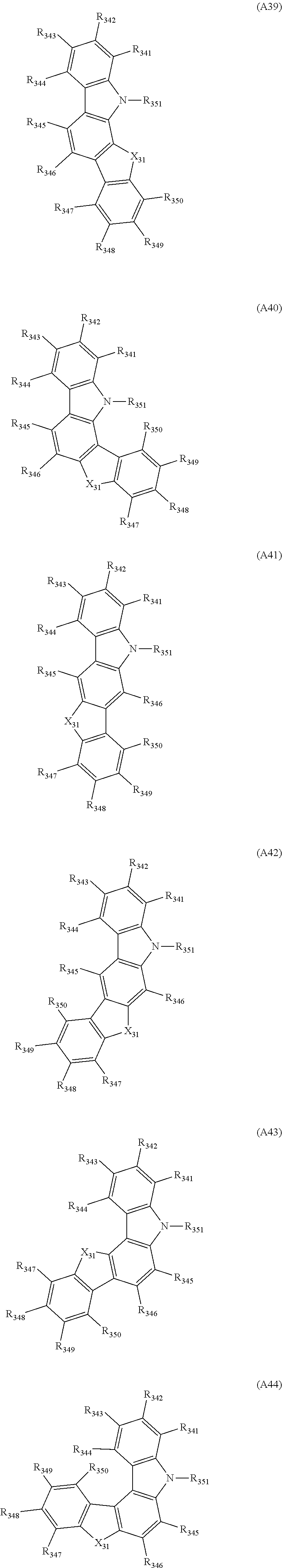

In the formulae (A39) to (A44):

at least one combination of adjacent two or more of R₃₄₁ to R₃₅₀ are mutually bonded to form a substituted or unsubstituted monocyclic ring, mutually bonded to form a substituted or unsubstituted fused ring, or not mutually bonded;

at least one of R₃₄₁ to R₃₅₁ represents a bonding position to any other atom in a molecule of the compound M3;

X₃₁ is a sulfur atom, an oxygen atom, NR₃₅₂, or CR₃₅₃R₃₅₄;

a combination of R₃₅₃ and R₃₅₄ are mutually bonded to form a substituted or unsubstituted monocyclic ring, mutually bonded to form a substituted or unsubstituted fused ring, or not mutually bonded; and

R₃₄₁ to R₃₅₁ not being a bonding position to any other atom in a molecule of the compound M3, not forming the substituted or unsubstituted monocyclic ring, and not forming the substituted or unsubstituted fused ring; R₃₅₂; and R₃₅₃ and R₃₅₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms.

The compound M3 preferably contains, in a molecule, at least one group selected from the group consisting of groups represented by the formulae (A38) to (A44).

In the compound M3, it is preferable that at least one of Y₃₁ to Y₃₆ is CR₃, at least one R₃ is a group represented by the formula (3A), and R_{B} is any one of groups represented by the formulae (A31) to (A44).

In the compound M3, it is preferable that at least one of Y₃₁ to Y₃₆ is CR₃, at least one R₃ is a group represented by the formula (3A), and R_{B} is any one of groups represented by the formulae (A38) to (A44).

In the compound M3, it is preferable that R₃₅₂ is a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M3, it is preferable that a combination of R₃₅₃ and R₃₅₄ are mutually bonded to form a substituted or unsubstituted monocyclic ring, mutually bonded to form a substituted or unsubstituted fused ring, or not mutually bonded; and R₃₅₃ and R₃₅₄ not forming the substituted or unsubstituted monocyclic ring and not forming the substituted or unsubstituted fused ring are each independently a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

In the compound M3, it is preferable that a substituent for the “substituted or unsubstituted” group is an unsubstituted alkyl group having 1 to 25 carbon atoms, an unsubstituted alkenyl group having 2 to 25 carbon atoms, an unsubstituted alkynyl group having 2 to 25 carbon atoms, an unsubstituted cycloalkyl group having 3 to 25 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), an unsubstituted aralkyl group having 7 to 50 carbon atoms, a group represented by —C(═O)R₉₀₈, a group represented by —COOR₉₀₉, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a group represented by —S(═O)₂R₉₃₈, a halogen atom, a cyano group, a nitro group, an unsubstituted aryl group having 6 to 25 ring carbon atoms, or an unsubstituted heterocyclic group having 5 to 25 ring atoms; and R₉₀₁ to R₉₀₉ and R₉₃₁ to R₉₃₈ are each independently a hydrogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compound M3, it is preferable that a substituent for the “substituted or unsubstituted” group is a halogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compound M3, it is preferable that a substituent for the “substituted or unsubstituted” group is an unsubstituted alkyl group having 1 to 10 carbon atoms, an unsubstituted aryl group having 6 to 12 ring carbon atoms, or an unsubstituted heterocyclic group having 5 to 12 ring atoms.

In the compound M3, it is also preferable that all of the “substituted or unsubstituted” groups are “unsubstituted” groups.

### Manufacturing Method of Compound M3

The compound M3 according to the exemplary embodiment can be manufactured by a known method.

### Specific Examples of Compound M3

Specific examples of the compound M3 of the exemplary embodiment include compounds below. It should however be noted that the invention is not limited to the specific examples of the compound.

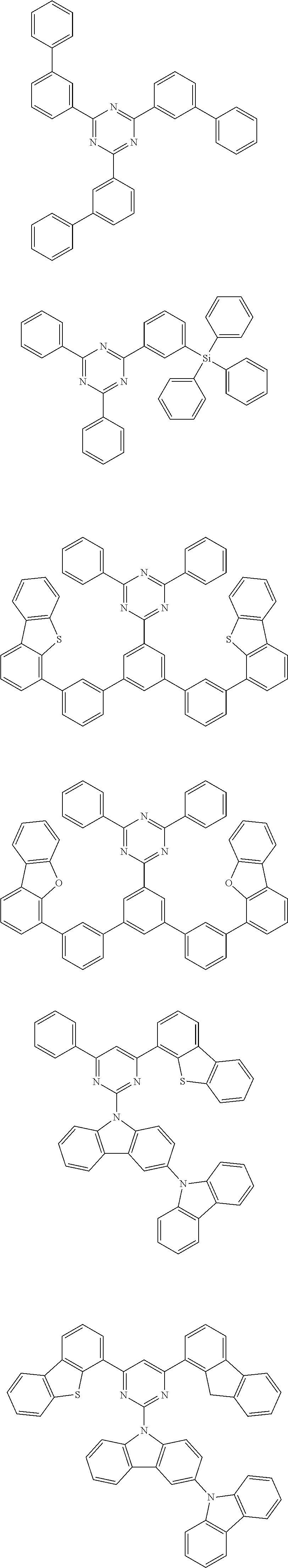

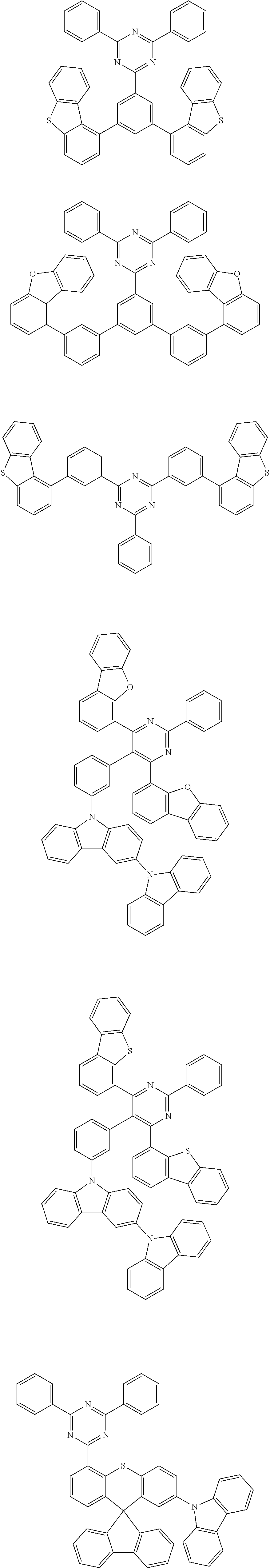

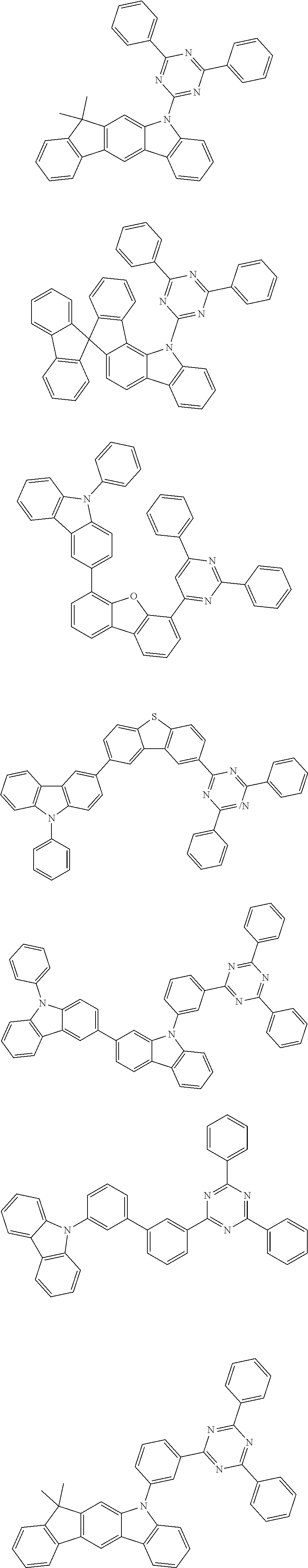

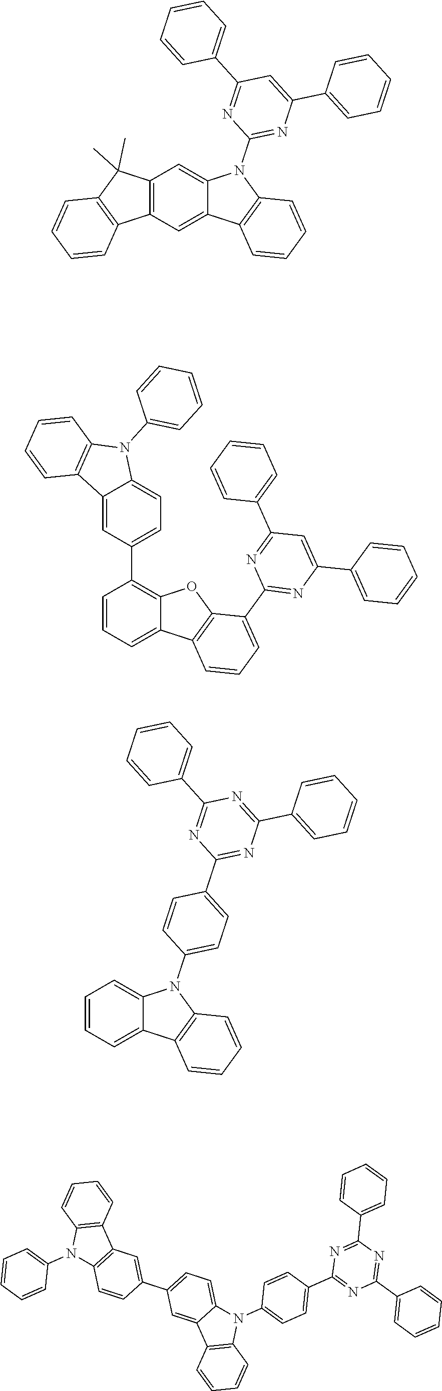 [SMILES: CC1(C)c2ccccc2-c2cc3c4ccccc4n(-c4nc(-c5ccccc5)cc(-c5ccccc5)n4)c3cc21 ; c1ccc(-c2cc(-c3ccccc3)nc(-c3cccc4c3oc3c(-c5ccc6c(c5)c5ccccc5n6-c5ccccc5)cccc34)n2)cc1 ; c1ccc(-c2nc(-c3ccccc3)nc(-c3ccc(-n4c5ccccc5c5ccccc54)cc3)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

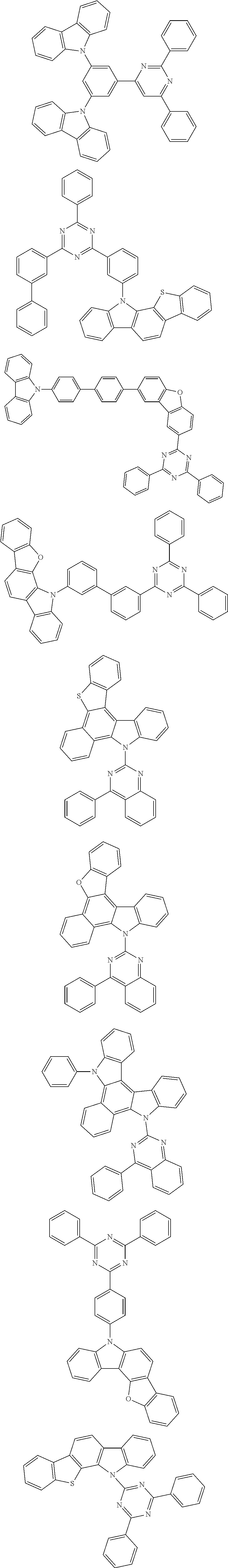

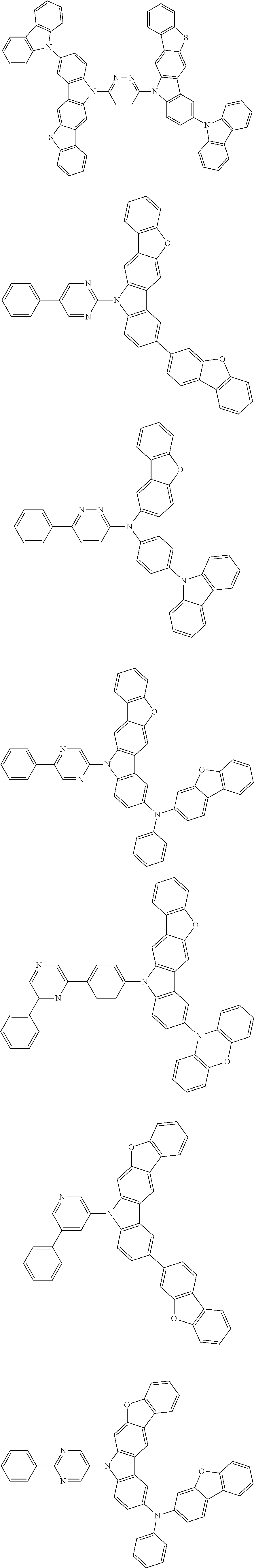

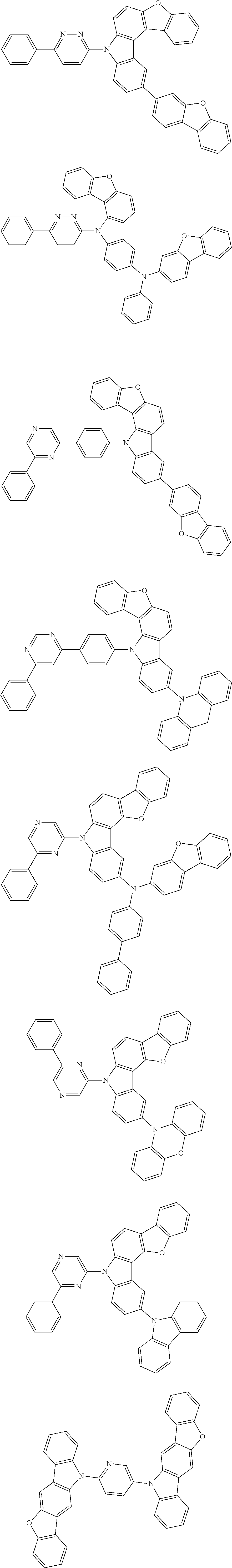

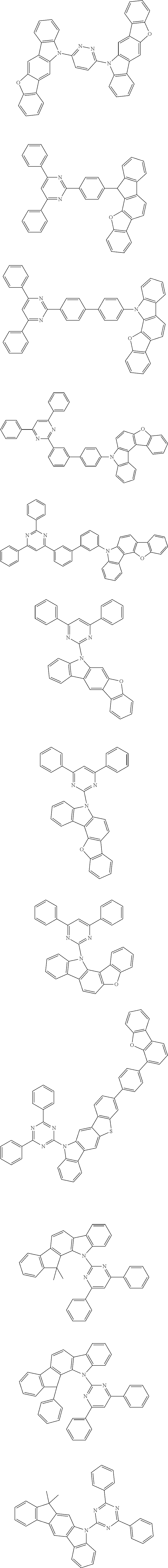

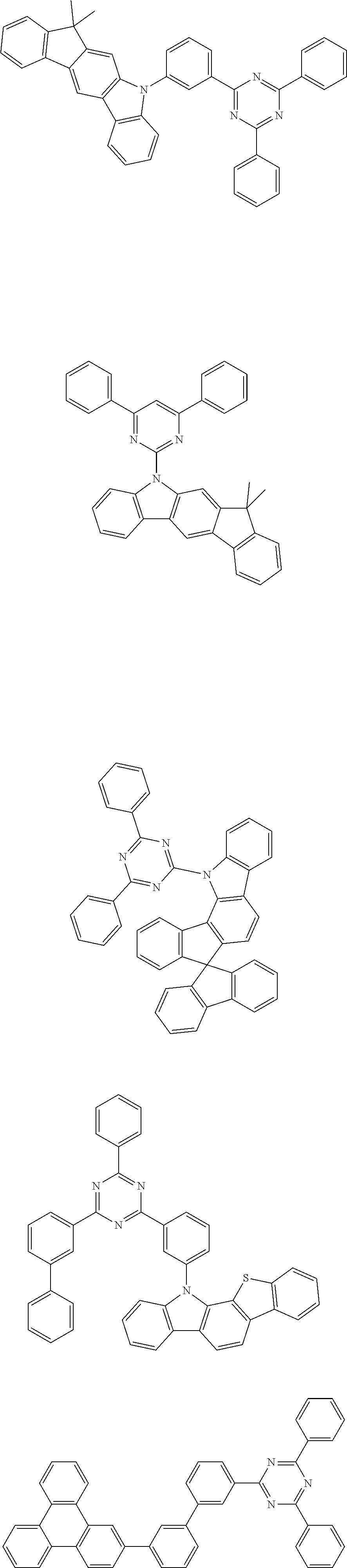 [SMILES: CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)cc(-c5ccccc5)n4)c3cc21 ; c1ccc(-c2nc(-c3ccccc3)nc(-n3c4ccccc4c4ccc5c(c43)-c3ccccc3C53c4ccccc4-c4ccccc43)n2)cc1 ; c1ccc(-c2cccc(-c3nc(-c4ccccc4)nc(-c4cccc(-n5c6ccccc6c6ccc7c8ccccc8sc7c65)c4)n3)c2)cc1 ; c1ccc(-c2nc(-c3ccccc3)nc(-c3cccc(-c4cccc(-c5ccc6c7ccccc7c7ccccc7c6c5)c4)c3)n2)cc1]

Relationship between Compound M2 and Compound M3 in Emitting Layer

In the organic EL device of the exemplary embodiment, the lowest singlet energy S₁(M2) of the compound M2 and the lowest singlet energy S₁(M3) of the compound M3 satisfy a relationship of the numerical formula (Numerical Formula 1).

An energy gap T_{77K}(M3) at 77K of the compound M3 is preferably larger than an energy gap T_{77K}(M2) at 77K of the compound M2. In other words, a relationship of the following numerical formula (Numerical Formula 5) is preferably satisfied.

T_{77K}(M3)>T_{77K}(M2) (Numerical Formula 5)

It is preferable that mainly the compound M2 emits light in the emitting layer when the organic EL device of the exemplary embodiment emits light.

### TADF Mechanism

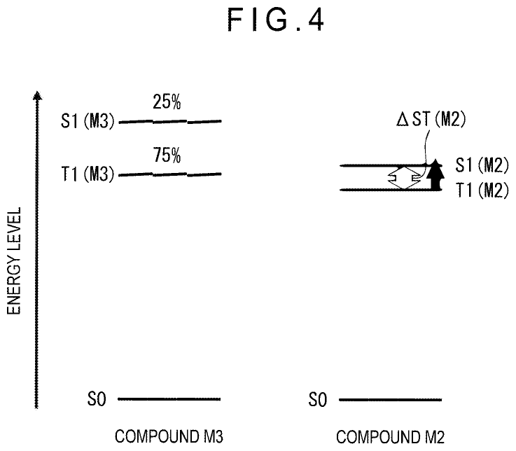

FIG. 4 shows an example of a relationship between energy levels of the compound M3 and the compound M2 in the emitting layer. In FIG. 4, S0 represents a ground state. S1(M2) represents the lowest singlet state of the compound M2. T1(M2) represents the lowest triplet state of the compound M2. S1(M3) represents the lowest singlet state of the compound M3. T1(M3) represents the lowest triplet state of the compound M3. As shown in FIG. 4, when a material having a small ΔST(M2) is used as the compound M2, inverse intersystem crossing can be caused by a heat energy from the lowest triplet state T1(M2) to the lowest singlet state S1(M2) in the compound M2.

The inverse intersystem crossing caused in the compound M2 enables light emission from the lowest singlet state S1(M2) of the compound M2 can be observed when the emitting layer does not contain a fluorescent dopant with the lowest singlet state S1 smaller than the lowest singlet state S1(M2) of the compound M2. It is inferred that the internal quantum efficiency can be theoretically raised up to 100% also by using delayed fluorescence by the TADF mechanism.

### Film Thickness of Emitting Layer

A film thickness of the emitting layer of the organic EL device in the exemplary embodiment is preferably in a range of 5 nm to 50 nm, more preferably in a range of 7 nm to 50 nm, further preferably in a range of 10 nm to 50 nm. When the film thickness of the emitting layer is 5 nm or more, the formation of the emitting layer and the adjustment of the chromaticity are easy. When the film thickness of the emitting layer is 50 nm or less, an increase in the drive voltage is likely to be reducible.

### Content Ratios of Compounds in Emitting Layer

Content ratios of the compounds M2 and M3 in the emitting layer preferably fall, for instance, within a range below.

The content ratio of the compound M2 is preferably in a range from 10 mass % to 80 mass %, more preferably in a range from 10 mass % to 60 mass %, further preferably in a range from 20 mass % to 60 mass %.

The content ratio of the compound M3 is preferably in a range from 20 mass % to 90 mass %, more preferably in a range from 40 mass % to 90 mass %, further preferably in a range from 40 mass % to 80 mass %.

It should be noted that the emitting layer of the exemplary embodiment may further contain material(s) other than the compounds M2 and M3.

The emitting layer may include a single type of the compound M2 or may include two or more types of the compound M2. The emitting layer may include a single type of the compound M3 or may include two or more types of the compound M3.

An arrangement of an organic EL device will be further described below.

### Substrate

The substrate is used as a support for the organic EL device. For instance, glass, quartz, plastics and the like are usable for the substrate. A flexible substrate is also usable. The flexible substrate means a substrate that can be bent. Examples of the flexible substrate include a plastic substrate made using polycarbonate, polyarylate, polyethersulfone, polypropylene, polyester, polyvinyl fluoride, and polyvinyl chloride. Moreover, an inorganic vapor deposition film is also usable.

### Anode

Metal having a large work function (specifically, 4.0 eV or more), an alloy, an electrically conductive compound and a mixture thereof are preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, and a mixture thereof, which have a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide are usable for forming the cathode regardless of a magnitude of the work function.

The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(Vs) or more.

For the hole transporting layer, a carbazole derivative such as CBP, CzPA, and PCzPA and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. A layer containing the substance exhibiting a higher hole transportability may be provided in the form of a single layer or a laminated layer of two or more layers of the above substance(s).

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. The above-described substances mostly have an electron mobility of 10⁻⁶ cm²/(Vs) or more. It should be noted that any substance other than the above substance may be used for the electron transporting layer as long as the substance exhibits a higher electron transportability than the hole transportability. Moreover, the electron transporting layer may be provided in the form of a single layer or a laminated layer of two or more layers of the above substance(s).

Further, a high polymer compound is usable for the electron transporting layer. For instance, poly[(9,9-dihexylfluorene-2,7-diyl)-co-(pyridine-3,5-diyl)] (abbreviation: PF-Py), poly[(9,9-dioctylfluorene-2,7-diyl)-co-(2,2′-bipyridine-6,6′-diyl)] (abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic, layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

According to the exemplary embodiment, an organic EL device having a high performance, especially high luminous efficiency, can be provided. The organic EL device according to the exemplary embodiment is applicable to an electronic device such as a display device and a light-emitting device.

### Second Exemplary Embodiment

An arrangement of an organic EL device according to a second exemplary embodiment will be described below. In the description of the second exemplary embodiment, the same components as those in the first exemplary embodiment are denoted by the same reference signs and names to simplify or omit an explanation of the components. In the second exemplary embodiment, any materials and compounds that are not specified may be the same as those in the first exemplary embodiment.

The organic EL device according to the second exemplary embodiment is different from the organic EL device according to the first exemplary embodiment in that the emitting layer further includes a fluorescent compound M1. The second exemplary embodiment is the same as the first exemplary embodiment in other respects.

Specifically, in the second exemplary embodiment, the emitting layer contains the compound M3 represented by the formula (3), the delayed fluorescent compound M2 represented by the formula (2), and the fluorescent compound M1. The compound M1 is preferably a compound not exhibiting thermally activated delayed fluorescence.

In this arrangement, the compound M1 is preferably a dopant material, the compound M2 is preferably a host material, and the compound M3 is preferably not a dopant material.

### Compound M1

The compound M1 of the exemplary embodiment is not a phosphorescent metal complex. The compound M1 is preferably not a heavy metal complex. The compound M1 is preferably not a metal complex.

A fluorescent material is usable as the compound M1 of the exemplary embodiment. Specific examples of the fluorescent material include a bisarylaminonaphthalene derivative, aryl-substituted naphthalene derivative, bisarylaminoanthracene derivative, aryl-substituted anthracene derivative, bisarylaminopyrene derivative, aryl-substituted pyrene derivative, bisarylamino chrysene derivative, aryl-substituted chrysene derivative, bisarylaminofluoranthene derivative, aryl-substituted fluoranthene derivative, indenoperylene derivative, acenaphthofluoranthene derivative, compound including a boron atom, pyromethene boron complex compound, compound having a pyromethene skeleton, metal complex of the compound having a pyrromethene skeleton, diketopyrrolopyrrole derivative, perylene derivative, and naphthacene derivative.

The compound M1 is preferably a compound that emits light having the maximum peak wavelength in a range from 400 nm to 700 nm.

Herein, the maximum peak wavelength means a peak wavelength of a fluorescence spectrum exhibiting a maximum luminous intensity among fluorescence spectra measured in a toluene solution in which a measurement target compound is dissolved at a concentration ranging from 10⁻⁶ mol/1 to 10⁻⁵ mol/l. A spectrophotofluorometer (manufactured by Hitachi High-Tech Science Corporation: F-7000) is used as a measuring device.

The compound M1 preferably exhibits red or green light emission.

Herein, the red light emission refers to light emission whose maximum peak wavelength of fluorescence spectrum is in a range from 600 nm to 660 nm.

When the compound M1 is a red fluorescent compound, the maximum peak wavelength of the compound M1 is preferably in a range from 600 nm to 660 nm, more preferably in a range from 600 nm to 640 nm, further preferably in a range from 610 nm to 630 nm.

Herein, the green light emission refers to light emission whose maximum peak wavelength of fluorescence spectrum is in a range from 500 nm to 560 nm.

When the compound M1 is a green fluorescent compound, the maximum peak wavelength of the compound M1 is preferably in a range from 500 nm to 560 nm, more preferably in a range from 500 nm to 540 nm, further preferably in a range from 510 nm to 540 nm.

Herein, the blue light emission refers to light emission whose maximum peak wavelength of fluorescence spectrum is in a range from 430 nm to 480 nm.

When the compound M1 is a blue fluorescent compound, the maximum peak wavelength of the compound M1 is preferably in a range from 430 nm to 480 nm, more preferably in a range from 440 nm to 480 nm.

The maximum peak wavelength of light emitted from the organic EL device is measured as follows.

Voltage is applied on the organic EL devices such that a current density becomes 10 mA/cm², where spectral radiance spectrum is measured by a spectroradiometer CS-2000 (manufactured by Konica Minolta, Inc.).

A peak wavelength of an emission spectrum, a luminous intensity of which is the maximum in the obtained spectral radiance spectrum, is measured and defined as the maximum peak wavelength (unit: nm).

The compound M1 is also preferably a compound represented by a formula (1) below.

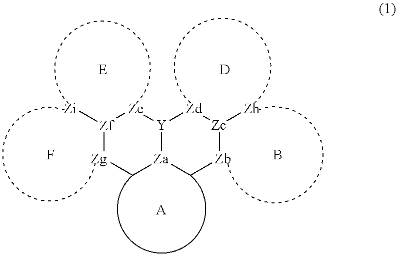

In the formula (1):

a ring A, ring B, ring D, ring E, and ring F are each independently a cyclic structure selected from the group consisting of a substituted or unsubstituted aryl ring having 6 to 30 ring carbon atoms and a substituted or unsubstituted heterocycle having 5 to 30 ring atoms;

one of the ring B and the ring D is present or both of the ring B and the ring D are present;

when both of the ring B and the ring D are present, the ring B and the ring D share a bond between Zc and Zh;

one of the ring E and the ring F is present or both of the ring E and the ring F are present;

when both of the ring E and the ring F are present, the ring E and the ring F share a bond between Zf and Zi;

Za is a nitrogen atom or a carbon atom;

Zb is a nitrogen atom or a carbon atom when the ring B is present;

Zb is an oxygen atom, a sulfur atom, NRb, C(Rb₁)(Rb₂), or Si(Rb₃)(Rb₄) when the ring B is not present;

Zc is a nitrogen atom or a carbon atom;

Zd is a nitrogen atom or a carbon atom when the ring D is present; Zd is an oxygen atom, a sulfur atom, or NRd when the ring D is not present;

Ze is a nitrogen atom or a carbon atom when the ring E is present;

Ze is an oxygen atom, a sulfur atom, or NRe when the ring E is not present;

Zf is a nitrogen atom or a carbon atom;

Zg is a nitrogen atom or a carbon atom when the ring F is present;

Zg is an oxygen atom, a sulfur atom, NRg, C(Rg₁)(Rg₂), or Si(Rg₃)(Rg₄) when the ring F is not present;

Zh is a nitrogen atom or a carbon atom;

Zi is a nitrogen atom or a carbon atom;

Y is a boron atom, a phosphorus atom, SiRh, P═O or P═S,

Rb, Rb₁, Rb₂, Rb₃, Rb₄, Rd, Re, Rg, Rg₁, Rg₂, Rg₃, Rg₄, and Rh are each independently a hydrogen atom or a substituent;

Rb, Rb₁, Rb₂, Rb₃, Rb₄, Rd, Re, Rg, Rg₁, Rg₂, Rg₃, Rg₄, and Rh as a substituent are each independently a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted cycloalkyl group having 3 to 30 ring carbon atoms, a group represented by —Si(R₉₁₁)(R₉₁₂)(R₉₁₃), a group represented by —O—(R₉₁₄), a group represented by —S—(R₉₁₅), or a group represented by —N(R₉₁₆)(R₉₁₇);

a bond between Y and Za, a bond between Y and Zd, and a bond between Y and Ze are each a single bond; and

a bond between Y and Za, a bond between Y and Zd, and a bond between Y and Ze are each a single bond, where the single bond is not a coordinate bond but a covalent bond.

Herein, examples of a heterocycle include cyclic structures (heterocycles) excluding a bond from the examples of a “heterocyclic group” listed in the subtitle “Substituents Mentioned Herein.” These heterocycles may be substituted or unsubstituted.

Herein, examples of an aryl ring include cyclic structures (aryl rings) excluding a bond from the examples of an “aryl group” listed in the subtitle “Substituents Mentioned Herein.” These aryl rings may be substituted or unsubstituted.

In the exemplary embodiment, the compound M1 is also preferably a compound represented by a formula (11) below.

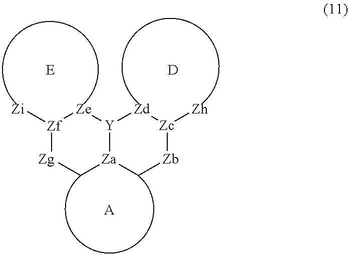

In the formula (11):

a ring A, ring D, and ring E are each independently a cyclic structure selected from the group consisting of a substituted or unsubstituted aryl ring having 6 to 30 ring carbon atoms and a substituted or unsubstituted heterocycle having 5 to 30 ring atoms;

Za is a nitrogen atom or a carbon atom;

Zb is an oxygen atom, a sulfur atom, NRb, C(Rb₁)(Rb₂), or Si(Rb₃)(Rb₄);

Zc is a nitrogen atom or a carbon atom;

Zd is a nitrogen atom or a carbon atom;

Ze is a nitrogen atom or a carbon atom;

Zf is a nitrogen atom or a carbon atom;

Zg is an oxygen atom, a sulfur atom, NRg, C(Rg₁)(Rg₂), or Si(Rg₃)(Rg₄);

Zh is a nitrogen atom or a carbon atom;

Zi is a nitrogen atom or a carbon atom;

Y is a boron atom, a phosphorus atom, SiRh, P═O or P═S, and

Rb, Rb₁, Rb₂, Rb₃, Rb₄, Rg, Rg₁, Rg₂, Rg₃, Rg₄, and Rh each independently represent the same as Rb, Rb₁, Rb₂, Rb₃, Rb₄, Rg, Rg₁, Rg₂, Rg₃, Rg₄, and Rh in the formula (1).

In the exemplary embodiment, the compound M1 is also preferably a compound represented by a formula (15) below.

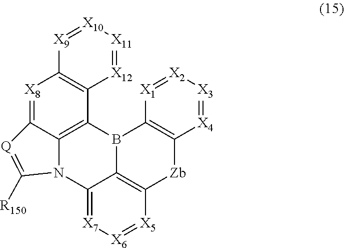

In the formula (15):

Zb is an oxygen atom, a sulfur atom, NRb, C(Rb₁)(Rb₂), or Si(Rb₃)(Rb₄);

X₁ is CR₁₂₁, a nitrogen atom, or a carbon atom bonded to X₁₂ with a single bond;

X₂ is CR₁₂₂ or a nitrogen atom;

X₃ is CR₁₂₃ or a nitrogen atom;

X₄ is CR₁₂₄ or a nitrogen atom;

X₅ is CR₁₂₅ or a nitrogen atom;

X₆ is CR₁₂₆ or a nitrogen atom;

X₇ is CR₁₂₇ or a nitrogen atom;

X₈ is CR₁₂₈ or a nitrogen atom;

X₉ is CR₁₂₉ or a nitrogen atom;

X₁₀ is CR₁₃₀ or a nitrogen atom;

X₁₁ is CR₁₃₁ or a nitrogen atom;

X₁₂ is CR₁₃₂, a nitrogen atom, or a carbon atom bonded to X₁ with a single bond;

Q is CR_{Q} or a nitrogen atom;

at least one combination of adjacent two or more of R₁₂₂ to R₁₃₁ are mutually bonded to form a substituted or unsubstituted monocyclic ring, mutually bonded to form a substituted or unsubstituted fused ring, or not mutually bonded;

at least one combination of adjacent two or more of R₁₂₄, R₁₂₅, Rb, Rb₁, Rb₂, Rb₃ and Rb₄ are mutually bonded to form a substituted or unsubstituted monocyclic ring, mutually bonded to form a substituted or unsubstituted fused ring, or not mutually bonded;

at least one hydrogen atom in a monocyclic ring or a fused ring formed by mutually bonding at least one combination of adjacent two or more of R₁₂₄, R₁₂₅, Rb, Rb₁, Rb₂, Rb₃ and Rb₄ is unsubstituted or substituted by at least one substituent selected from the group consisting of an alkyl group having 1 to 50 carbon atoms, an aryl group having 6 to 50 ring carbon atoms, a heterocyclic group having 5 to 50 ring atoms, a group represented by —O—(R₁₅₁), and a group represented by —N(R₁₅₂)(R₁₅₃); at least one hydrogen atom in the substituent is unsubstituted or substituted by an aryl group having 6 to 50 ring carbon atoms or an alkyl group having 1 to 50 carbon atoms;

R₁₂₁ to R₁₃₂, R₁₅₀, and R_{Q} not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted cycloalkyl group having 3 to 50 ring carbon atoms, a group represented by —Si(R₉₅₁)(R₉₅₂)(R₉₅₃), a group represented by —O—(R₉₅₄), a group represented by —S—(R₉₅₅), a group represented by —N(R₉₅₆)(R₉₅₇), a substituted or unsubstituted aralkyl group having 7 to 50 carbon atoms, a group represented by —C(═O)R₉₅₈, a group represented by —COOR₉₅₉, a halogen atom, a cyano group, a nitro group, a substituted or unsubstituted aryl group having 6 to 50 ring carbon atoms, or a substituted or unsubstituted heterocyclic group having 5 to 50 ring atoms;

Rb, Rb₁, Rb₂, Rb₃, and Rb₄ not forming the substituted or unsubstituted monocyclic ring and not forming the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

R₁₅₁ to R₁₅₃ and R₉₅₁ to R₉₅₉ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

when a plurality of R₁₅₁ are present, the plurality of R₁₅₁ are mutually the same or different;

when a plurality of R₁₅₂ are present, the plurality of R₁₅₂ are mutually the same or different;

when a plurality of R₁₅₃ are present, the plurality of R₁₅₃ are mutually the same or different;

when a plurality of R₉₅₁ are present, the plurality of R₉₅₁ are mutually the same or different;

when a plurality of R₉₅₂ are present, the plurality of R₉₅₂ are mutually the same or different;

when a plurality of R₉₅₃ are present, the plurality of R₉₅₃ are mutually the same or different;

when a plurality of R₉₅₄ are present, the plurality of R₉₅₄ are mutually the same or different;

when a plurality of R₉₅₅ are present, the plurality of R₉₅₅ are mutually the same or different;

when a plurality of R₉₅₆ are present, the plurality of R₉₅₆ are mutually the same or different;

when a plurality of R₉₅₇ are present, the plurality of R₉₅₇ are mutually the same or different;

when a plurality of R₉₅₈ are present, the plurality of R₉₅₈ are mutually the same or different; and

when a plurality of R₉₅₉ are present, the plurality of R₉₅₉ are mutually the same or different.

In the exemplary embodiment, the compound M1 is also preferably a compound represented by a formula (16) below.

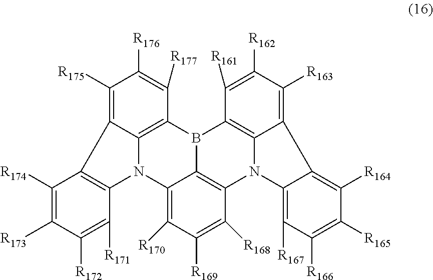

In the formula (16):

at least one combination of adjacent two or more of R₁₆₁ to R₁₇₇ are mutually bonded to form a substituted or unsubstituted monocyclic ring, mutually bonded to form a substituted or unsubstituted fused ring, or not mutually bonded;

R₁₆₁ to R₁₇₇ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₆₁)(R₉₆₂)(R₉₆₃), a group represented by —O—(R₉₆₄), a group represented by —S—(R₉₆₅), a group represented by —N(R₉₆₆)(R₉₆₇), a group represented by —C(═O)R₉₆₈, a group represented by —COOR₉₆₉, a halogen atom, a cyano group, a nitro group, a substituted or unsubstituted aryl group having 6 to 50 ring carbon atoms, or a substituted or unsubstituted heterocyclic group having 5 to 50 ring atoms;

R₉₆₁ to R₉₆₉ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;

when a plurality of R₉₆₁ are present, the plurality of R₉₆₁ are mutually the same or different;

when a plurality of R₉₆₂ are present, the plurality of R₉₆₂ are mutually the same or different;

when a plurality of R₉₆₃ are present, the plurality of R₉₆₃ are mutually the same or different;

when a plurality of R₉₆₄ are present, the plurality of R₉₆₄ are mutually the same or different;

when a plurality of R₉₆₅ are present, the plurality of R₉₆₅ are mutually the same or different;

when a plurality of R₉₆₆ are present, the plurality of R₉₆₆ are mutually the same or different;

when a plurality of R₉₆₇ are present, the plurality of R₉₆₇ are mutually the same or different;

when a plurality of R₉₆₈ are present, the plurality of R₉₆₈ are mutually the same or different; and

when a plurality of R₉₆₉ are present, the plurality of R₉₆₉ are mutually the same or different.

In the exemplary embodiment, the compound M1 is also preferably a compound represented by a formula (171) or (172) below.

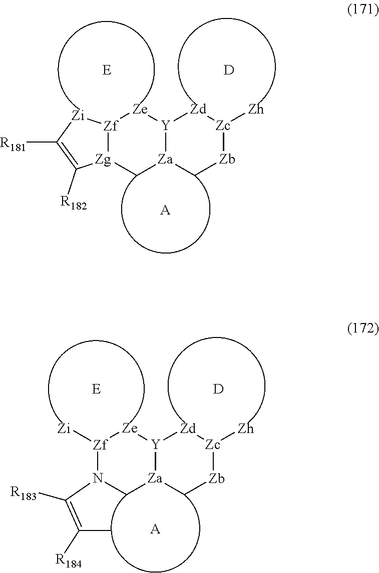

In the formulae (171) and (172):

a ring A, ring D, and ring E are each independently a cyclic structure selected from the group consisting of a substituted or unsubstituted aryl ring having 6 to 30 ring carbon atoms and a substituted or unsubstituted heterocycle having 5 to 30 ring atoms;

Za is a nitrogen atom or a carbon atom;

Zb is an oxygen atom, a sulfur atom, or NRb;

Zc is a nitrogen atom or a carbon atom;

Zd is a carbon atom or a nitrogen atom;

Ze is a carbon atom or a nitrogen atom;

Zf is a nitrogen atom or a carbon atom;

Zh is a nitrogen atom or a carbon atom;

Zi is a nitrogen atom or a carbon atom;

Y is a boron atom, a phosphorus atom, SiRh, P═O or P═S;

Rb and Rh are each independently a hydrogen atom or a substituent;

Rb and Rh as a substituent are each independently selected from the group consisting of a substituted or unsubstituted aryl group having 6 to 30 ring carbon atoms, a substituted or unsubstituted heterocyclic group having 5 to 30 ring atoms, and a substituted or unsubstituted alkyl group having 1 to 30 carbon atoms;

a combination of R₁₈₁ and R₁₈₂ are mutually bonded to form a substituted or unsubstituted monocyclic ring, mutually bonded to form a substituted or unsubstituted fused ring, or not mutually bonded;

a combination of R₁₈₃ and R₁₈₄ are mutually bonded to form a substituted or unsubstituted monocyclic ring, mutually bonded to form a substituted or unsubstituted fused ring, or not mutually bonded;

R₁₈₁ to R₁₈₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aralkyl group having 7 to 50 carbon atoms, a group represented by —Si(R₉₇₁)(R₉₇₂)(R₉₇₃), a group represented by —O—(R₉₇₄), a group represented by —S—(R₉₇₅), a group represented by —N(R₉₇₆)(R₉₇₇), a group represented by —C(═O)R₉₇₈, a group represented by —COOR₉₇₉, a halogen atom, a cyano group, a nitro group, a substituted or unsubstituted aryl group having 6 to 50 ring carbon atoms, or a substituted or unsubstituted heterocyclic group having 5 to 50 ring atoms;

R₉₇₁ to R₉₇₉ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;

when a plurality of R₉₇₁ are present, the plurality of R₉₇₁ are mutually the same or different;

when a plurality of R₉₇₂ are present, the plurality of R₉₇₂ are mutually the same or different;

when a plurality of R₉₇₃ are present, the plurality of R₉₇₃ are mutually the same or different;

when a plurality of R₉₇₄ are present, the plurality of R₉₇₄ are mutually the same or different;

when a plurality of R₉₇₅ are present, the plurality of R₉₇₅ are mutually the same or different;

when a plurality of R₉₇₆ are present, the plurality of R₉₇₆ are mutually the same or different;

when a plurality of R₉₇₇ are present, the plurality of R₉₇₇ are mutually the same or different;

when a plurality of R₉₇₈ are present, the plurality of R₉₇₈ are mutually the same or different; and

when a plurality of R₉₇₉ are present, the plurality of R₉₇₉ are mutually the same or different.

In the exemplary embodiment, the compound M1 is also preferably a compound represented by a formula (18) below.

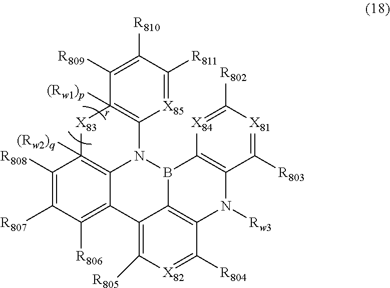

In the formula (18):

r is 0 or 1;

when r is 0, p and q are each 1, and R_{W1} and R_{W2} are present;

when r is 1, p and q are each 0, and R_{W1} and R_{W2} are not present;

X₈₁ is a nitrogen atom or CR₁₉₁;

X₈₂ is a nitrogen atom or CR₁₉₂;

X₈₃ is a single bond, an oxygen atom, a sulfur atom, Si(R₁₉₃)(R₁₉₄), C(R₁₉₅)(R₁₉₆), or BR₁₉₇;

X₈₄ is R₈₀₁, or a carbon atom bonded to X₈₅ with a single bond;

X₈₅ is R₈₁₂, or a carbon atom bonded to X₈₄ with a single bond;

at least one combination of adjacent two or more of R₁₉₁, R₁₉₂, R₁₉₃, R₁₉₄, R₁₉₅, R₁₉₆, R₁₉₇, R₈₀₁, R₈₀₂, R₈₀₃, R₈₀₄, R₈₀₅, R₈₀₆, R₈₀₇, R₈₀₈, R₈₀₉, R₈₁₀, R₈₁₁, R₈₁₂, R_{W1}, R_{W2} and R_{W3} are mutually bonded to form a substituted or unsubstituted monocyclic ring, mutually bonded to form a substituted or unsubstituted fused ring, or not mutually bonded;

R_{W3} not forming the substituted or unsubstituted monocyclic ring and not forming the substituted or unsubstituted fused ring is a substituted or unsubstituted aryl group having 6 to 50 ring carbon atoms, a substituted or unsubstituted heterocyclic group having 5 to 50 ring atoms, or a substituted or unsubstituted alkyl group having 1 to 5 carbon atoms;

R₁₉₁, R₁₉₂, R₁₉₃, R₁₉₄, R₁₉₅, R₁₉₆, R₁₉₇, R₈₀₁, R₈₀₂, R₈₀₃, R₈₀₄, R₈₀₅, R₈₀₆, R₈₀₇, R₈₀₈, R₈₀₉, R₈₁₀, R₈₁₁, R₈₁₂, R_{W1}, and R_{W2} not forming the substituted or unsubstituted monocyclic ring and not forming the substituted or unsubstituted fused ring are each independently a hydrogen atom, a deuterium atom, a group represented by —Si(R₉₈₁)(R₉₈₂)(R₉₈₃), a group represented by —O—(R₉₈₄), a group represented by —S—(R₉₈₅), a group represented by —N(R₉₈₆)(R₉₈₇), a group represented by —B(R₉₈₈)(R₉₈₉), a group represented by —OSO₂(R₉₉₀), a cyano group, a halogen atom, a substituted or unsubstituted alkyl group having 1 to 50 carbon atoms, a substituted or unsubstituted cycloalkyl group having 3 to 50 ring carbon atoms, a substituted or unsubstituted fluoroalkyl group having 1 to 50 carbon atoms, a substituted or unsubstituted thioalkoxy group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted aryl group having 6 to 50 ring carbon atoms, or a substituted or unsubstituted heterocyclic group having 5 to 50 ring atoms;

R₉₈₁ to R₉₉₀ are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms;

when a plurality of R₉₈₁ are present, the plurality of R₉₈₁ are mutually the same or different;

when a plurality of R₉₈₂ are present, the plurality of R₉₈₂ are mutually the same or different;

when a plurality of R₉₈₃ are present, the plurality of R₉₈₃ are mutually the same or different;

when a plurality of R₉₈₄ are present, the plurality of R₉₈₄ are mutually the same or different;

when a plurality of R₉₈₅ are present, the plurality of R₉₈₅ are mutually the same or different;

when a plurality of R₉₈₆ are present, the plurality of R₉₈₆ are mutually the same or different;

when a plurality of R₉₈₇ are present, the plurality of R₉₈₇ are mutually the same or different;

when a plurality of R₉₈₈ are present, the plurality of R₉₈₈ are mutually the same or different;

when a plurality of R₉₈₉ are present, the plurality of R₉₈₉ are mutually the same or different; and

when a plurality of R₉₉₀ are present, the plurality of R₉₉₀ are mutually the same or different.

### Manufacturing Method of Compound M1

The compound M1 according to the exemplary embodiment can be manufactured by application of known substitution reactions and materials depending on a target compound, in accordance with or based on synthesis methods described later in Examples.

### Specific Examples of Compound M1

Specific examples of the compound M1 in the exemplary embodiment include compounds as follows. It should however be noted that the invention is not limited to the specific examples of the compound.

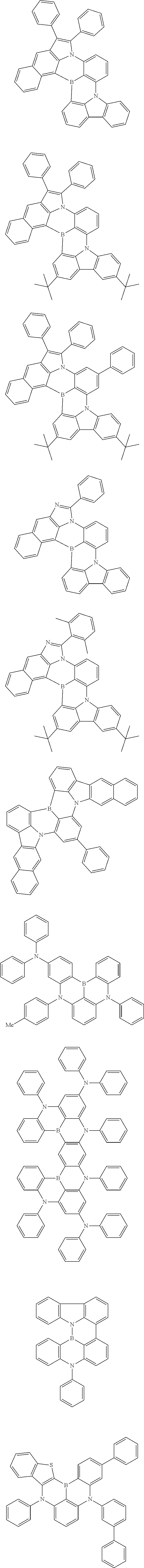

### Relationship Between Compound M3, Compound M2 and Compound M1 in Emitting Layer

In the organic EL device of the exemplary embodiment, a singlet energy S₁(M1) of the compound M1 and a singlet energy S₁(M2) of the compound M2 preferably satisfy a relationship of a numerical formula (Numerical Formula 2) below.

S₁(M2)>S₁(M1) (Numerical Formula 2)

Moreover, a singlet energy S₁(M3) of the compound M3 is preferably larger than the singlet energy S₁(M1) of the compound M1.

S₁(M3)>S₁(M1) (Numerical Formula 2A)

The singlet energy S₁(M3) of the compound M3, the singlet energy S₁(M2) of the compound M2, and the singlet energy S₁(M1) of the compound M1 preferably satisfy a relationship of a numerical formula (Numerical Formula 2B) below.

S₁(M3)>S₁(M2)>S₁(M1) (Numerical Formula 2B)

It is preferable that mainly the fluorescent compound M1 emits light in the emitting layer when the organic EL device of the exemplary embodiment emits light.

The organic EL device of the exemplary embodiment preferably emits red light or green light.

### Content Ratios of Compounds in Emitting Layer

Content ratios of the compound M3, the compound M2, and the compound

M1 in the emitting layer preferably fall within the exemplary range below.

The content ratio of the compound M3 is preferably in a range from 10 mass % to 80 mass %.

The content ratio of the compound M2 is preferably in a range from 10 mass % to 80 mass %, more preferably in a range from 10 mass % to 60 mass %, further preferably in a range from 20 mass % to 60 mass %.

The content ratio of the compound M1 is preferably in a range from 0.01 mass % to 10 mass %, more preferably in a range from 0.01 mass % to 5 mass %, further preferably in a range from 0.01 mass % to 1 mass %.

The upper limit of a total of the content ratios of the compound M3, the compound M2, and the compound M1 in the emitting layer is 100 mass %. It should be noted that the exemplary embodiment does not exclude that the emitting layer contains a material other than the compound M3, the compound M2, and the compound M1.

The emitting layer may contain a single type of the compound M3 or may contain two or more types of the compound M3. The emitting layer may contain a single type of the compound M2 or may include two or more types of the compound

M2. The emitting layer may, contain a single type of the compound M1 or may contain two or more types of the compound M1.

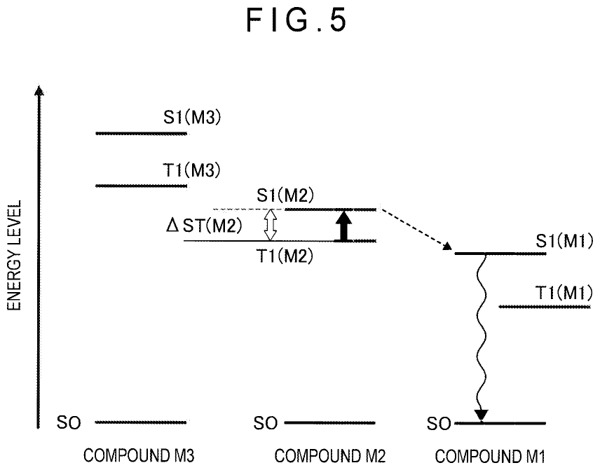

FIG. 5 shows an example of a relationship between energy levels of the compound M3, the compound M2, and the compound M1 in the emitting layer. In FIG. 5, S0 represents a ground state. S1(M1) represents the lowest singlet state of the compound M1. T1(M1) represents the lowest triplet state of the compound M1. S1(M2) represents the lowest singlet state of the compound M2. T1(M2) represents the lowest triplet state of the compound M2. S1(M3) represents the lowest singlet state of the compound M3. T1(M3) represents the lowest triplet state of the compound M3. A dashed arrow directed from S1(M2) to S1(M1) in FIG. 5 represents Førster energy transfer from the lowest singlet state of the compound M2 to the lowest singlet state of the compound M1.

As shown in FIG. 5, when a compound having a small ΔST(M2) is used as the compound M2, inverse intersystem crossing from the lowest triplet state T1(M2) to the lowest singlet state S1(M2) can be caused by a heat energy. Subsequently, Førster energy transfer from the lowest singlet state S1(M2) of the compound M2 to the compound M1 occurs to generate the lowest singlet state S1(M1). Consequently, fluorescence from the lowest singlet state S1(M1) of the compound M1 can be observed. It is inferred that the internal quantum efficiency can be theoretically raised up to 100% also by using delayed fluorescence by the TADF mechanism.

According to the second exemplary embodiment, an organic EL device having a high performance, especially high luminous efficiency, can be provided. The organic EL device according to the second exemplary embodiment is applicable to an electronic device such as a display device and a light-emitting device.

### Third Exemplary Embodiment

### Electronic Device

An electronic device according to the present exemplary embodiment is installed with any one of the organic EL devices according to the above exemplary embodiments. Examples of the electronic device include a display device and a light-emitting device. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited by the above-described exemplary embodiments but includes any modification and improvement as long as such modification and improvement are compatible with the invention.

For instance, the emitting layer is not limited to a single layer, but may be provided by laminating a plurality of emitting layers. When the organic EL device has a plurality of emitting layers, it is only required that at least one of the emitting layers satisfies the conditions described in the above exemplary embodiments. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers are mutually adjacently provided, or form a so-called tandem organic EL device, in which a plurality of emitting units are layered via an intermediate layer.

For instance, in an exemplary embodiment, a blocking layer is provided adjacent to at least one of a side near the anode or a side near the cathode of the emitting layer. The blocking layer is preferably provided in contact with the emitting layer to block at least any of holes, electrons, and excitons.

For instance, when the blocking layer is provided in contact with the cathode-side of the emitting layer, the blocking layer permits transport of electrons, and blocks holes from reaching a layer provided near the cathode (e.g., the electron transporting layer) beyond the blocking layer. When the organic EL device includes the electron transporting layer, the blocking layer is preferably disposed between the emitting layer and the electron transporting layer.

When the blocking layer is provided in contact with the anode-side of the emitting layer, the blocking layer permits transport of holes, but blocks electrons from reaching a layer provided near the anode (e.g., the hole transporting layer) beyond the blocking layer. When the organic EL device includes the hole transporting layer, the blocking layer is preferably disposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

Specific structure, shape and the like of the components in the invention may be designed in any manner as long as an object of the invention can be achieved.

### EXAMPLES

Example(s) of the invention will be described below. However, the invention is not limited to Example(s).

### Compounds

A structure of the compound represented by the formula (2) used for manufacturing organic EL devices in Examples 1 and 2 is shown below.

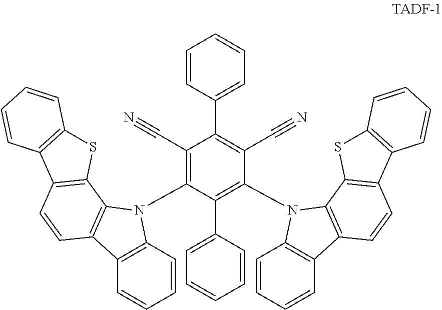 [SMILES: N#Cc1c(-c2ccccc2)c(C#N)c(-n2c3ccccc3c3ccc4c5ccccc5sc4c32)c(-c2ccccc2)c1-n1c2ccccc2c2ccc3c4ccccc4sc3c21]

A structure of the compound represented by the formula (3) used for manufacturing the organic EL devices in Examples 1 and 2 is shown below.

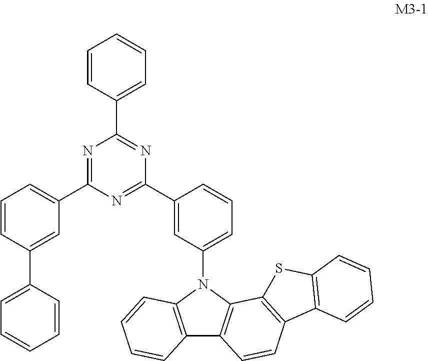 [SMILES: c1ccc(-c2cccc(-c3nc(-c4ccccc4)nc(-c4cccc(-n5c6ccccc6c6ccc7c8ccccc8sc7c65)c4)n3)c2)cc1]

Structures of comparative compounds used for manufacturing organic EL devices in Comparatives 1 to 3 are shown below.

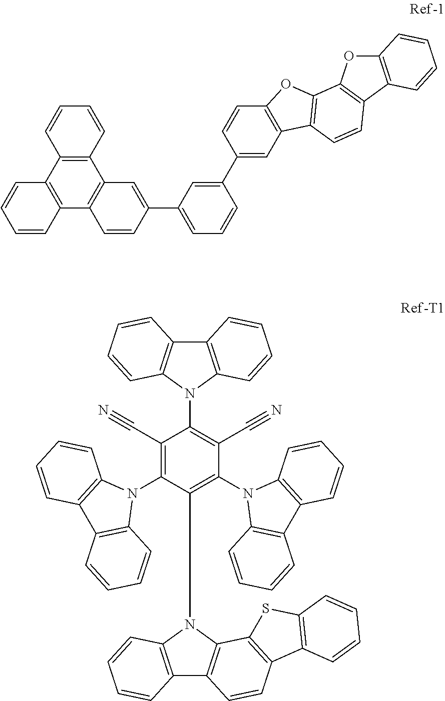 [SMILES: c1cc(-c2ccc3oc4c(ccc5c6ccccc6oc54)c3c2)cc(-c2ccc3c4ccccc4c4ccccc4c3c2)c1 ; N#Cc1c(-n2c3ccccc3c3ccccc32)c(C#N)c(-n2c3ccccc3c3ccccc32)c(-n2c3ccccc3c3ccc4c5ccccc5sc4c32)c1-n1c2ccccc2c2ccccc21]

Structures of other compounds used for manufacturing the organic EL devices in Examples 1 and 2 and Comparatives 1 to 3 are shown below.

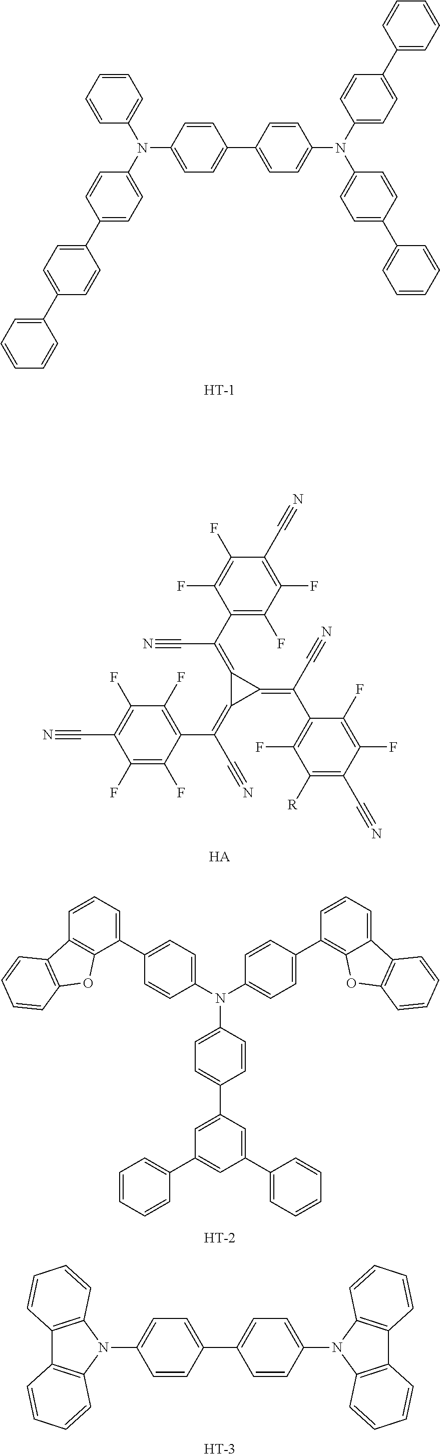

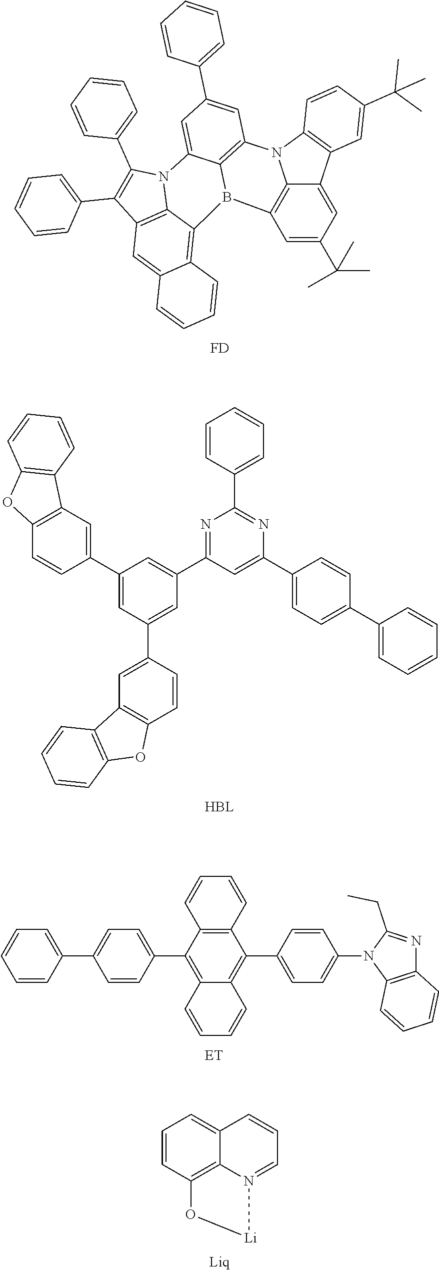

### Manufacture 1 of Organic EL Device

The organic EL devices were manufactured and evaluated as follows.

### Example 1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for one minute. A film thickness of ITO was 135 nm.

After the glass substrate having the transparent electrode line was cleaned, the glass substrate was mounted on a substrate holder of a vacuum evaporation apparatus. Firstly, a compound HT-1 and a compound HA were co-deposited on a surface of the glass substrate where the transparent electrode line was provided in a manner to cover the transparent electrode, thereby forming a 10-nm-thick hole injecting layer. The concentrations of the compound HT-1 and the compound HA in the hole injecting layer were 97 mass % and 3 mass %, respectively.

Next, the compound HT-1 was vapor-deposited on a hole injecting layer to form a 110-nm-thick first hole transporting layer.

Next, a compound HT-2 was vapor-deposited on the first hole transporting layer to form a 5-nm-thick second hole transporting layer.

Next, a compound HT-3 was vapor-deposited on the second hole transporting layer to form a 5-nm-thick electron blocking layer.

Next, a compound M3-1 as the compound M3 and a compound TADF-1 as the compound M2 were co-deposited on the electron blocking layer to form a 25-nm emitting layer. The concentrations of the compound M3-1 and the compound TADF-1 in the emitting layer were 75 mass % and 25 mass %, respectively.

Next, a compound HBL was vapor-deposited on the emitting layer to form a 5-nm-thick hole blocking layer.

Next, a compound ET was vapor-deposited on the hole blocking layer to form a 20-nm-thick first electron transporting layer.

Next, the compound ET and the compound Liq were co-deposited on the first electron transporting layer to form a 20-nm-thick second electron transporting layer. The concentrations of the compound ET and the compound Liq in the second electron transporting layer were 50 mass % and 50 mass %, respectively.

Next, ytterbium (Yb) was vapor-deposited on the second electron transporting layer to form a 1-nm-thick electron injectable electrode (cathode).

Subsequently, metal aluminum (Al) was vapor-deposited on the electron injectable electrode to form an 80-nm-thick metal Al cathode.

A device arrangement of the organic EL device of Example 1 is roughly shown as follows.

ITO(135)/HT-1:HA(10,97%:3%)/HT-1(110)/HT-2(5)/HT-3(5)/M3-1:TADF-1(25,75%:25%)/HBL(5)/ET(20)/ET:Lig(20,50%:50%)/Yb(1)/Al(80)

Numerals in parentheses represent a film thickness (unit: nm).

The numerals (97%:3%) represented by percentage in the same parentheses indicate a ratio (mass %) between the compound HT-1 and the compound HA in the hole injecting layer. The numerals (75%:25%) represented by percentage in the same parentheses indicate a ratio (mass %) between the compound M3-1 and the compound TADF-1 in the emitting layer. The numerals (50%:50%) represented by percentage in the same parentheses indicate a ratio (mass %) between compound ET and the compound Liq in the second electron transporting layer. Similar notations apply to the description below.

### Comparative 1

The organic EL device in Comparative 1 was manufactured in the same manner as in Example 1 except that the compound M3-1 in the emitting layer of Example 1 was replaced by a comparative compound Ref-1 shown in Table 1.

### Comparative 2

The organic EL device in Comparative 2 was manufactured in the same manner as in Example 1 except that the compound TADF-1 in the emitting layer of Example 1 was replaced by a comparative compound Ref-T1 shown in Table 1.

### Evaluation of Organic EL Devices

The manufactured organic EL devices were evaluated as follows. Evaluation results are shown in Tables 1 and 2. Although the compound Ref-1 used in Comparatives 1 and 3 do not correspond to the compound M3, the comparative compound Ref-1 is shown in the same column as the compound M3-1 in Examples 1 and 2 for convenience. Although the compound Ref-T1 used in Comparatives 2 and 4 do not correspond to the compound M2, the comparative compound Ref-T1 is shown in the same column as the compound TADF-1 in Examples 1 and 2 for convenience. Singlet energies Si of the respective compound M3, compound M2, and compound M1 used in the emitting layer of each Example are also shown in Tables 1 and 2.

### Maximum Peak Wavelength λₚ

Voltage was applied on the organic EL devices so that a current density was mA/cm², where spectral radiance spectrum was measured by a spectroradiometer CS-2000 (manufactured by Konica Minolta, Inc.). The maximum peak wavelength λₚ (unit: nm) was obtained from the measured spectral radiance spectrum.

### External Quantum Efficiency EQE

Voltage was applied on the organic EL devices so that a current density was mA/cm², where spectral radiance spectrum was measured by a spectroradiometer CS-2000 (manufactured by Konica Minolta, Inc.). The external quantum efficiency EQE (unit: %) was calculated based on the obtained spectral-radiance spectra, assuming that the spectra was provided under a Lambertian radiation.

TABLE 1

| | Emitting Layer | | | | | | Device | |
|---|---|---|---|---|---|---|---|---|
| | Compound M3 | | Compound M2 | | | | Evaluation | |
| | | S₁ | | S₁ | Delay/ | ΔST | λp | EQE |
| | Name | [eV] | Name | [eV] | Prompt | [eV] | [nm] | [%] |
| Example 1 | M3-1 | 3.08 | TADF-1 | 2.66 | >0.05 | <0.01 | 528 | 19.1 |
| Comparative 1 | Ref-1 | 3.68 | TADF-1 | 2.66 | >0.05 | <0.01 | 533 | 15.5 |
| Comparative 2 | M3-1 | 3.08 | Ref-T1 | 2.57 | >0.05 | <0.01 | 549 | 14.0 |

### Manufacture 2 of Organic EL Device

An organic EL device was manufactured as follows. The manufactured organic EL device was evaluated in the same manner as the above.

### Example 2

An organic EL device in Example 2 was manufactured in the same manner as in Example 1 except that the emitting layer was formed as follows in place of forming the emitting layer in Example 1. In Example 2, the compound M3-1 as the compound M3, the compound TADF-1 as the compound M2, and the compound FD as the compound M1 were co-deposited on the electron blocking layer to form a 25-nm emitting layer. Concentrations of the compound M3-1, the compound TADF-1, and the compound FD in the emitting layer in Example 2 were 75 mass %, 24 mass %, and 1 mass %, respectively.

### Comparative 3

An organic EL device in Comparative 3 was manufactured in the same manner as in Example 2 except that the compound M3-1 contained in the emitting layer in Example 2 was replaced by the comparative compound Ref-1 shown in Table 2.

### Comparative 4

An organic EL device in Comparative 4 was manufactured in the same manner as in Example 2 except that the compound TADF-1 contained in the emitting layer in Example 2 was replaced by the comparative compound Ref-T1 shown in Table 2.

TABLE 2

| | Emitting Layer | | | | | | | | | Device | |
|---|---|---|---|---|---|---|---|---|---|---|---|
| | Compound M3 | | Compound M2 | | | | Compound M1 | | | Evaluation | |
| | | S₁ | | S₁ | Delay/ | ΔST | | S₁ | λ | λp | EQE |
| | Name | [eV] | Name | [eV] | Prompt | [eV] | Name | [eV] | [nm] | [nm] | [%] |
| Example 2 | M3-1 | 3.08 | TADF-1 | 2.66 | >0.05 | <0.01 | FD | 2.39 | 512 | 520 | 15.6 |
| Comparative 3 | Ref-1 | 3.68 | TADF-1 | 2.66 | >0.05 | <0.01 | FD | 2.39 | 512 | 521 | 8.6 |
| Comparative 4 | M3-1 | 3.08 | Ref-T1 | 2.57 | >0.05 | <0.01 | FD | 2.39 | 512 | 520 | 11.2 |
| Example 2 | M3-1 | 3.08 | TADF-1 | 2.66 | >0.05 | <0.01 | FD | 2.39 | 512 | 520 | 15.6 |
| Comparative 3 | Ref-1 | 3.68 | TADF-1 | 2.66 | >0.05 | <0.01 | FD | 2.39 | 512 | 521 | 8.6 |
| Comparative 4 | M3-1 | 3.08 | Ref-T1 | 2.57 | >0.05 | <0.01 | FD | 2.39 | 512 | 520 | 11.2 |

### Evaluation of Compounds

### Thermally Activated Delayed Fluorescence

### Delayed Fluorescence of Compound TADF-1

Delayed fluorescence properties were checked by measuring transient photoluminescence (PL) using a device shown in FIG. 2. The compound TADF-1 was dissolved in toluene to prepare a dilute solution with an absorbance of 0.05 or less at the excitation wavelength to eliminate the contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the above sample solution was measured with a spectrofluorometer FP-8600 (manufactured by JASCO Corporation), and the fluorescence spectrum of a 9,10-diphenylanthracene ethanol solution was measured under the same conditions. Using the fluorescence area intensities of both spectra, the total fluorescence quantum yield is calculated by an equation (1) in Morris et al. J. Phys. Chem. 80 (1976) 969.

Prompt emission was observed immediately when the excited state was achieved by exciting the compound TADF-1 with a pulse beam (i.e., a beam emitted from a pulse laser) having a wavelength to be absorbed by the compound TADF-1, and Delay emission was observed not immediately when the excited state was achieved but after the excited state was achieved. The delayed fluorescence in Examples means that an amount of Delay Emission is 5% or more with respect to an amount of Prompt Emission. Specifically, provided that the amount of Prompt emission is denoted by X_{P} and the amount of Delay emission is denoted by X_{D}, the delayed fluorescence means that a value of X_{D}/X_{P} is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

It was confirmed that the amount of Delay Emission was 5% or more with respect to the amount of Prompt Emission in the compound TADF-1.

Specifically, a value of X_{D}/X_{P} in the compound TADF-1 was confirmed to be 0.05 or more.

In Tables, the notation “>0.05” indicates that the value of X_{D}/X_{P} exceeded 0.05.

### Delayed Fluorescence of Comparative Compound Ref-T1

Delayed fluorescence of the comparative compound Ref-T1 was checked in the same manner as the above except that the compound TADF-1 was replaced by the comparative compound Ref-T1.

A value of X_{D}/X_{P} in the comparative compound Ref-T1 was 0.05 or more.

### Singlet Energy S₁

Singlet energy S₁ of each of the compound M3-1, the compound TADF-1, the compound FD, the comparative compound Ref-1 and the comparative compound Ref-T1 was measured according to the above-described solution method. Tables 1 and 2 show measurement results.

### ΔST

T_{77K} of each of the compound TADF-1 and the compound Ref-T1 was measured. T_{77K} was measured by the measurement method of the energy gap T_{77K} described in “Relationship between Triplet Energy and Energy Gap at 77K.”

ΔST was calculated based on the measured lowest singlet energy Si and energy gap T_{77K} at 77K. Values of ΔST of the compounds TADF-1 and Ref-T1 are shown in Tables 1 and 2. In Tables, the notation “<0.01” indicates that ΔST was less than 0.01 eV.

### Maximum Peak Wavelength A of Compounds

A 5-μmol/L toluene solution of each of the compounds (measurement target) was prepared and put in a quartz cell. A fluorescence spectrum (ordinate axis: fluorescence intensity, abscissa axis: wavelength) of each of the samples was measured at a normal temperature (300K). In Examples, a fluorescence spectrum was measured with a spectrophotofluorometer (manufactured by Hitachi High-Tech Science Corporation: F-7000). It should be noted that the fluorescence spectrum measuring device may be different from the above device. A peak wavelength of a fluorescence spectrum, a luminous intensity of which is the maximum in the fluorescence spectrum, was defined as the maximum peak wavelength A.

### SYNTHESIS OF COMPOUNDS

### Synthesis Example 1: Synthesis of Compound TADF-1

A synthesis method of the compound TADF-1 will be described below.

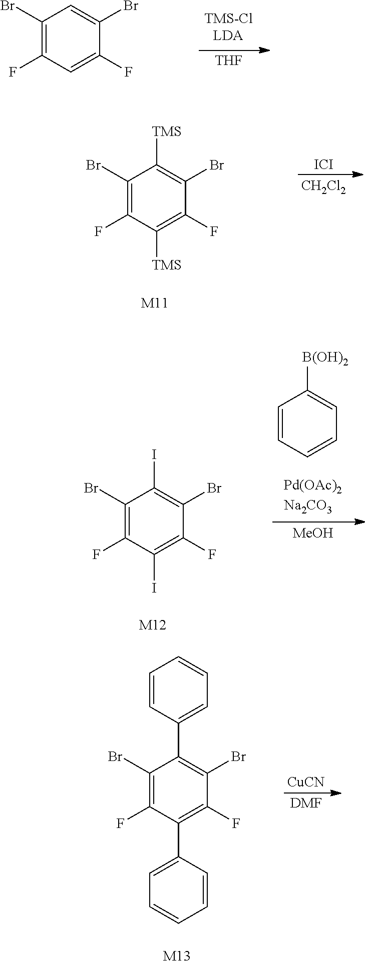

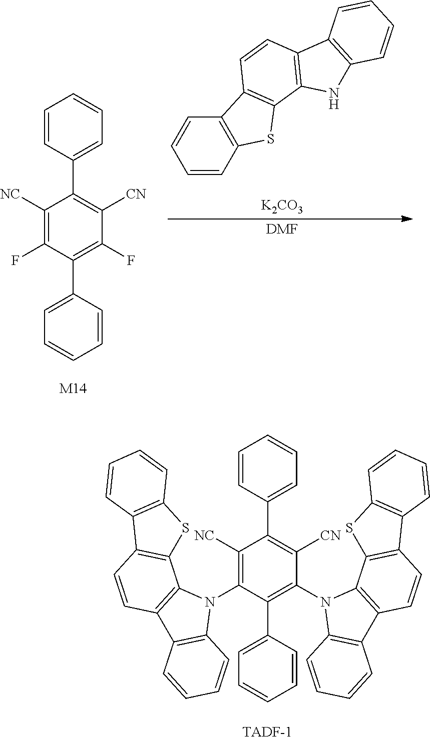

Under nitrogen atmosphere, 1,5-dibromo-2,4-difluorobenzene (50 g, 184 mmol), chlorotrimethylsilane (60 g, 552 mmol), and THF (200 mL) were put into a 1000-mL three-necked flask. The material in the three-necked flask was cooled to −78 degrees C. in a dry ice/acetone bath. Subsequently, 230 mL of lithium diisopropyl amide (2M, THF solution) was dropped into the material. The material was stirred at −78 degrees C. for 2 hours, then returned to a room temperature, and further stirred for 2 hours. After stirring, water (200 mL) was added into the three-necked flask. Subsequently, an organic layer was extracted with acetic ether. The extracted organic layer was washed with water and a saline solution and dried with magnesium sulfate. Then, a solvent was removed by a rotary evaporator under reduced pressure. The obtained intermediate M11 (73 g, 175 mmol, a yield of 95%) was not purified and used for a next reaction. Chlorotrimethylsilane is sometimes abbreviated as TMS-Cl. TMS in a formula representing the intermediate M11 is a trimethylsilyl group. LDA is an abbreviation for lithium diisopropyl amide.

Under nitrogen atmosphere, the intermediate M11 (73 g, 175 mmol) and dichloromethane (200 mL) were put into a 1000-mL eggplant flask. Iodine monochloride (85 g, 525 mmol) was dissolved in dichloromethane (200 mL) and dropped therein at 0 degree C. Subsequently, the mixture was stirred at 40 degrees C. for 4 hours. After stirring, the mixture was returned to a room temperature and added with a saturated aqueous solution of sodium hydrogen sulfite (100 mL). Then, an organic layer was extracted with dichloromethane. The extracted organic layer was washed with water and a saline solution. The washed organic layer was dried with magnesium sulfate. The dried organic layer was condensed by a rotary evaporator. A compound obtained through condensation was purified by silica-gel column chromatography to obtain an intermediate M12 (65 g, 124 mmol, a yield of 71%).

Under nitrogen atmosphere, the intermediate M12 (22 g, 42 mmol), phenylboronic acid (12.8 g, 105 mmol), palladium acetate (0.47 g, 2.1 mmol), sodium carbonate (22 g, 210 mmol), and methanol (150 mL) were put into a 500-mL three-necked flask and stirred for four hours at 80 degrees C. After stirring, the reaction solution was left to be cooled to a room temperature. Subsequently, an organic layer was extracted with acetic ether. The extracted organic layer was washed with water and a saline solution. The washed organic layer was condensed by a rotary evaporator. A compound obtained through condensation was purified by silica-gel column chromatography to obtain an intermediate M13 (10 g, 24 mmol, a yield of 56%). The structure of the purified compound was identified by ASAP/MS. ASAP/MS is an abbreviation for Atmospheric Pressure Solid Analysis Probe Mass Spectrometry.

Under nitrogen atmosphere, the intermediate M13 (10 g, 24 mmol), copper cyanide (10.6 g, 118 mmol), and DMF (15 mL) were put into a 200-mL three-necked flask and heated at 150 degrees C. for eight hours with stirring. After stirring, the reaction solution was cooled to a room temperature and then poured into ammonia water (10 mL). Next, an organic layer was extracted with methylene chloride. The extracted organic layer was washed with water and a saline solution. The washed organic layer was dried with magnesium sulfate. After drying, a solvent was removed by a rotary evaporator under reduced pressure. A compound obtained through removal under reduced pressure was purified by silica-gel column chromatography to obtain an intermediate M14 (5.8 g, 18.34 mmol, a yield of 78%). DMF is an abbreviation for N,N-dimethylformamide.

Under nitrogen atmosphere, the intermediate M14 (1.0 g, 3.2 mmol), 12H-[1]Benzothieno[2,3-a]carbazole (1.9 g, 7 mmol), potassium carbonate (1.3 g, 9.50 mmol), and DMF (30 mL) were put into a 100-mL three-necked flask and stirred at 120 degrees C. for six hours. After stirring, the deposited solid was collected by filtration and purified by silica-gel column chromatography to obtain a compound TADF-1 (1.8 g, 2.2 mmol, a yield of 69%). The obtained compound was identified as the compound TADF-1 by analysis according to ASAP-MS.

### Synthesis Example 2: Synthesis of Compound FD

A synthesis method of a compound FD will be described below.

### Manufacture of Intermediate M21

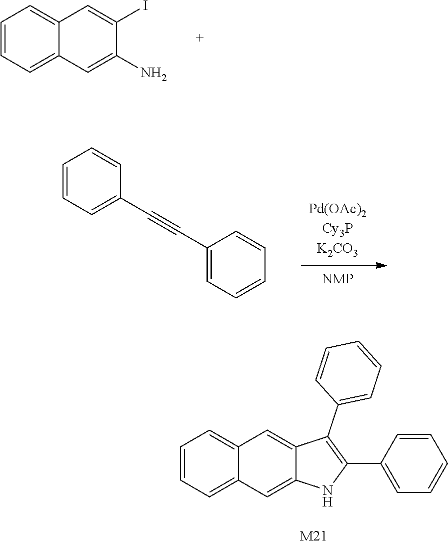

Under argon atmosphere, a mixture of 2-amino-3-iodonaphthalene (4.28 g), 1,2-diphenylacetylene (3.40 g), palladium(II) acetate (178 mg), tricyclohexylphosphine (446 mg), potassium carbonate (5.49 g), and N-methyl pyrrolidone (360 mL) was stirred at 110 degrees C. for five hours. The obtained mixture was cooled to the room temperature. N-methyl pyrrolidone was partially distilled under reduced pressure. Subsequently, the obtained mixture was diluted with t-butylmethylether and added to water. An aqueous layer was extracted with t-butylmethylether. An organic layer was washed with saturated saline solution and subsequently dried with magnesium sulfate, and a solvent was distilled under reduced pressure. The obtained residue was purified by silica-gel column chromatography to obtain an intermediate M21 (2.78 g, 55%). In a reaction scheme, Pd(OAc)₂ represents palladium(II) acetate, Cy₃P represents tricyclohexylphosphine, and NMP represents N-methyl pyrrolidone.

### Manufacture of Intermediate M22

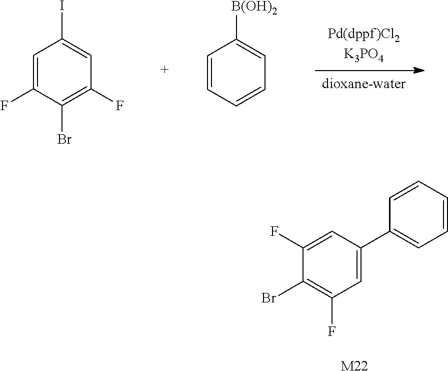

under argon atmosphere, a mixture of 2-bromo-1,3-difluoro-5-iodobenzene (47.8 g), phenylboronic acid (18.29 g), tripotassium phosphate (39.8 g), [1,1-bis(diphenyl phosphino) ferrocene]palladium(II) dichloride (1.09 g), 1,4-dioxane (250 mL), and water (125 mL) was stirred at the room temperature for four hours. Toluene (250 mL) and water (200 mL) were added to the obtained mixture to extract an aqueous layer with toluene. An organic layer was washed with a saturated saline solution and subsequently dried with magnesium sulfate, and a solvent was distilled. The obtained residue was purified by silica-gel column chromatography to obtain an intermediate M22 (35.1 g, 87%). In a reaction scheme, Pd(dppf)C12 represents [1,1-bis(diphenylphosphino) ferrocene]palladium(II) dichloride.

### Manufacture of Intermediate M23

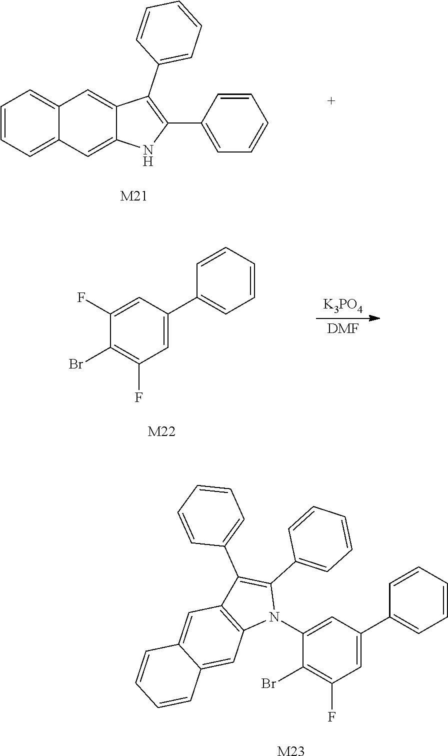

Under argon atmosphere, a mixture of the intermediate M21 (6.39 g), the intermediate M22 (10.76 g), tripotassium phosphate (21.23 g), and dimethylformamide (140 mL) was stirred at 105 degrees C. for 48 hours. Dimethylformamide was partially distilled under reduced pressure, and subsequently, to which water was added, and extraction using t-butylmethylether was performed. An organic layer was washed with saturated saline solution and subsequently dried with magnesium sulfate, and a solvent was distilled under reduced pressure. The obtained residue was purified by silica-gel column chromatography to obtain an intermediate M23 (6.2 g, 55%). In a reaction scheme, DMF represents dimethylformamide.

### Manufacture of Intermediate M24

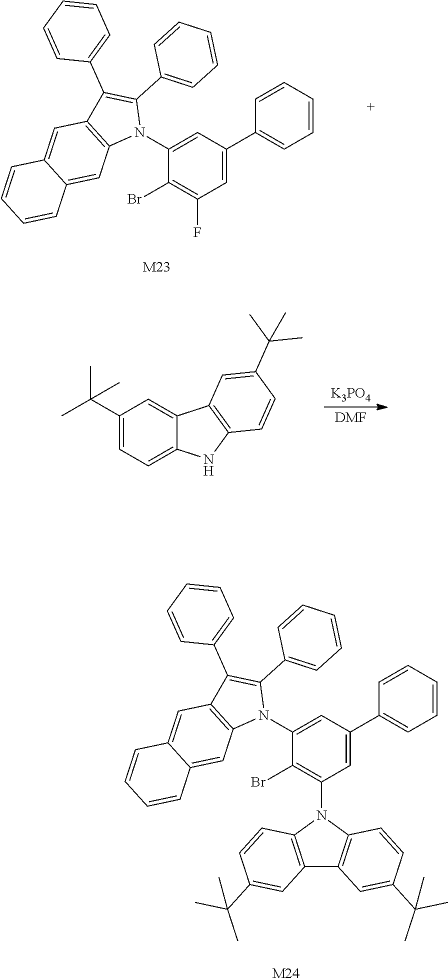

Under argon atmosphere, a mixture of the intermediate M23 (6.14 g), 3,6-di-tert-butyl-9H-carbazole (3.32 g), tripotassium phosphate (6.88 g), and dimethylformamide (96 mL) was stirred at 105 degrees C. for 20 hours. Dimethylformamide was partially distilled under reduced pressure. The obtained mixture was put into water (150 mL) and stirred. The deposited solid was collected by filtration, washed with water, and subsequently dried under reduced pressure. Further, the obtained solid was suspended in ethanol (220 mL) and heated for reflux for one hour. Subsequently, the solid was collected by filtration to obtain an intermediate M24 (7.31 g, 82%).

### Manufacture of Compound FD

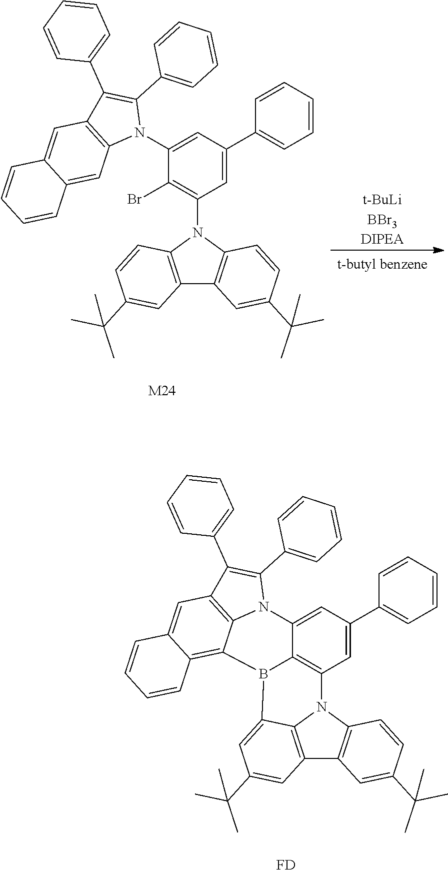

Under argon atmosphere, the intermediate M24 (2.23 g) was added to tert-butyl benzene (33 mL) and cooled to −20 degrees C., subsequently, to which 1.9M pentane solution (2.8 mL) of tert-butyl lithium was added dropwise. After the dropwise addition, the obtained mixture was raised in temperature to 70 degrees C. and stirred for 30 minutes. Subsequently, a component having a boiling point lower than that of tert-butyl benzene was distilled under reduced pressure. The obtained mixture was cooled to −55 degrees C., added with boron tribromide (0.57 mL), raised in temperature to a room temperature, stirred at the room temperature until exotherm subsided, subsequently raised in temperature to 130 degrees C., and stirred overnight. After tert-butylbenzene was distilled under reduced pressure, the residue was purified by flash chromatography to obtain an orange compound (350 mg). As a result of mass spectrometry, this orange compound was a target substance (compound FD) and had 757.4 [M+11]⁺ while a molecular weight was 756.8. In a reaction scheme, t-BuLi represents tert-butyllithium and DIPEA represents N,N-diisopropylethylamine.

What is claimed is:

1. An organic electroluminescence device comprising:
an anode;
a cathode;
an emitting layer provided between the anode and the cathode, wherein
the emitting layer comprises a delayed fluorescent compound M2 represented by a formula (2) below and a compound M3 represented by a formula (3) below, and
a singlet energy S₁(M2) of the compound M2 and a singlet energy S₁(M3) of the compound M3 satisfy a relationship of a numerical formula (Numerical Formula 1) below, 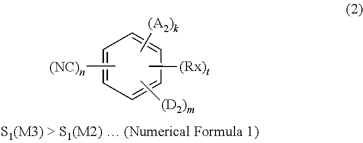
where:
k is 1, 2, 3, or 4;
m is 1, 2, 3, or 4;
n is 1 or 2;
t is 0, 1, 2, or 3;
k+m+n+t is 6;
when t is 2 or 3, a plurality of Rx are mutually the same or different;
A₂ is a group represented by a formula (21) below;
when k is 2, 3, or 4, a plurality of A₂ are mutually the same or different;
D₂ is a group represented by a formula (22) below; and
when m is 2, 3, or 4, a plurality of D₂ are mutually the same or different; and
CN is a cyano group, 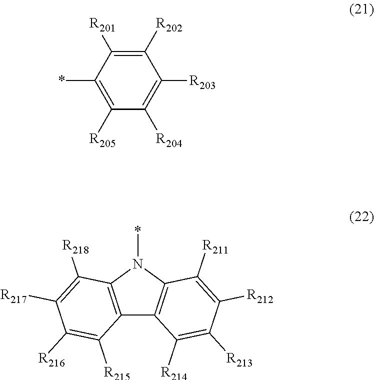
where
in the formula (21):
at least one combination of adjacent two or more of R₂₀₁ to R₂₀₅ are mutually bonded to form a substituted or unsubstituted monocyclic ring, mutually bonded to form a substituted or unsubstituted fused ring, or not mutually bonded; and
in the formula (22):
at least one combination of adjacent two or more of R₂₁₁ to R₂₁₈ are mutually bonded to form a substituted or unsubstituted monocyclic ring, mutually bonded to form a substituted or unsubstituted fused ring, or not mutually bonded;
in each of the formulae (21) and (22) represents a bonding position to a benzene ring in the formula (2); and
Rx in the formula (2), R₂₀₁ to R₂₀₅ not forming the substituted or unsubstituted monocyclic ring and not forming the substituted or unsubstituted fused ring in the formula (21), and R₂₁₁ to R₂₁₈ not forming the substituted or unsubstituted monocyclic ring and not forming the substituted or unsubstituted fused ring in the formula (22) are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms, 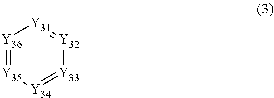
where:
Y₃₁ to Y₃₆ are each independently CR₃ or a nitrogen atom;
two or more of Y₃₁ to Y₃₆ are each a nitrogen atom;
when a plurality of R₃ are present, at least one combination of adjacent two or more of the plurality of R₃ are mutually bonded to form a substituted or unsubstituted monocyclic ring, mutually bonded to form a substituted or unsubstituted fused ring, or not mutually bonded; and
R₃ not forming the substituted Or unsubstituted monocyclic ring and not forming the substituted or unsubstituted fused ring is each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, a substituted or unsubstituted heterocyclic group having 5 to 50 ring atoms, or a group represented by a formula (3A), 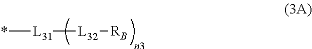
where:
R_{B} is a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms;
when a plurality of R_{B} are present, the plurality of R_{B} are mutually the same or different;
L₃₁ is: a single bond;
a substituted or unsubstituted arylene group having 6 to 50 ring carbon atoms, or a trivalent, tetravalent, pentavalent, or hexavalent group derived from the arylene group;
a substituted or unsubstituted divalent heterocyclic group having 5 to 50 ring atoms, or a trivalent, tetravalent, pentavalent, or hexavalent group derived from the heterocyclic group; or
a divalent group formed by bonding two groups selected from the group consisting of a substituted or unsubstituted arylene group having 6 to 50 ring carbon atoms and a substituted or unsubstituted divalent heterocyclic group having 5 to 50 ring atoms, or a trivalent, tetravalent, pentavalent, or hexavalent group derived from the divalent group;
L₃₂ is a single bond, a substituted or unsubstituted arylene group having 6 to 50 ring carbon atoms, or a substituted or unsubstituted divalent heterocyclic group having 5 to 50 ring atoms;
n₃ is 1, 2, 3, 4, or 5;
when L₃₁ is a single bond, n₃ is 1 and L₃₂ is bonded to a carbon atom in a six-membered ring in the formula (3);
when a plurality of L₃₂ are present, the plurality of L₃₂ are mutually the same or different;
* represents a bonding position to a carbon atom in a six-membered ring in the formula (3); and
when the compound M3 is a compound Mx3 represented by a formula (301) below and a singlet energy Si(Mx3) of the compound Mx3 is larger than the singlet energy S₁(M2) of the compound M2, the emitting layer does not comprise a compound having a larger singlet energy than the singlet energy S₁(M2) of the compound M2, other than the compound Mx3, 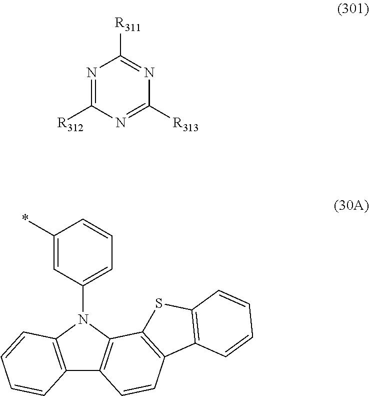
where:
R₃₁₁ is a phenyl structure;
R₃₁₂ is a biphenyl structure;
R₃₁₃ is a structure represented by the formula (30A);
in the compound M2 and the compound M3, R₉₀₁, R₉₀₂, R₉₀₃, R₉₀₄, R₉₀₅, R₉₀₆, R₉₀₇, R₉₀₈, R₉₀₉, R₉₃₁, R₉₃₂, R₉₃₃, R₉₃₄, R₉₃₅, R₉₃₆, and R₉₃₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₉₀₈ are present, the plurality of R₉₀₈ are mutually the same or different;
when a plurality of R₉₀₉ are present, the plurality of R₉₀₉ are mutually the same or different;
when a plurality of R₉₃₁ are present, the plurality of R₉₃₁ are mutually the same or different;
when a plurality of R₉₃₂ are present, the plurality of R₉₃₂ are mutually the same or different;
when a plurality of R₉₃₃ are present, the plurality of R₉₃₃ are mutually the same or different;
when a plurality of R₉₃₄ are present, the plurality of R₉₃₄ are mutually the same or different;
when a plurality of R₉₃₅ are present, the plurality of R₉₃₅ are mutually the same or different;
when a plurality of R₉₃₆ are present, the plurality of R₉₃₆ are mutually the same or different; and
when a plurality of R₉₃₇ are present, the plurality of R₉₃₇ are mutually the same or different.

2. The organic electroluminescence device according to claim 1, wherein n in the formula (2) is 2.

3. The organic electroluminescence device according to claim 1, wherein the compound M2 is a compound represented by a formula (201) below, 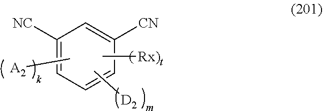
where:
A₂, D₂ and Rx respectively represent the same as A₂, D₂ and Rx in the formula (2);
k is 1, 2, or 3;
m is 1, 2, or 3;
t is 0, 1, or 2; and
k+m+t is 4.

4. The organic electroluminescence device according to claim 1, wherein m in the compound M2 is 2.

5. The organic electroluminescence device according to claim 1, wherein the compound M2 is a compound represented by a formula (211) below, 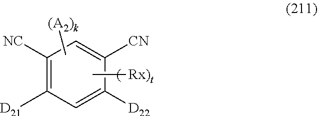
where:
D₂₁ and D₂₂ each independently represent the same as D₂;
A₂ and Rx respectively represent the same as A₂ and Rx in the formula (2);
k is 1 or 2;
t is 0 or 1; and
k+t is 2.

6. The organic electroluminescence device according to claim 1, wherein k in the compound M2 is 1 or 2.

7. The organic electroluminescence device according to claim 1, wherein k in the compound M2 is 2.

8. The organic electroluminescence device according to claim 6, wherein the compound M2 is a compound represented by a formula (202) or a formula (203) below, 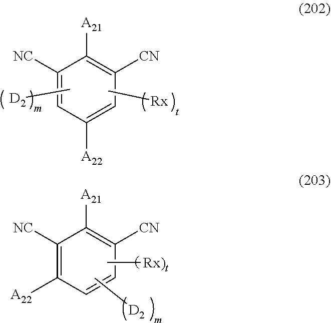
where:
A₂₁ and A₂₂ each independently represent the same as A₂;
D₂ and Rx respectively represent the same as D₂ and Rx in the formula (2);
m is 1 or 2;
t is 0 or 1; and
m+t is 2.

9. The organic electroluminescence device according to claim 1, wherein the compound M2 is a compound represented by a formula (221) below, 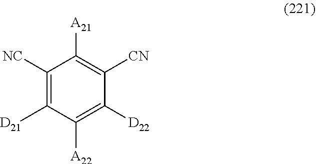
where:
A₂₁ and A₂₂ each independently represent the same as A₂; and
D₂₁ and D₂₂ each independently represent the same as D₂.

10. The organic electroluminescence device according to claim 1, wherein
Rx; R₂₀₁ to R₂₀₅ not forming the substituted or unsubstituted monocyclic ring and not forming the substituted or unsubstituted fused ring; and R₂₁₁ to R₂₁₈ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

11. The organic electroluminescence device according to claim 1, wherein
Rx; R₂₀₁ to R₂₀₅ not forming the substituted or unsubstituted monocyclic ring and not forming the substituted or unsubstituted fused ring; and R₂₁₁ to R₂₁₈ not forming the substituted or unsubstituted monocyclic ring and not forming the substituted or unsubstituted fused ring are each independently a hydrogen atom, or a substituted or unsubstituted aryl group having 6 to 50 ring carbon atoms.

12. The organic electroluminescence device according to claim 1, wherein
Rx; R₂₀₁ to R₂₀₅ not forming the substituted or unsubstituted monocyclic ring and not forming the substituted or unsubstituted fused ring; and R₂₁₁ to R₂₁₈ not forming the substituted or unsubstituted monocyclic ring and not forming the substituted or unsubstituted fused ring are each a hydrogen atom.

13. The organic electroluminescence device according to claim 1, wherein
A₂ in the compound M2 is any one group selected from the group consisting of groups represented by formulae (A21) to (A25) below, 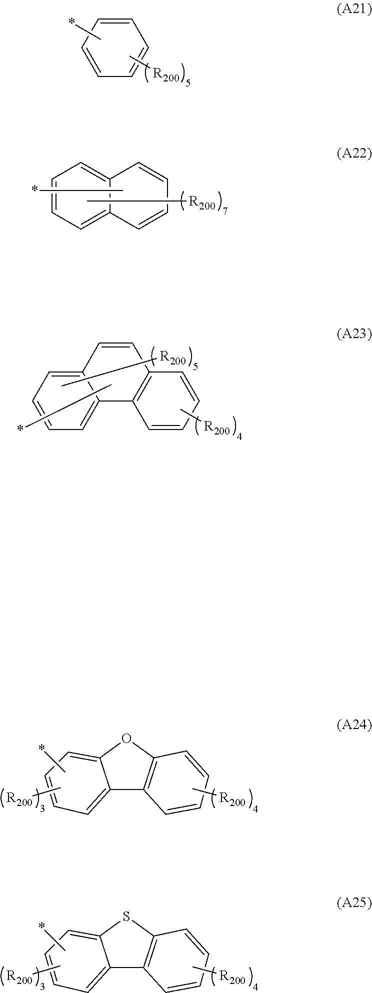
where:
at least one combination of adjacent two or more of a plurality of R₂₀₀ are mutually bonded to form a substituted or unsubstituted monocyclic ring, mutually bonded to form a substituted or unsubstituted fused ring, or not mutually bonded;
R₂₀₀ not forming the substituted or unsubstituted monocyclic ring and not forming the substituted or unsubstituted fused ring is each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms; and
* in each of the formulae (A21) to (A25) represents a bonding position to a benzene ring in the formula (2).

14. The organic electroluminescence device according to claim 13, wherein
A₂ in the compound M2 is any one group selected from the group consisting of groups represented by the formulae (A21), (A24) and (A25).

15. The organic electroluminescence device according to claim 13, wherein
A₂ in the compound M2 is a group represented by the formula (A21).

16. The organic electroluminescence device according to claim 13, wherein
A₂ in the compound M2 is a group represented by the formula (A21), and
R₂₀₀ in the formula (A21) is a hydrogen atom.

17. The organic electroluminescence device according to claim 13, wherein
R₂₀₀ in the formulae (A21) to (A25) is each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

18. The organic electroluminescence device according to claim 13, wherein
R₂₀₀ in the formulae (A21) to (A25) is each independently a hydrogen atom, or a substituted or unsubstituted aryl group having 6 to 50 ring carbon atoms.

19. The organic electroluminescence device according to claim 13, wherein
R₂₀₀ in the formulae (A21) to (A25) is a hydrogen atom.

20. The organic electroluminescence device according to claim 1, wherein
D₂ in the compound M2 is any one group selected from the group consisting of groups represented by formulae (B21) to (B23) below, 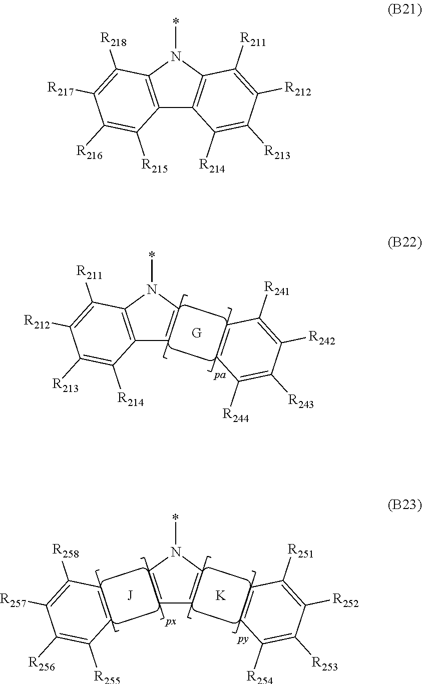
where
in the formula (B22): at least one combination of adjacent two or more of R₂₁₁ to R₂₁₄ and R₂₄₁ to R₂₄₄ are mutually bonded to form a substituted or unsubstituted monocyclic ring, mutually bonded to form a substituted or unsubstituted fused ring, or not mutually bonded;
in the formula (B23), at least one combination of adjacent two or more of R₂₅₁ to R₂₅₈ are mutually bonded to form a substituted or unsubstituted monocyclic ring, mutually bonded to form a substituted or unsubstituted fused ring, or not mutually bonded;
R₂₁₁ to R₂₁₈ in the formula (B21), R₂₁₁ to R₂₁₄ and R₂₄₁ to R₂₄₄ not forming the substituted or unsubstituted monocyclic ring and not forming the substituted or unsubstituted fused ring in the formula (B22), and R₂₅₁ to R₂₅₈ not forming the substituted or unsubstituted monocyclic ring and not forming the substituted or unsubstituted fused ring in the formula (B23) are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms;
in the formulae (B22) and (B23):
a ring G, a ring J, and a ring K are each independently any one cyclic structure selected from the group consisting of cyclic structures represented by formulae (B24) and (B25) below;
the ring G, the ring J, and the ring K are each fused with adjacent rings at any positions;
pa, px, and py are each independently 1, 2, 3, or 4;
when pa is 2, 3, or 4, a plurality of rings G are mutually the same or different;
when px is 2, 3, or 4, a plurality of rings J are mutually the same or different;
when py is 2, 3, or 4, a plurality of rings K are mutually the same or different; and
* in each of the formulae (B21) to (B23) represents a bonding position to a benzene ring in the formula (2), 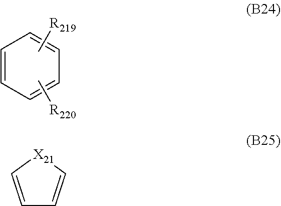
where
in the formula (B24): a combination of R₂₁₉ and R₂₂₀ are mutually bonded to form a substituted or unsubstituted monocyclic ring, mutually bonded to form a substituted or unsubstituted fused ring, or not mutually bonded;
in the formula (B25): X₂₁ is a sulfur atom, an oxygen atom, NR₂₆₁, or CR₂₆₂R₂₆₃;
a combination of R₂₆₂ and R₂₆₃ are mutually bonded to form a substituted or unsubstituted monocyclic ring, mutually bonded to form a substituted or unsubstituted fused ring, or not mutually bonded; and
R₂₆₁; R₂₁₉ and R₂₂₀ not forming the substituted or unsubstituted monocyclic ring and not forming the substituted or unsubstituted fused ring; and R₂₆₂ and R₂₆₃ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a group represented by —P(═O)(R₉₃₁)(R₉₃₂), a group represented by —Ge(R₉₃₃)(R₉₃₄)(R₉₃₅), a group represented by —B(R₉₃₆)(R₉₃₇), a substituted or unsubstituted aryl group having 6 to 50 ring carbon atoms, or a substituted or unsubstituted heterocyclic group having 5 to 50 ring atoms.

21. The organic electroluminescence device according to claim 20, wherein
R₂₁₁ to R₂₁₈ in the formula (B21), R₂₁₁ to R₂₁₄ and R₂₄₁ to R₂₄₄ in the formula (B22), R₂₅₁ to R₂₅₈ in the formula (B23), and R₂₁₉ and R₂₂₀ in the formula (B24) are each independently a hydrogen atom, a substituted or unsubstituted alkyl group having 1 to 50 carbon atoms, a substituted or unsubstituted aryl group having 6 to 50 ring carbon atoms, or a substituted or unsubstituted heterocyclic group having 5 to 50 ring atoms.

22. The organic electroluminescence device according to claim 20, wherein
R₂₁₁ to R₂₁₈ in the formula (B21), R₂₁₁ to R₂₁₄ and R₂₄₁ to R₂₄₄ in the formula (B22), R₂₅₁ to R₂₅₈ in the formula (B23), and R₂₁₉ and R₂₂₀ in the formula (B24) are each independently a hydrogen atom, or a substituted or unsubstituted aryl group having 6 to 50 ring carbon atoms.

23. The organic electroluminescence device according to claim 20, wherein
R₂₁₁ to R₂₁₈ in the formula (B21), R₂₁₁ to R₂₁₄ and R₂₄₁ to R₂₄₄ in the formula (B22), R₂₅₁ to R₂₅₈ in the formula (B23), and R₂₁₉ and R₂₂₀ in the formula (B24) are each a hydrogen atom.

24. The organic electroluminescence device according to claim 20, wherein
the formula (B22) represents any one cyclic structure selected from the group consisting of cyclic structures represented by formulae (a1) to (a6) below;
px and py in the formula (B23) are 2; and
at least one of the rings J is a cyclic structure represented by the formula (B25) and at least one of the rings K is a cyclic structure represented by the formula (B25), 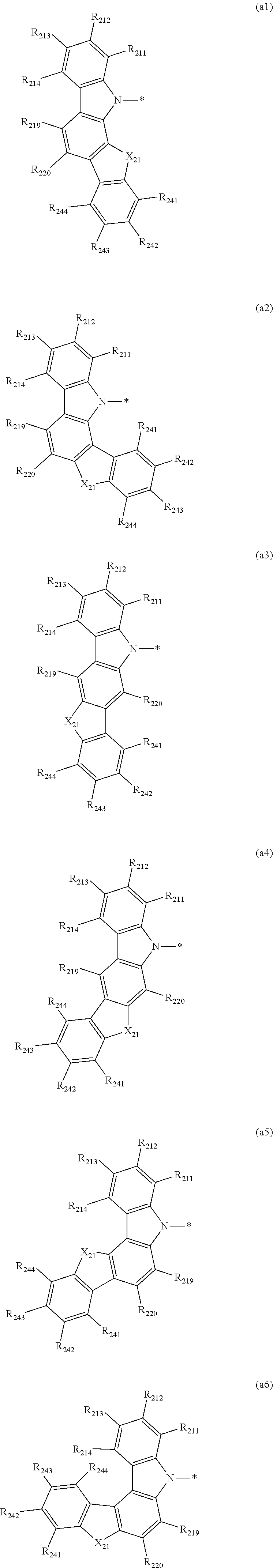
where:
R₂₁₁ to R₂₁₄ and R₂₄₁ to R₂₄₄ respectively represent the same as R₂₁₁ to R₂₁₄ and R₂₄₁ to R₂₄₄ in the formula (B22);
X₂₁, R₂₁₉ and R₂₂₀ respectively represent the same as X₂₁, R₂₁₉ and R₂₂₀ in the formula (B25); and
* in each of the formulae (a1) to (a6) represents a bonding position to a benzene ring in the formula (2).

25. The organic electroluminescence device according to claim 24, wherein
D₂ in the compound M2 is a group represented by the formula (B22) or the formula (B23).

26. The organic electroluminescence device according to claim 20, wherein
X₂₁ in the compound M2 is a sulfur atom, an oxygen atom, or CR₂₆₂R₂₆₃.

27. The organic electroluminescence device according to claim 20, wherein
X₂₁ in the compound M2 is a sulfur atom or an oxygen atom.

28. The organic electroluminescence device according to claim 20, wherein the compound M3 is a compound represented by a formula (31) or a formula (32) below, 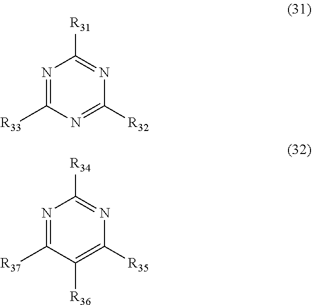
where
in the formula (32): at least one combination of adjacent two or more of R₃₅ to R₃₇ are mutually bonded to form a substituted or unsubstituted monocyclic ring, mutually bonded to form a substituted or unsubstituted fused ring, or not mutually bonded; and
R₃₁ to R₃₃ in the formula (31), R₃₄ in the formula (32), and R₃₅ to R₃₇ not forming the substituted or unsubstituted monocyclic ring and not forming the substituted or unsubstituted fused ring each independently represent the same as R₃ in the formula (3).

29. The organic electroluminescence device according to claim 28, wherein the compound M3 is a compound represented by the formula (31).

30. The organic electroluminescence device according to claim 1, wherein
L₃₁ in the compound M3 is:
a single bond;
a substituted or unsubstituted arylene group having 6 to 50 ring carbon atoms, or a trivalent, tetravalent, pentavalent, or hexavalent group derived from the arylene group; or
a divalent group formed by bonding two groups selected from the group consisting of a substituted or unsubstituted arylene group having 6 to 50 ring carbon atoms, or a trivalent, tetravalent, pentavalent, or hexavalent group derived from the divalent group; and
L₃₂ is a single bond, or a substituted or unsubstituted arylene group having 6 to 50 ring carbon atoms.

31. The organic electroluminescence device according to claim 1, wherein
L₃₁ in the compound M3 is a single bond, or a substituted or unsubstituted arylene group having 6 to 50 ring carbon atoms;
n₃ is 1; and
L₃₂ is a single bond, or a substituted or unsubstituted arylene group having 6 to 50 ring carbon atoms.

32. The organic electroluminescence device according to claim 1, wherein
L₃₁ in the compound M3 is a single bond, a substituted or unsubstituted phenylene group, a substituted or unsubstituted biphenylene group, or a divalent group formed by bonding two groups selected from the group consisting of a substituted or unsubstituted phenylene group and a substituted or unsubstituted biphenylene group, or a trivalent, tetravalent, pentavalent, or hexavalent group derived from the divalent group;
n₃ is 1; and
L₃₂ is a single bond, a substituted or unsubstituted phenylene group, or a substituted or unsubstituted biphenylene group.

33. The organic electroluminescence device according to claim 1, wherein
R₃ in the formula (3) is each independently a hydrogen atom, a substituted or unsubstituted alkyl group having 1 to 50 carbon atoms, a substituted or unsubstituted aryl group having 6 to 50 ring carbon atoms, a substituted or unsubstituted heterocyclic group having 5 to 50 ring atoms, or a group represented by the formula (3A).

34. The organic electroluminescence device according to claim 1, wherein
R₃ in the formula (3) is each independently a hydrogen atom, a substituted or unsubstituted alkyl group having 1 to 50 carbon atoms, a substituted or unsubstituted aryl group having 6 to 50 ring carbon atoms, or a group represented by the formula (3A).

35. The organic electroluminescence device according to claim 1, wherein
the compound M3 comprises, in a molecule, at least one group selected from the group consisting of groups represented by formulae (A31) to (A44) below, 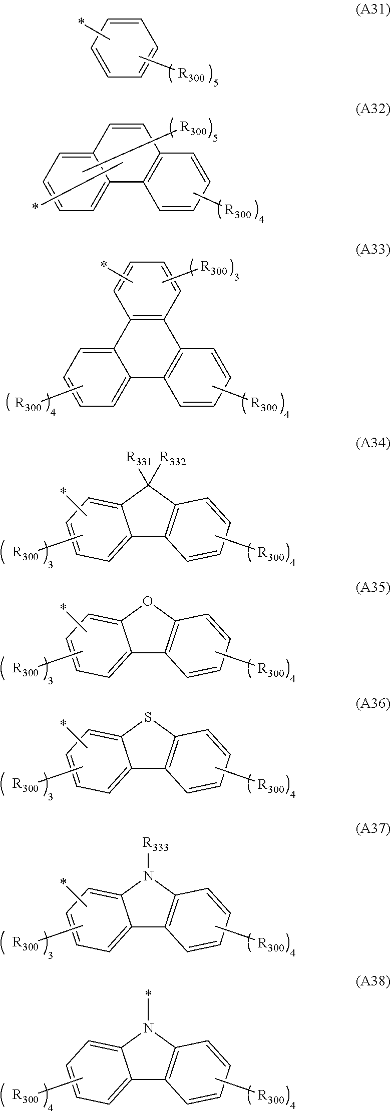
where:
at least one combination of adjacent two or more of a plurality of R₃₀₀ are mutually bonded to form a substituted or unsubstituted monocyclic ring, mutually bonded to form a substituted or unsubstituted fused ring, or not mutually bonded;
a combination of R₃₃₁ and R₃₃₂ are mutually bonded to form a substituted or unsubstituted monocyclic ring, mutually bonded to form a substituted or unsubstituted fused ring, or not mutually bonded; and
R₃₀₀, R₃₃₁ and R₃₃₂ not forming the substituted or unsubstituted monocyclic ring and not forming the substituted or unsubstituted fused ring, and R₃₃₃ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₈, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms; and
* in each of the formulae (A31) to (A38) represents a bonding position to any other atom in a molecule of the compound M3, 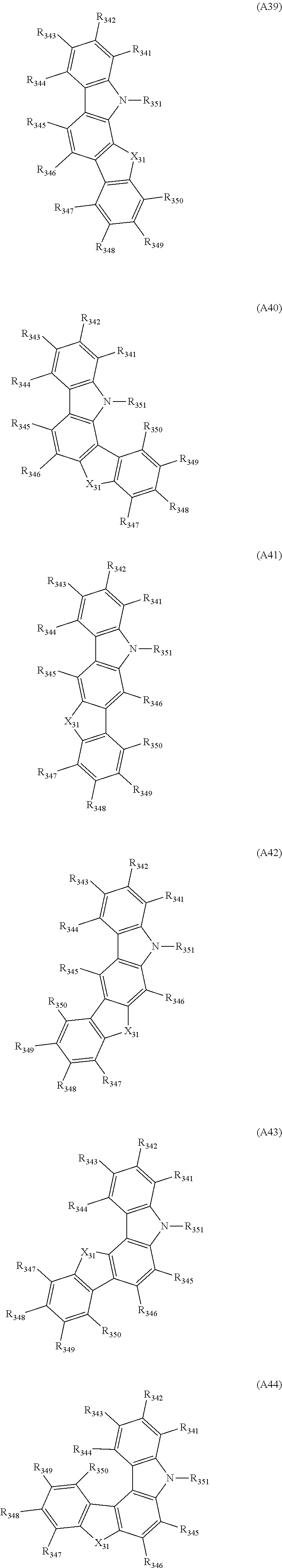
where:
at least one combination of adjacent two or more of R₃₄₁ to R₃₅₀ are mutually bonded to form a substituted or unsubstituted monocyclic ring, mutually bonded to form a substituted or unsubstituted fused ring, or not mutually bonded;
at least one of R₃₄₁ to R₃₅₁ represents a bonding position to any other atom in a molecule of the compound M3;
X₃₁ is a sulfur atom, an oxygen atom, NR₃₅₂, or CR₃₅₃R₃₅₄;
a combination of R₃₅₃ and R₃₅₄ are mutually bonded to form a substituted or unsubstituted monocyclic ring, mutually bonded to forma substituted or unsubstituted fused ring, or not mutually bonded; and
R₃₄₁ to R₃₅₁ not being a bonding position to any other atom in a molecule of the compound M3, not forming the substituted or unsubstituted monocyclic ring, and not forming the substituted or unsubstituted fused ring; R₃₅₂; and R₃₅₃ and R₃₅₄ not forming the substituted or unsubstituted monocyclic ring and not forming the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₉₀₅, a group represented by —COOR₉₀₉, a halogen atom, a cyano group, a nitro group, a substituted or unsubstituted aryl group having 6 to 50 ring carbon atoms, or a substituted or unsubstituted heterocyclic group having 5 to 50 ring atoms.

36. The organic electroluminescence device according to claim 35, wherein
the compound M3 comprises, in a molecule, at least one group selected from the group consisting of groups represented by the formulae (A38) to (A44).

37. The organic electroluminescence device according to claim 35, wherein
at least one of Y₃₁ to Y₃₆ in the compound M3 is CR₃,
at least one R₃ is a group represented by the formula (3A), and
R_{B} is any one of groups represented by the formulae (A31) to (A44).

38. The organic electroluminescence device according to claim 35, wherein
at least one of Y₃₁ to Y₃₆ in the compound M3 is CR₃, at least one R₃ is a group represented by the formula (3A), and
R_{B} is any one of groups represented by the formulae (A38) to (A44).

39. The organic electroluminescence device according to claim 1, wherein
the emitting layer further comprises a fluorescent compound M1, and
the singlet energy S₁(M2) of the compound M2 and a singlet energy S₁(M1) of the compound M1 satisfy a relationship of a numerical formula (Numerical Formula 2) below,
S₁(M2)>S₁(M1) (Numerical Formula 2).

40. The organic electroluminescence device according to claim 1, wherein
the emitting layer does not comprise a phosphorescent metal complex.

41. An electronic device comprising the organic electroluminescence device according to claim 1.
